(19) United States
(12) Patent Application Publication
(10) Pub. No.: US 2026/0306624 A1
(43) Pub. Date: Oct. 8, 2026

(54) A NOVEL NON-STOICHIOMETRIC MULTI-ELEMENT ADJUVANT (NSMA) AND COMPOSITION THEREOF

(71) Applicant: MAGVITAE INNOVATIONS INC, Gibsonia, PA (US)

(72) Inventors: Manzoor Koyakutty, Kerala (IN); Girish Madathil, Kerala (IN)

(73) Assignee: MAGVITAE INNOVATIONS INC, Gibsonia, PA (US)

(21) Appl. No.: 19/485,016

(22) PCT Filed: May 16, 2024
(86) PCT No.: PCT/IB2024/054766

§ 371 Date: Nov. 17, 2025

(30) Foreign Application Priority Data
May 16, 2023 (IN) 202321034372

(51) Int. Cl. A61K 39/39; A61K 39/00; A61K 39/215; A61K 45/06; A61P 31/14; A61P 31/20; A61P 35/00; A61P 37/04

(52) U.S. Cl. CPC A61K 39/39; A61K 39/001156; A61K 39/001192; A61K 39/215; A61K 45/06; A61P 31/14; A61P 31/20; A61P 35/00; A61P 37/04; A61K 2039/55505; A61K 2039/55555; A61K 2039/572; A61K 2039/876; C12N 2710/20034; C12N 2770/20034; C12N 2770/24134

(57) ABSTRACT

The present invention broadly relates to the field of immunology. Particularly, the present invention relates to a non-stoichiometric multi element adjuvant (NSMA) as immunomodulatory material, a method for preparing the same, a composition comprising the non-stoichiometric adjuvant. Also, the present invention relates to non-stoichiometric multi-element adjuvant (NSMA) doped with various ions and application of non-stoichiometric adjuvant in various forms of vaccine, immunotherapy and theranostics (diagnosis and therapy).

### FIELD OF THE INVENTION

The present invention broadly relates to the field of immunology. Particularly, the present invention relates to a non-stoichiometric multi-element adjuvant (NSMA) as immunomodulatory material, a method for preparing the same, a composition comprising the non-stoichiometric adjuvant. Also, the present invention relates to non-stoichiometric multi-element adjuvant (NSMA) doped with various ions and application of non-stoichiometric adjuvant in various forms of vaccine, immunotherapy and theranostics (diagnosis and therapy).

### BACKGROUND ART

The advancement of molecular genetics, protein biochemistry, peptide chemistry, and immunobiology have provided economical and efficient technologies to identify and produce large quantities of pure antigens from various pathogens. However, some of these antigens may not be sufficiently immunogenic, due to either their small size, especially synthetic peptides or the lack of intrinsic immune-stimulatory properties thereof.

Immunogenicity can be significantly improved if the antigens are co-administered with immune-enhancing materials called ‘adjuvants’. Adjuvants enhance the immunogenicity of antigens but are not necessarily immunogenic themselves. Typical adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses. In case of nano-adjuvants, the bound/conjugated antigens can be drained specifically to the nearest lymph nodes by adjusting the size and surface-chemistry of nanoparticles. Moreover, by using the selective elemental composition of nano-adjuvants, specific immunogenic pathways in antigen-presenting cells and cytotoxic T cells such as STING, NLRP3, inflammasome, TLRs, etc can be activated to boost immune response.

Immunostimulatory agents or adjuvants have been used for many years to improve the host immune responses to, for example, vaccines. Intrinsic adjuvants, such as lipopolysaccharides, normally are the components of the killed or attenuated bacteria used as vaccines. Extrinsic adjuvants are immunomodulators that are typically non-covalently linked to antigens and are formulated to enhance the host immune responses. Thus, adjuvants have been identified that enhance the immune response to antigens delivered parenterally. Some of these adjuvants are toxic, however, and can cause undesirable side-effects, making them unsuitable for use in humans and many animals. Indeed, only aluminum hydroxide and aluminum phosphate (collectively commonly referred to as alum) or calcium phosphate are used as adjuvants in human and veterinary vaccines. Recently novel adjuvants such as CpG, Mono-phosphoryl lipid A (MPL) and, QS21, a natural saponin compound extracted from the Chilean soapbark tree are approved for human applications.

It is well known that adjuvant materials are a critical part of vaccines used to enhance immunogenicity of otherwise less immunogenic antigens from infectious agents and cancer. Aluminium hydroxide or aluminium phosphate or calcium phosphates are well-known inorganic mineral adjuvants used for a variety of vaccines in the market. These materials activate some specific cellular pathways in our immune cells such as inflammasome, NLRp3, Th1 or Th2 cytokine release, etc during the vaccination and antigen presentation that help in developing antibody or cellular response against pathogens. During the last decade a number of new adjuvants are being developed by the pharma industry and put into practice in their vaccines. Lipid based adjuvants (MPLA), montanide, CpG, imiquimod, resiquimod, squalene based micro-emulation (MF series), saponin adjuvants (Iscomatrix), modified alum with lipids (AS series) are some of the new class of adjuvants recently developed or approved. However, inorganic mineral adjuvants such as Alum or calcium phosphate are most widely used as low-cost adjuvants for vaccine with better safety profile.

However, no major development happened in adjuvant science in the last 30 years because application of various elements other than Al or calcium, were least explored for immune activation. It is well documented that various inorganic elements such as zinc, magnesium, iron, cobalt, selenium, copper, etc act as essential micronutrients in our cellular functions and these elements are absorbed by the body from food materials. Although general understanding about micro-nutrients are known, it was not clear to the scientific community about the specific role and functions of each of these elements in immune system response. Further, during the infection or vaccination, these elements will not be available in appropriate concentration and essential combination in our immune cells.

Vaccine adjuvants are hotspots in vaccine research and development today. Ideal vaccine adjuvants need to be safe, effective, targeted and economical. Existing aluminium adjuvants are the longest-used and most widely used vaccine adjuvants approved for human use. It mainly relies on two mechanisms of reservoir effect and immune stimulation effect to significantly improve the body's immune response. However, as an adjuvant, it still has defects in safety and targeting. In the development of vaccines and immunogenic compositions, the common trend is the use smaller nano- or micro-sized adjuvant materials for enhancing the immune response and delivery of antigens. The recent success of nanoparticle-based COVID19 vaccines demonstrated the potential and use of many new materials such as liposomes, virus-like particles, emulsions, and oligonucleotides, as adjuvants indicating the promise of nanotechnology to enhance the efficacy of next generation vaccines.

Most of the current COVID19 vaccines as well as other viral or bacterial vaccines uses aluminium (Al) based adjuvants. Aluminium (Alum) and calcium phosphates are the only FDA approved metal ion based in organic adjuvants. Alum typically provides Th2 (Type-2) response mediated antibody production whereas calcium phosphate has better Th1 (Type 1) mediated cellular (humoral) response. From the recent COVID19 data, it is clear that an ideal vaccine adjuvant should have both antibody as well as cell-mediated immune response together with multiple other essential functionalities such as activation of toll-like receptors, STING pathway, inflammasome and cytokine/chemokine regulation. The current Alum based adjuvants can activate only inflammatory pathway and it is well established that alum cannot activate STING or TLR mechanisms of immune activation.

One of the major problems with inflammatory pathway activation by Alum is the local irritation and toxicity at the site of vaccine application. Some patients experience significant allergic response due to IgE production whereas, STING is an important aspect of antiviral immunity. Similarly, activation of interferon-gamma in T cell is a critical factor for cellular immunity and currently available alum or CaPO₄ based adjuvants has little effect on T cells. Further, none of the current metal cation-based adjuvants are known to directly activate the most important cell system that produces antibody called B cells. This demands multi-functional systems which can be engineered by combinatorial nano-engineering such that multiple functional elements can be incorporated into single amorphous nanoparticles for achieving activation of all critical immunological pathways simultaneously.

Accordingly, for the first time, the inventors of the present invention have extensively studied and developed a unique composition of nano- or micro-structure adjuvant formed by a non-stoichiometric combination of at least 2 cations and at least one anion and their application as vaccine adjuvants either alone or in combination with other.

### SUMMARY OF THE INVENTION

The present invention relates to a non-stoichiometric multi-element adjuvant (NSMA) represented by a general formula:

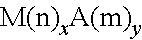

where M=at least two cations (n≥2) from the group of Zn, Al, Ca, Mn, Mo, Si, Cu, Ni, Sn, Co, Fe, Cr, Se, Na, K, and/or Mg, with weight percentage (x) varied from 0.01 to 99%,
and M is bonded with at least one (m≥1) anions, represented as: ‘A’, such as, but not limited to phosphates, poly-phosphates, phosphonates, bis-phosphonates, hydroxides, hydroxy-phosphate, hydroxy-phosphonate, oxy-hydroxy-phosphate, oxy-hydroxy phosphonate, phospho-phosphonate di or mono hydrogen-, ammonium-, sodium-, or potassium-phosphate or mono/di-hydrogen-, ammonium-sodium-, or potassium-phosphonates, sulphates, hydroxy-sulphate, hydroxy-phosphate-sulphate, hydroxy-phosphate-sulphate-phosphonate, silicates, citrates, hydroxides or combinations thereof and ‘y’ represents the weight percentage of individual anion elements. Also, the present invention provides a method for the preparation of such muti cation nano-micro adjuvant compounds and kits comprising the same.

The present invention further relates to multi-cation adjuvants doped with various other elements such as Sr, Ba, Cl, Br, I, Gd, Eu, etc for activating specific immunological pathways or properties such as fluorescence, magnetism or thermal, or surface charge.

Further, the present invention relates to a composition wherein the said non-stoichiometric multi-element adjuvant (NSMA) material is combined with bacterial, viral, fungal or cancer antigens and other excipients and wherein the composition is a vaccine composition or the adjuvant is an immunomodulator.

### BRIEF DESCRIPTION OF FIGURES

The accompanying drawings illustrate some of the embodiments of the present invention and, together with the descriptions, serve to explain the invention. These drawings have been provided by way of illustration and not by way of limitation. The components in the drawings are not necessarily drawn to scale, emphasis instead being placed upon clearly illustrating the principles of the aspects of the embodiments.

FIG. 1: A) X-ray diffraction (XRD) pattern, B) Scanning electron microscopic image (SEM) Image, C) Energy dispersive X-ray spectrum (EDAX) showing elemental composition, D) a tabular listing of element weight and the atomic ratio of a representative five cation-one anion (M(5)A(1)-1) adjuvant nanoparticle forming a calcium-rich amorphous compound: (Ca_{23.39}Mn_{9.15}Zn_{2.38}Al_{1.8}Mg_{0.54})P_{19.62}O_{43.24}

FIG. 2: A) X-ray diffraction (XRD) pattern, B) Scanning electron microscopic image (SEM) Image, C) Energy dispersive X-ray spectrum (EDAX) showing elemental composition, D) a tabular listing of element weight and atomic ratio of a representative five cation-one anion (M(5)A(1)-2) adjuvant nanoparticle forming a zinc-rich amorphous compound: (Zn_{9.72} Al_{4.7} Ca_{4.08}Mn3.97 Mg_{0.16})P1_{0.85}O_{53.79}

FIG. 3: A) X-ray diffraction (XRD) pattern, B) Scanning electron microscopic image (SEM) Image, C) Energy dispersive X-ray spectrum (EDAX) showing elemental composition, D) a tabular listing of element weight and atomic ratio of a representative four cation-one anion (M(4)A(1)-1) adjuvant nanoparticle forming a manganese-rich amorphous compound: Mn_{27.63}Zn_{5.9}Al_{3.71}Mg_{0.54})P_{18.93}O_{43.3}

FIG. 4: A) X-ray diffraction (XRD) pattern, B) Scanning electron microscopic image (SEM) Image, C) Energy dispersive X-ray spectrum (EDAX) showing elemental composition, D) a tabular listing of element weight and atomic ratio of a representative four cation-one anion (M(4)A(1)-2) adjuvant nanoparticle forming another Zinc-rich, magnesium-free amorphous compound:(Zn_{11.94}Al_{6.39}Ca_{6.39}Mn_{6.61})P_{15.9}O_{52.42}.

FIG. 5: A) X-ray diffraction (XRD) pattern, B) Scanning electron microscopic image (SEM) Image, C) Energy dispersive X-ray spectrum (EDAX) showing elemental composition, D) a tabular listing of element weight and atomic ratio of a representative four cation-one anion (M(4)A(1)-3) adjuvant nanoparticle forming another Manganese-rich amorphous compound: (Mn_{13.94}Zn_{0.86}Al_{5.98}Mg_{0.52})P_{19.77}O_{58.94}.

FIG. 6: A) X-ray diffraction (XRD) pattern, B) Scanning electron microscopic image (SEM) Image, C) Energy dispersive X-ray spectrum (EDAX) showing elemental composition, D) a tabular listing of element weight and atomic ratio of a representative four cation-one anion (M(4)A(1)-4) adjuvant microparticle forming Manganese-rich crystalline compound (Mn_{22.19}Zn_{9.03}Al_{4.99}Mg_{0.19})P_{16.65}O_{37.06}

FIG. 7: A) Scanning electron microscopic image (SEM) Image, B) Energy dispersive X-ray spectrum (EDAX) showing the elemental composition, C) tabular listing of the element weight and the atomic ratio of a representative five metal cations and three anions nanoparticle (M(5)A(3)-1) formed by (Mn_{33.9}Ca_{13.7} Zn_{5.5}Al_{4.14}Mg_{0.12})HPO4-(OH)—(PO)₂—(OH)₄—COH—(CH₂)₃NH₂. The three anions are phosphate, hydroxide and alendronate (bis-phosphonate).

FIG. 8: A) Scanning electron microscopic image (SEM) Image, B) Energy dispersive X-ray spectrum (EDAX) showing the elemental composition, C) tabular listing of the element weight and the atomic ratio of a representative five metal cations and three anions nanoparticle (M(5)A(3)-2) formed by (Mn_{6.61}Ca_{6.39}Zn1_{1.94}Al_{6.36}Mg_{0.34}) HPO4-(OH)—(PO)₂—(OH)₄—COH—(CH₂)₃NH₂

FIG. 9: A) Scanning electron microscopic image (SEM) Image, B) Energy dispersive X-ray spectrum (EDAX) showing the elemental composition, C) tabular listing of the element weight and the atomic ratio of a representative five metal cations and three anions nanoparticle (M(5)A(3)-3) formed by (Ca_{22.21}Mn_{5.7}Zn_{2.24}Al_{1.69}Mg_{0.39}) HPO4-(OH)—(PO)₂—(OH)₄—COH—(CH₂)₃NH₂ where EDAX detected P,O,C weight percentage are: P_{18.58}O_{42.04}C_{4.16}. The presence of carbon together with phosphorous and oxygen indicate presence of alendronate group.

FIG. 10: A) Scanning electron microscopic image (SEM) Image, B) Energy dispersive X-ray spectrum (EDAX) showing the elemental composition, C) tabular listing of the element weight and the atomic ratio of a representative five metal cations and three anions nanoparticle (M(5)A(3)-4) formed by HPO4-(OH)—(OP)₂—(OH)₄—(CH₂)₂ where EDAX detected P,O,C weight percentage are: P_{18.58}O_{42.04}C_{4.16}. The presence of carbon together with phosphorous and oxygen indicate presence of alendronate group.

FIG. 11: A) Scanning electron microscopic image (SEM) Image, B) Energy dispersive X-ray spectrum (EDAX) showing the elemental composition, C) tabular listing of the element weight and the atomic ratio of a representative Aluminium rich three metal cations and two anions nanoparticle (M(4)A(2)-1) formed by (Al_{10.41}Zn_{2.28}Mn_{0.85}Mg_{0.59})(OH)HP_{44.26}O_{41.93}.

FIG. 12: A) Scanning electron microscopic image (SEM) Image, B) Energy dispersive X-ray spectrum (EDAX) showing the elemental composition, C) tabular listing of the element weight and the atomic ratio of a representative Aluminium rich two metal cations and three anion nanoparticle (M(3)A(3)-1) formed by (Al_{27.07}Mn_{11.5}(OH)HP_{14.98}O_{46.03}—(OP)₂—(OH)₄—COH—(CH₂)₃NH₂).

FIG. 13. Scanning electron microscopic image (SEM) Image of a representative microcrystalline sulphate-methylene bisphosphonate based adjuvant M(4)A(2)-1: (Ca,Mg,Mn,Zn) sulphate-methylene-diphosphonate microcrystal adjuvant.

FIG. 14: A) Scanning electron microscopic image (SEM) Image, B) Energy dispersive X-ray spectrum (EDAX) showing the elemental composition, C) tabular listing of the element weight ratio of a representative Aluminium rich four metal cations and two-anion nanoparticle (M(4)A(2)-1) formed by (Al_{76.41}Zn_{17.83}Mg_{4.72}, Ca_{0.94})HPO₄—(OP)₂—(OH)₄—COH—(CH₂)₃NH₂).

FIG. 15: A) Scanning electron microscopic image (SEM) Image, B) Energy dispersive X-ray spectrum (EDAX) showing the elemental composition, C) tabular listing of the element weight ratio of a representative Aluminium rich four metal cations and two-anion nanoparticle (M(4)A(2)-1) formed by Al_{76.09}Zn_{14.56}Mg_{6.07}, Ca_{3.2})HPO₄—(OP)₂—(OH)₄—COH—(CH₂)₃NH₂).

FIG. 16: A) Scanning electron microscopic image (SEM) Image, B) Energy dispersive X-ray spectrum (EDAX) showing the elemental composition, C) tabular listing of the element weight ratio of a representative Aluminium rich four metal cations and two-anion nanoparticle (M(4)A(2)-1) formed by Al_{89.35} Zn_{0.36}Mg_{8.46}, Ca_{1.58})HPO₄—(OP)₂—(OH)₄—COH—(CH₂)₃NH₂).

FIG. 17: A) Scanning electron microscopic image (SEM) Image, B) Energy dispersive X-ray spectrum (EDAX) showing the elemental composition, C) tabular listing of the element weight ratio of a representative Aluminium rich four metal cations and two-anion nanoparticle (M(4)A(2)-1) formed by (Al_{83.9} Zn_{4.89}Mg_{7.59}Ca_{3.42})HPO₄—(OP)₂—(OH)₄—COH—(CH₂)₃NH₂).

FIG. 18: A) Scanning electron microscopic image (SEM) Image, B) Energy dispersive X-ray spectrum (EDAX) showing the elemental composition, C) tabular listing of the element weight ratio of a representative Aluminium rich four metal cations and two-anion nanoparticle (M(4)A(2)-1) formed by (Al_{72.48}Zn_{22.21} Mg_{1.1}Ca_{4.11})HPO₄—(OP)₂—(OH)₄—COH—(CH₂)₃NH₂).

FIG. 19: A) Scanning electron microscopic image (SEM) Image, B) Energy dispersive X-ray spectrum (EDAX) showing the elemental composition, C) tabular listing of the element weight ratio of a representative Aluminium rich four metal cations and two-anion nanoparticle (M(4)A(2)-1) formed by Al_{67.89}Zn_{21.07} Mg_{9.73}Ca_{1.15})HPO₄—(OP)₂—(OH)₄—COH—(CH₂)₃NH₂

FIG. 20: A) Scanning electron microscopic image (SEM) Image, B) Energy dispersive X-ray spectrum (EDAX) showing the elemental composition, C) tabular listing of the element weight ratio of a representative M(7)A(2) nanoadjuvant selenium rich, silicon, molybdenum, manganese iron, Co, Cu hydrogen phosphate-alendronate composition: Se_{40.37}Cu_{18.43} Si_{8.77} Mn_{5.54}Mo_{2.85}Fe_{8.18}Co_{10.73})HPO₄—(OP)₂—(OH)₄—COH—(CH₂)₃NH₂).

FIG. 21: A) Scanning electron microscopic image (SEM) Image, B) Energy dispersive X-ray spectrum (EDAX) showing the elemental composition, C) tabular listing of the element weight ratio of a representative M(7)A(2) nanoadjuvant selenium rich, silicon, molybdenum, manganese iron, Co, Cu hydrogen phosphate-alendronate composition: (Se_{40.37} Cu_{18.43}Si_{8.77}Mo_{2.85}Mn_{10.68}Fe_{8.18}Co_{10.73})HPO₄—(OP)₂—(OH)₄—COH—(CH₂)₃NH₂).

FIG. 22: A) Scanning electron microscopic image (SEM) Image, B) Energy dispersive X-ray spectrum (EDAX) showing the elemental composition, C) tabular listing of the element weight ratio of a representative M(7)A(2) nanoadjuvant selenium rich, silicon, molybdenum, manganese iron, Co, Cu hydrogen phosphate-alendronate composition: (Se_{63.52} Cu_{11.38} Si_{9.51} Mo_{2.57}Mn_{5.62} Fe_{3.17}Co_{4.24})HPO₄—(OP)₂—(OH)₄—COH—(CH₂)₃NH₂)

FIG. 23: Dynamic light scattering (DLS) data showing the size distribution of two types of Mₓ(n)A_{y}(m) nanoparticle adjuvants having size ranging from A) 100-200 nm range B) 800-1600 nm range.

FIG. 24: In vitro Biocompatibility of a representative adjuvant nanoparticles M(5)A(3)-1 in dendritic cells.

FIG. 25: Optical microscopic images of a vaccine for SARS-COV-2 vaccine prepared using M(5)A(3)-1 nanoparticle as adjuvant and SARS-COV2 virus RBD peptide as antigen.

FIG. 26: Optical microscopic images of a vaccine depot for cancer (melanoma) formed by using M(4)A(2)-1 Ca, Mg, Zn, Mn) SO4-(OP)₂—(OH)₄—(CH₂)₂ adjuvant and melanoma peptides (TRP2 & gp100), TLR agonists (CpG and Poly IC), Growth factor mGM-CSF).

FIG. 27: Dendritic cell activation studies showing enhanced CD86 expression, which is a bio-marker for DC activation, M(5)A(3)-1, M(5)A(3)-2, and M(5)A(3)-3 nanoparticle-adjuvant treated cells (A) Flowcytometry data B) Mean fluorescence intensity.

FIG. 28: Dendritic cell activation studies showing enhanced CD86 expression in an aluminium rich M(3)A(3)-1, nanoparticle-adjuvant compared to a clinically used Alum adjuvant used as a control.

FIG. 29: Gene expression data showing the ability of M(5)A(3) nanoparticle-adjuvants to activate various Toll-like receptors in macrophages. TLR expression is critical for innate immune activation.

FIG. 30: Assay showing ability of M(5)A(1)-1, M(5)A(1)-2, and M(5)A(1)-3 nanoparticle-adjuvants to enhance the proliferation of human mononuclear cells within 24-48 Hrs. Assay: Alamar blue.

FIG. 31: Flowcytometry assay showing the ability of inventive nanoparticle adjuvant based Human papilloma virus vaccine to enhance the B plasma cells and T follicular cell response in vaccinated mice (A) BM: Bone marrow, (B) LN: Lymph node), (Nanoparticle used: M(5)A(1)-3).

FIG. 32: ELISA data showing ability of (M(5)A(1)-3) (S+CA3 Group) nanoparticles to enhance the antibody titer when used as an adjuvant in COVID19 vaccines formed by RBD peptide. Alum based clinical vaccine was used as a positive control.

FIG. 33: ELISA data showing ability of (M(n)ₓA(m)_{y}) nanoparticle-adjuvants to enhance the virus neutralizing antibody level to >97% when vaccinated as an adjuvant in infectious disease (COVID19) vaccine. SARS-COV2 RBD peptide vaccine used as positive control.

FIG. 34: ELISPOT Data showing ability of (M(n)ₓA(m)_{y}) nanoparticle-adjuvants to enhance the interferon-gamma expressing capacity of T cells in a vaccinated animal when re-challenged with same antigen.

FIG. 35: ELISPOT Data showing ability of (M(n)ₓA(m)_{y}) nanoparticle-adjuvants to enhance the interferon-gamma expressing capacity of T cells in a commercial vaccine of Abbott.

FIG. 36: ELISA Data showing ability of (M(n)ₓA(m)_{y}) nanoparticle-adjuvants to enhance the antibody titer of commercial vaccine for Human papilloma Virus, brand-name Gardasil, manufactured by Merck.

FIG. 37: ELISPOT Data showing ability of (M(n)ₓA(m)_{y}) nanoparticle-adjuvants to enhance the interferon-gamma expressing T cell response when used in combination with commercial vaccine for Human papilloma Virus, brand-name Gardasil, manufactured by Merck.

FIG. 38: Effect Cancer-vaccination with (M(n)ₓA(m)_{y}) nanoparticle-adjuvants demonstrated in melanoma: A) Tumor volume changes with time in untreated and (M(n)ₓA(m)_{y}) nanoparticle-based vaccine treated animal (B) optical images of melanoma tumor in treated and untreated animal. Treated group 1: Nanoparticle vaccine, Treated group 2. Nanoparticle plus MDSC targeted drug 5FU.

FIG. 39: Immune cell response of M(n)A(m) nanoparticle-vaccinated animal model of melanoma indicating enhanced cellular response compared to untreated control. Nanoparticles in combination with MDSC targeted conventional chemodrug drug 5FU showed much higher response indicating the possibility of combinatorial therapeutics.

FIG. 40: Anti-tumor immune response (Tumor volume change) and effects on tumor myeloid cells of: Untreated lung tumor control, anti-TME drug 5-fluorouracil (5-FU) that eliminates MDSCs and M(n)A(m) nanoparticles combined with -TME drug 5-fluorouracil in Lewis lung carcinoma

FIG. 41: Anti-tumor lymphoid cell (Cd8 and Cd4 T cells) response in. Untreated lung tumor control, anti-TME drug 5-fluorouracil (5-FU) alone and M(n)A(m) nanoparticles in combination with anti-TME drug 5-fluorouracil in Lewis lung carcinoma

FIG. 42: Anti-tumor T cell memory and anti-tumor B cell development in. Untreated lung tumor control, anti-TME drug 5-fluorouracil (5-FU) alone and M(n)A(m) nanoparticles in combination with anti-TME drug 5-fluorouracil in Lewis lung carcinoma.

### DESCRIPTION OF THE INVENTION

The present invention is now described with reference to the tables/figures and specific embodiments, including the best mode contemplated by the inventors for carrying out the invention. This description is not meant to be construed in a limiting sense, as various alternate embodiments of the invention will become apparent to persons skilled in the art, upon reference to the description of the invention. It is therefore contemplated that such alternative embodiments form part of the present invention.

Unless defined otherwise, technical and scientific terms used herein have the same meaning as commonly understood by one of ordinary skill in the art to which this invention belongs. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Some of the terms are defined briefly here below; the definitions should not be construed in a limiting sense.

An adjuvant refers to a substance that, when added to a vaccine formulation, promotes, enhances, or prolongs the body's antigen-specific immune response. The adjuvant is also an antigen adjuvant, and the use of the vaccine can significantly improve the immune effect of the vaccine. Adjuvants have been used to enhance vaccine immunity for more than 80 years. The ability of antigens to induce protective immune responses in a host can be enhanced by combining the antigen with immunostimulants or adjuvants.

The immunoadjuvant can assist the immunogen to stimulate the body to produce an earlier, stronger and lasting immune response. The ideal immune adjuvant should meet the following conditions: no toxicity or minimal toxicity in the effective dose; it can stimulate the body to produce strong body fluids. Immune and/or cellular immune response; has long-lasting immunity; does not induce autoimmunity; no mutagenic, carcinogenic, teratogenic effects.

The term “immunostimulating agent” is typically understood not to include agents as e.g. antigens (of whatever chemical structure), which elicit an adaptive/cytotoxic immune response, e.g. a “humoral” or “cellular” immune response, in other words elicit immune responses (and confer immunity by themselves) which are characterized by a specific response to structural properties of an antigen recognized to be foreign by immune competent cells. Rather, by “immunostimulating agent”, it is typically understood to mean agents/compounds/complexes which do not trigger any adaptive/cytotoxic immune response by themselves, but which may exclusively enhance such an adaptive/cytotoxic immune response in an unspecific way, by e.g. activating “PAMP” receptors and thereby triggering the release of cytokines which support the actual adaptive/cytotoxic immune response. Accordingly, any immunostimulation by agents (e.g. antigens) which evoke an adaptive and/or cytotoxic immune response by themselves (conferring immunity by themselves directly or indirectly) is typically disclaimed by the phrase “immunostimulating agent”.

The term “adjuvant” is also understood not to comprise agents which confer immunity by themselves. Accordingly, adjuvants do not by themselves confer immunity, but assist the immune system in various ways to enhance the antigen-specific immune response by e.g. promoting presentation of an antigen to the immune system. Hereby, an adjuvant may preferably e.g. modulate the antigen-specific immune response by e.g. shifting the dominating Th1-based antigen specific response to a more Th2-based antigen specific response or vice versa. Accordingly, the terms “immunostimulating agent” and “adjuvant” in the context of the present invention are typically understood to mean agents, compounds or complexes which do not confer immunity by themselves, but exclusively support the immune response in an unspecific way (in contrast to an antigen-specific immune response) by effects, which modulate the antigen-specific (adaptive cellular and/or humoral immune response) by unspecific measures, e.g. cytokine expression/secretion, improved antigen presentation, shifting the nature of the arms of the immune response etc. Accordingly, any agents evoking by themselves immunity are typically disclaimed by the terms “adjuvant” or “immunostimulating agent”.

These adjuvants are used for activating multiple cellular and non-cellular immune mechanisms for the vaccination and immunotherapy for infectious disease, cancer and/or autoimmunity. As these various elements act us essential micro-nutrients for immune cells and their release inside the cells are a structure or crystal phase dependent phenomena, a key aspect of this invention is the selection of specific set of combination of cations and anions at appropriate ratios and their realization by precipitation chemistry.

The present invention provides a non-stoichiometric multi-element adjuvant (NSMA) comprising at least 02 elements from the group consisting of Zn, Al, Ca, Mn, Mo, Si, Sn, Ni, Cu, Cr, Co, Fe, Se, Si, Na, K and Mg, present in various weight ratios in an amorphous or polycrystalline, microcrystalline particles formed by the reaction of the combination of anions selected from at least one of phosphates, phosphonates, bis-phosphonate, hydroxy-phosphate, hydroxy-phosphonate, oxyhydroxy-phosphate, oxy-hydroxy phosphonate, phospho-phosphonate di or mono hydrogen-, ammonium-, sodium-, or potassium-phosphate or mono/di-hydrogen-, ammonium-sodium-, or potassium-phosphonates, sulfates, hydroxy-sulfate, hydroxy-phosphate-sulfate, hydroxy-phosphate-sulfate-phosphonate, silicates, citrates, and/or hydroxides.

The multi-cation adjuvants can activate different and specific immunological pathways such as, but not limited to, STING, Toll like receptors (TLR1, TLR2, TLR3, TLR4, TLR5, TLR7, TLR8, TLR9), CD86, CD80, CD40, MHC-II, MHC-I, cytokines such as interferon-alpha, interferon-gamma, interferon-beta, interleukins (ILs): IL2, IL4, IL18, IL1beta, IL12, IL6, IL8, TNF-alpha, IL10, IL5, and chemokine pathways both at genomic level and proteins level.

The multi-cations adjuvants are able to combinatorically influence, moderate, modulate, enhance, up- or down-regulate activate or inactivate specific immunological pathways such as STING, co-stimulatory pathways (CD80, CD86, CD83, CD28, CD40), MHC-I, MHC-II, Clec9, interleukins, Th1 and Th2 cytokines, chemokines, immune check point blockade proteins (PDL-1 PD-1, Lag3, TIM, IDO), inflammasome pathway, interferon regulatory factors, NF-kappa-B pathway, toll like receptors, IL18, interferons (alpha, beta, gamma), IL1-beta in all kinds of immune cells and each of the selected elements will work as co-factor for multiple biological functions in the immune system.

The non-stoichiometric multi-element adjuvant (NSMA) of the present invention is for the vaccination against infectious diseases including that caused by bacteria, virus, fungi as well as anti-cancer vaccines, and cell therapy such as dendritic cell, NK cell, T cell, CART cell therapy, anti-M2 macrophages, anti-T reg, anti-MDSC cells and for the treatment of autoimmune disorders.

### EMBODIMENTS OF THE PRESENT INVENTION

In an embodiment, the present invention provides a non-stoichiometric multi-element adjuvant (NSMA) with combination of cations and anions as represented by a general formula:

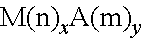

where ‘M’ represents cation elements,
‘n’ represents the number of cations used in each compound which is at least 2,
‘A’ represents the type of anion groups and ‘m’ represents the numbers of anion groups which is at least 1,
‘x’ and ‘y’ represents weight percentage of each cations or anions, respectively, varying from 0.01 to 99%.

In another embodiment, the nano-micro-adjuvant is formed by non-stoichiometric combination of at least two cations (M), selected from the group but not limited to Zn, Al, Ca, Sr, Ba, Mn, Mo, Si, Cu, Ni, Sn, Co, Fe, Cr, Se, Na, K, and/or Mg.

In another embodiment, the present invention provides the non-stoichiometric adjuvant wherein the M is formed by a combination of at least 2, 3, 4, 5, 6, 7, 8, 9, or 10 or more cations.

In another embodiment, the nano-micro-adjuvant is formed by non-stoichiometric combination of at least one anion (A), selected from the group, but not limited to PO₄, OH, HPO₄, H₂PO₄, OHPO₄, (OH)H—PO₄, (OH)H₂PO₄, [SiO₂₊ₙ]2n-, SO₄, phosphonates, bisphosphonate or combination thereof.

In a preferred embodiment, the phosphonates are represented by formula:

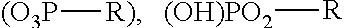

where R═CH₃, C₂H₃, C₆H₅, (CH₂)₂COOH

In a preferred embodiment, the bisphosphonates are represented by formula:

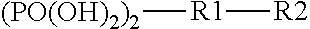

where R1=Cl, H or OH,
   R2=CH₃, Cl, (CH₂)₂NH₂, (CH₂)₅NH₂, (CH₂)₂N(CH₃)₂, (CH₂)₃NH₂, (CH₂)₂N(CH₃)(CH₂)₄(CH₃), CH₂)N(CH₂)NCH, (CH₂)N(C(CH)₄, (CH)S(C₆H₄)Cl.

In another embodiment, the bisphosphonate is formed by a) Chlodronate b) pamidronate c) alendronate c) zolendronate, d) neridronate e) etidronate f) tiludronate g) olpadronate h) Ibandronate j) risendronate, and/or h) di-methyl methyl phosphonate

In an embodiment of the present invention, the non-stoichiometric multi-element adjuvant (NSMA) having the molecular formula M(n)ₓA(m)_{y} wherein the adjuvant comprises a combination of multiple cations and at least one anion and is represented by the general formula:

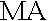

wherein M is cation and
A is anion.

In another embodiment, the present invention provides the non-stoichiometric adjuvant wherein the A is selected from (PO₄), (SO₄), (O—OH), (SO₄)(PO4), (SO₄)(OH), (SO4)(OH)(PO₄), (PO₄)(OH), (HPO₄)(OH), (H₂PO₄)(OH), (SiO₄)(OH), (OH)(PO₄)(SiO₄), PO₃—R, (PO₃—R), (PO4)(PO₃—R), (PO₂—R)(OH), (OH)₂PO)₂—R,

wherein R can be selected from ═CH₃, C₂H₃, C₆H₅, (CH₂)₂COOH.

In another embodiment, the present invention provides the non-stoichiometric adjuvant wherein the A is selected from O₂P₂—(OH)₄—C—Cl2, O₂P₂—(OH)₄—COH—CH3, O₂—P₂—(OH)₄—COH—(CH₂)₂NH₂, (M)-O₂P₂—(OH)₄—COH—(CH₂)₂N(CH₃)₂, (M)-O₂—P₂—(OH)₄—COH—(CH₂)₃NH₂, (M)-O₂P₂—(OH)₄—COH—(CH₂)₂N(CH₃)(CH₂)₄(CH₃), (M)-O₂P₂—(OH)₄—COH—(CH₂)N(CH₂)NCH, (M)-O₂P₂—(OH)₄—COH—(CH₂)N(C(CH)₄, and/or (M)-O₂P₂—(OH)₄—COH—(CH)S(C₆H₄)Cl.

In another embodiment of the present invention, the compound MA is formed by a combination of cations; M and anions; A and is selected from any of the following compounds: (M)(PO₄), (M)(SO₄), M(O—OH), (M)(SO₄)(PO4), (M)(SO₄)(OH), (M)(SO4)(OH)(PO₄), (M)(PO₄)(OH), (M)(HPO₄)(OH), (M)(H₂PO₄)(OH), (M)(SiO₄)(OH), (M)(OH)(PO₄)(SiO₄), (M)PO₃—R, (M)(PO₃—R), (M)(PO4)(PO₃—R), (M)(PO₂—R)(OH), (M)((OH)₂PO)₂—R, with R═CH₃, C₂H₃, C₆H₅, (CH₂)₂COOH, (M)-O₂P₂—(OH)₄—C—Cl2, (M)-O₂P₂—(OH)₄—COH—CH3, (M)-O₂—P₂—(OH)₄—COH—(CH₂)₂NH₂, (M)-O₂P₂—(OH)₄—COH—(CH₂)₂N(CH₃)₂, (M)-O₂—P₂—(OH)₄—COH—(CH₂)₃NH₂, (M)-O₂P₂—(OH)₄—COH—(CH₂)₂N(CH₃)(CH₂)₄(CH₃), (M)-O₂P₂—(OH)₄—COH—(CH₂)N(CH₂)NCH, (M)-O₂P₂—(OH)₄—COH—(CH₂)N(C(CH)₄, and/or (M)-O₂P₂—(OH)₄—COH—(CH)S(C₆H₄)Cl, where M is selected from at least two cations from the group of: Zn, Al, Ca, Sr, Ba, Mn, Mo, Si, Cu, Ni, Sn, Co, Fe, Cr, Se, Na, K, and/or Mg.

In an embodiment, the present invention provides the non-stoichiometric adjuvant wherein the adjuvant is a bi-cation-adjuvant represented by general formula:

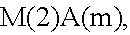

where M is formed by a combination of Zn and Al and the concentrations of Zn and Al will vary between 0.01 to 99 w %, respectively and,
A is formed by single or a combination of anions selected from the group consisting of hydroxide, sulphate, silicate, phosphate, hydrogen phosphate, bis-phosphonates, polyphosphates, oxygen, hydrogen or combinations thereof.

In an embodiment, the present invention provides the non-stoichiometric adjuvant wherein the adjuvant is a tri-cation adjuvant represented by the general formula:

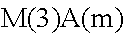

wherein M=Mn, Zn, and Al and wherein the concentration of each of these metal ions may vary from 0.01 to 99 w %, and
A is formed by anions selected from the group of hydroxides, sulphate, phosphate, silicate, hydrogen phosphate, bis-phosphonates, polyphosphates, oxygen, hydrogen, or combinations thereof.

In an embodiment, the present invention provides the non-stoichiometric adjuvant wherein the adjuvant is a tetra-cation represented by general formula:

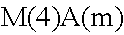

wherein M=Mn, Zn, Al, Ca and wherein the concentration of each of these metal cations may vary from 0.01 to 99 w %, and
A is formed by anions from the group of hydroxide, sulphate, phosphate, silicate, hydrogen phosphate, bis-phosphonates, polyphosphates, oxygen, hydrogen, or a combinations thereof.

In an embodiment, the present invention provides the non-stoichiometric adjuvant wherein the adjuvant is a penta-cation adjuvant represented by the general formula:

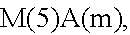

where M=Mn, Zn, Al, Ca, and Mg, and wherein the concentration of each of these metal cations may vary from 0.01 to 99 w %, and
   where A is formed by anions from the group of hydroxide, sulphate, phosphate, silicate, hydrogen phosphate, bis-phosphonates, polyphosphates, oxygen, hydrogen, or a combinations thereof.

In an embodiment, the present invention provides the non-stoichiometric adjuvant wherein the adjuvant is a hexa-cation adjuvant is represented by general formula:

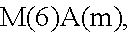

Where M=Mn, Zn, Al, Ca, Mg, and Fe and wherein the concentration of each of these metal cations may vary from 0.01 to 99 w %, and
A is formed by anions from the group of, hydroxide, sulphate, phosphate, silicate, hydrogen phosphate, bis-phosphonates, polyphosphates, oxygen, hydrogen, or combinations thereof.

In an embodiment, the present invention provides the non-stoichiometric adjuvant wherein the adjuvant is a hepta-cation adjuvant represented by general formula:

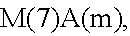

where M=Mn, Zn, Al, Ca, Mg, Fe and Se and wherein the concentration of each of these metal cations may vary from 0.01 to 99 w %, and
A is formed by anions from the group of, hydroxide, sulphate, phosphate, silicate, hydrogen phosphate, bis-phosphonates, polyphosphates, oxygen, hydrogen, or combinations thereof.

In an embodiment, the present invention provides the non-stoichiometric adjuvant wherein the adjuvant is a octa-cation adjuvant represented by general formula:

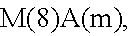

where M=Mn, Zn, Al, Ca, Mg, Fe, Se and Co and wherein the concentration of each of these metal cations may vary from 0.01 to 99 w %, and
A is formed by anions from the group of hydroxide, sulphate, phosphate, silicate, hydrogen phosphate, bis-phosphonates, polyphosphates, oxygen, hydrogen, or combinations thereof.

In an embodiment, the present invention provides the non-stoichiometric adjuvant wherein the adjuvant is a nona-cation adjuvant represented by general formula:

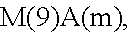

where M=Mn, Zn, Al, Ca, Mg, Fe, Se, Co and Cu and wherein the concentration of each of these metal cations may vary from 0.01 to 99 w %, and
A is formed by anions from the group of hydroxide, sulphate, phosphate, silicate, hydrogen phosphate, bis-phosphonates, polyphosphates, oxygen, hydrogen, or combinations thereof.

In an embodiment, the present invention provides the non-stoichiometric adjuvant wherein the adjuvant is a deca-cation adjuvant represented by general formula:

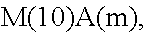

where M=Mn, Zn, Al, Ca, Mg, Fe, Se, Co, Cu and Mo and wherein the concentration of each of these metal cations may vary from 0.01 to 99 w %, and
A is formed by anions from the group of, hydroxide, sulphate, phosphate, silicate, hydrogen phosphate, bis-phosphonates, polyphosphates, oxygen, hydrogen, or combinations thereof.

In an embodiment, the present invention provides the non-stoichiometric adjuvant wherein the adjuvant is a undeca-cation adjuvant represented by general formula:

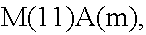

where M=Mn, Zn, Al, Ca, Mg, Fe, Se, Co, Cu, Mo, and Si and wherein the concentration of each of these metal cations may vary from 0.01 to 99 w %, and
A is formed by anions from the group of hydroxide, sulphate, phosphate, silicate, hydrogen phosphate, bis-phosphonates, polyphosphates, oxygen, hydrogen, or combinations thereof.

In another important embodiment, the present invention provides the non-stoichiometric adjuvant wherein the adjuvant comprises 5 cations are linked to at least one anion and is represented by the general formula:

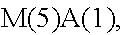

wherein M is selected from Ca,Mn,Zn,Al,Mg non-stoichiometrically linked with at least one anion A, and
wherein A is hydrogen-phosphate.

In a preferred embodiment, the 5 cation adjuvant is represented by the molecular formula:

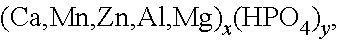

wherein weight percentage (x) of Zn can vary from 0.01-99 w %,
Al concentration can vary from 0.01-99 w %,
Ca concentration can vary from 0.01-99 w %,
Mn concentration can vary from 0.01 to 99 w %,
Mg concentration can vary from 0.01-99 w %.

In another specific embodiment, the present invention provides the non-stoichiometric adjuvant wherein the adjuvant comprises 4 cations represented by general formula:

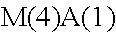

Wherein M=Al, Zn, Ca, Mg which are non-stoichiometrically linked with at least one anion; and
A is selected from hydrogen phosphate or hydrogen phosphate-bisphosphonate

In a preferred embodiment, the 4 cation adjuvant is represented by the molecular formula:

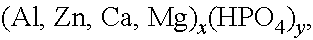

where weight percentage (x) of Al concentration range from 60 to 95 wt %
Zn concentration range from 0.1 to 25 wt %
Ca concentration range from 0.1 to 25 wt %
Mg concentration range from 0.1 to 25 wt %
And the anions are formed by either HPO4 or H2 PO4 or HPO4-alendronate.

In another embodiment of the present invention, the present invention provides the non-stoichiometric adjuvant wherein the adjuvant is an aluminium rich tetra-cation and bi-anion adjuvant represented by general formula:

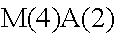

wherein M=Al, Zn, Ca, Mg and
wherein the concentration Al range from 60-95 wt %,
Ca range from 0.01-25 wt %,
Zn range from 0.01-25 wt %
Mg range 0.1-25 wt % and
A is formed by anions such as hydrogen phosphate or hydrogen phosphate in combination with bisphosphonates such as alendronate.

In a more preferred embodiment, the aluminium rich adjuvant is represented by the formula:

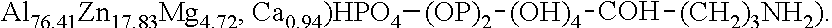

In a more preferred embodiment, the aluminium rich adjuvant is represented by the formula:

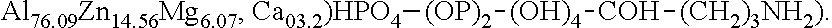

In a more preferred embodiment, the aluminium rich adjuvant is represented by the formula:

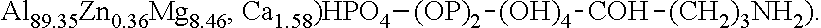

In a more preferred embodiment, the aluminium rich adjuvant is represented by the formula:

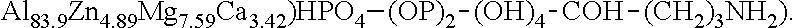

In a more preferred embodiment, the aluminium rich adjuvant is represented by the formula:

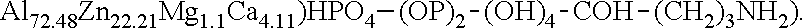

In a more preferred embodiment, the aluminium rich adjuvant is represented by the formula:

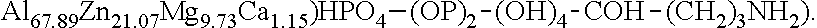

In another embodiment, the present invention provides the non-stoichiometric adjuvant wherein the adjuvant comprises 5 cations linked to at least two anions and is represented by the general formula:

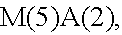

wherein M is formed of 5 cations selected from Ca,Mn,Zn,Al,Mg and two anions A selected from either hydroxyl group and/or di-hydrogen phosphate.

In a preferred embodiment, the 5 cation adjuvant is represented by the molecular formula:

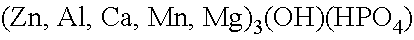

where Zn varies from 0.01-99 w %,
Al concentration varies from 0.01-99 w %,
Ca concentration varies from 0.01-99 w %,
Mn concentration vary from 0.01 to 99 w %,
Mg concentration varies from 0.01-99 w %.

In another embodiment, the present invention provides the non-stoichiometric adjuvant wherein the adjuvant comprises 5 cations linked to at least three and is represented by the general formula:

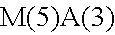

wherein M is formed of 5 cations selected from Ca,Mn,Zn,Al,Mg linked with three anions ‘A’ selected from hydroxyl group, hydrogen phosphate and a bis-phosphonate group.

In a more preferred embodiment, the 5-cation 3-anion adjuvant is represented by the molecular formula:

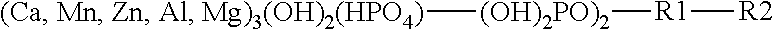

Where R1=H, OH, or Cl,
R2=CH3, Cl, (CH2)₂NH₂, (CH₂)₅NH₂, (CH₂)₂N(CH₃)₂, (CH₂)₃NH₂, (CH₂)₂N(CH₃)(CH₂)4CH₃, C7H10NNaP2O7, C5H10N2PO7, C5H12N2P2O8 and
where Zn concentration varies from 0.01-99 w %,
Al concentration varies from 0.01-99 w %,
Ca concentration varies from 0.01-99 w %,
Mn concentration vary from 0.01 to 99 w %,
Mg concentration varies from 0.01-99 w %

In another embodiment, the present invention provides the non-stoichiometric adjuvant wherein the adjuvant comprises 3 cations linked to at least two anions and is represented by the molecular formula:

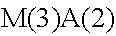

wherein M is formed of 3 cations selected from Al, Mn, Mg linked with two anions A selected from hydroxyl group and/or hydrogen-phosphate or bisphosphonate group.

In a more preferred embodiment, the non-stoichiometric adjuvant is represented by the molecular formula: (Al—Mn—Mg)₃(OH)₂(H₂PO₄)
where Al concentration varies from 0.01-99 w %,
Mn concentration vary from 0.01 to 99 w %,
Mg concentration varies from 0.01-99 w %,

In another embodiment, the present invention provides the non-stoichiometric adjuvant wherein the adjuvant comprises 2 cations linked with two anions and is represented by the general formula:

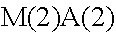

Wherein M is formed of 2 cations selected from Al, Mn linked with two anions A selected from hydroxyl group and hydrogen-phosphate.

In a preferred embodiment, the non-stoichiometric adjuvant is represented by the molecular formula:

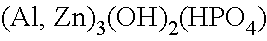

where Al concentration varies from 0.01-99 w %,
Zn concentration vary from 0.01 to 99 w %.

In another embodiment, the present invention provides the non-stoichiometric adjuvant wherein the adjuvant comprises 2 cations linked with three anions and is represented by the general formula:

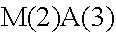

wherein M is formed of 3 cations selected from Al, Zn, and Mg linked with three anions A selected from hydroxyl group, di-hydrogen-phosphate group and bis-phosphonate groups.

In a preferred embodiment, the non-stoichiometric adjuvant is represented by the molecular formula:

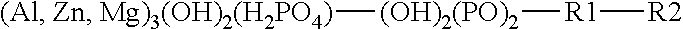

where R1=H, OH, or Cl and
R2=CH3, Cl, (CH2)₂NH2, (CH₂)₅NH₂, (CH₂)₂N(CH₃)₂, (CH₂)₃NH₂, (CH₂)₂N(CH₃)(CH₂)4CH₃, C₇H10NNaP2O7, C5H10N2PO7, C5H12N2P2O8
where Al concentration varies from 0.01-99 w %,
Zn concentration varies from 0.01 to 99 w %,
Mg concentration varies from 0.01-99 w %.

In another embodiment, the present invention provides the non-stoichiometric adjuvant comprises of 3 cations linked with three anions and is represented by the general formula:

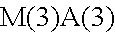

wherein M is formed of 3 cations selected from Al, Zn, Mg linked with three anions A selected from hydroxyl group, di-hydrogen-phosphate group and a bis-phosphonate group

In a preferred embodiment, the non-stoichiometric adjuvant is represented by the molecular formula:

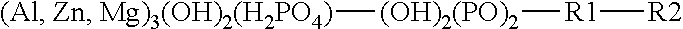

where R1=H, OH, or Cl, and
R2=CH3, Cl, (CH2)₂NH2, (CH₂)₅NH₂, (CH₂)₂N(CH₃)₂, (CH₂)₃NH₂, (CH₂)₂N(CH₃)(CH₂)4CH₃, C7H10NNaP2O7, C5H10N2PO7, C5H12N2P2O8,
where Al concentration varies from 0.01-99 w %,
Zn concentration vary from 0.01 to 99 w %,
Mg concentration varies from 0.01-99 w %.

In another embodiment, the present invention provides the non-stoichiometric adjuvant is a selenium rich adjuvant comprising 7 cation linked with 2 anions and represented by the general formula:

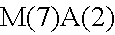

In a preferred embodiment, the non-stoichiometric adjuvant is represented by the molecular formula:

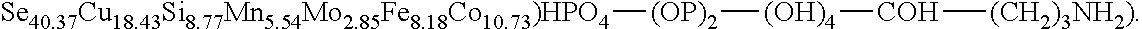

In a preferred embodiment, the non-stoichiometric adjuvant is represented by the molecular formula:

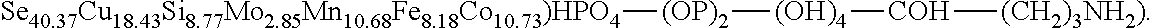

In a preferred embodiment, the non-stoichiometric adjuvant is represented by the molecular formula:

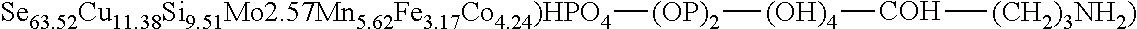

In another embodiment, the present invention provides a non-stoichiometric multi-element adjuvant (NSMA) as shown in Formula I:

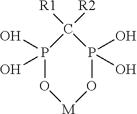

wherein (M) is at least two or more metal ions linked with bisphosphonates having the formula: M-(PO(OH)₂)₂—R1-R2,
R1=H, OH or CH₃,
R2=CH₃, Cl, (CH₂)₂NH₂, (CH₂)₅NH₂, (CH₂)₂N(CH₃)₂, (CH₂)₃NH₂, (CH₂)₂N(CH₃)(CH₂)₄(CH₃), CH₂)N(CH₂)NCH, (CH₂)N(C(CH)₄, (CH)S(C₆H₄)Cl, and
wherein M is a combination of at least two or more cations selected from the group consisting of Mn, Zn, Ca, Ba, Sr, Al, Mg, Se, Cu, Fe, Mo, Cr, Si, Ba, Sr, Sn, K, Na or combinations thereof.

In another embodiment of the present invention is to provide the non-stoichiometric adjuvant is optionally doped with other elements for creating specific properties such as florescence, magnetism, thermal response and/or zeta potential modulation.

In another embodiment, the present invention provides other non-stoichiometric adjuvants that can be selected from the group consisting of (Zn,Al,Ca,Mn,Mg)ₓ(PO₄)_{y}, (Zn,Al,Ca,Mn,Mg)ₓ (HPO₄)_{y}, (Zn,Al,Ca,Mn,Mg)ₓ (H₂PO₄)_{y}, (Zn,Al,Ca,Mn,Mg)ₓ(PO₄)_{y}(OH)_{z}, (Zn,Al,Ca,Mn,Mg)ₓ(HPO₄)_{y}(OH)_{z}, (Zn,Al,Ca,Mg)ₓ(H₂PO₄)_{y}(OH)_{z}, and wherein the weight ratios x, y and z varies from 0.01 to 99%.

In another embodiment, the present invention provides the non-stochiometric adjuvants adjuvant compounds comprising the molecular formula represented by:

| | |
|---|---|
| (Zn, Al, Ca, Mn, Mg)(PO4)₂ | (Zn, Al, Ca, Mn, Mg)₂(PO₄)₃(OH)₃ |
| (Zn, Al, Ca, Mn, Mg)(HPO4)₂ | (Zn, Al, Ca, Mn, Mg)₂(HPO₄)₃(OH)₃ |
| (Zn, Al, Ca, Mn, Mg)(H₂PO4)₂ | (Zn, Al, Ca, Mn, Mg)₂(H₂PO₄)₃(OH)₃ |
| (Zn, Al, Ca, Mn, Mg)(HPO4) | (Zn, Al, Ca, Mn, Mg)₃(PO4)₂ |
| (Zn, Al, Ca, Mn, Mg)((HPO₄)(H₂O)₂ | (Zn, Al, Ca, Mn, Mg)₃(HPO4)₂ |
| (Zn, Al, Ca, Mn, Mg)(PO₄)₂(OH)₃ | (Zn, Al, Ca, Mn, Mg)₃(H₂PO4)₂ |
| (Zn, Al, Ca, Mg)(HPO₄)₂(OH)₃ | (Zn, Al, Ca, Mn, Mg)₃(PO4)₂(OH)₃ |
| (Zn, Al, Ca, Mn, Mg)(H₂PO₄)₂(OH)₃ | (Zn, Al, Ca, Mn, Mg)₃(HPO4)₂(OH)₃ |
| (Zn, Al, Ca, Mg)₂(PO4)₃ | (Zn, Al, Ca, Mn, Mg)₃(H₂PO4)₂(OH)₃ |
| (Zn, Al, Ca, Mn, Mg)₂(HPO4)₃ | (Zn, Al, Ca, Mn, Mg)₃(PO₄)(OH)₂ |
| (Zn, Al, Ca, Mn, Mg)₂(H₂PO4)₃ | (Zn, Al, Ca, Mn, Mg)₃(HPO₄)(OH)₂ |
| (Zn, Al, Ca, Mn, Mg)₃(H₂PO₄)(OH)₂ | (Zn, Al, Ca, Mn, Mg)₄(PO₄)₃ |
| (Zn, Al, Ca, Mn, Mg)₄(HPO₄)₃ | (Zn, Al, Ca, Mn, Mg)₄(H₂PO₄)₃ |
| (Zn, Al, Ca, Mn, Mg)₄(PO₄)₃(OH)₄ | (Zn, Al, Ca, Mn, Mg)₄(HPO₄)₃(OH)₄ |
| (Zn, Al, Ca, Mn, Mg)₄(H₂PO₄)₃(OH)₄ | (Zn, Al, Ca, Mn, Mg)₆(PO₄)₄(OH)₈ |
| (Zn, Al, Ca, Mn, Mg)₆(HPO₄)₄(OH)₈ | (Zn, Al, Ca, Mn, Mg)₆(H₂PO₄)₄(OH)₈ |
| (Zn, Al, Ca, Mg)₈H₂(PO₄)₆ | (Zn, Al, Ca, Mn, Mg)(PO₄)₆PO₃OH |
| (Zn, Al, Ca, Mn, Mg)₂P₂O₇ | (Zn, Al, Ca, Mn, Mg)₅(P₃O₁₀)₂ |
| (Zn, Al, Ca, Mn, Mg)₅(PO₄)₃(OH) | (Zn, Al, Ca, Mg)₁₀(PO₄)₆(OH, F, Cl, Br, I)₂ |
| (Zn, Al, Ca, Mn, Mg)₄(PO₄)₂O | (Zn, Al, Ca, Mg)₃(H₂PO₄)(OH)₂—C—R1—R2 |
| | where R1 = H, OH, Cl and R2 = CH₃, Cl, |
| | (CH2)₂NH2, (CH₂)₅NH₂, (CH₂)₂N(CH₃)₂, |
| | (CH₂)₃NH₂, (CH₂)₂N(CH₃)(CH₂)4CH₃, |
| | C₇H₁₀NNaP2O₇, C₅H₁₀N₂PO₇, or |
| | C₅H₁₂N₂P2O₈ |
| (Mn, Al, Ca, Zn, Mg)((OH)₂PO)₂—C—H—R2 | (Al, Ca, Zn, Mg)((OH)₂PO)₂—C—OH—R2 |
| R2 = CH3, Cl, (CH2)₂NH2, (CH₂)₅NH₂, | R2 = CH₃, Cl, (CH2)₂NH2, (CH₂)₅NH₂, |
| (CH₂)₂N(CH₃)₂, (CH₂)₃NH₂, | (CH₂)₂N(CH₃)₂, (CH₂)₃NH₂, |
| (CH₂)₂N(CH₃)(CH₂)4CH₃, C7H10NNaP2O7, | (CH₂)₂N(CH₃)(CH₂)4CH₃, C7H10NNaP2O7, |
| C5H10N2PO7, or C5H12N2P2O8 | C5H10N2PO7, or C5H12N2P2O8 |
| (Al, Ca, Zn, Mg)((OH)₂PO)₂—C—Cl2—R2 where | (Al, Ca, Zn, Mg)H(PO)₄—C—H—R2 |
| R2 = CH3, Cl, (CH2)₂NH2, (CH₂)₅NH₂, | R2 = CH3, Cl, (CH2)₂NH2, (CH₂)₅NH₂, |
| (CH₂)₂N(CH₃)₂, (CH₂)₃NH₂, | (CH₂)₂N(CH₃)₂, (CH₂)₃NH₂, |
| (CH₂)₂N(CH₃)(CH₂)4CH₃, C7H10NNaP2O7, | (CH₂)₂N(CH₃)(CH₂)4CH₃, C7H10NNaP2O7, |
| C5H10N2PO7, or C5H12N2P2O8 | C5H10N2PO7, or C5H12N2P2O8 |
| (Al, Ca, Zn, Mg)(H)₂PO)₄ | (Al, Ca, Zn, Mg)(H)₂PO)₄—C—H—R2 |
| | R2 = CH3, Cl, (CH2)₂NH2, (CH₂)₅NH₂, |
| | (CH₂)₂N(CH₃)₂, (CH₂)₃NH₂, |
| | (CH₂)₂N(CH₃)(CH₂)4CH₃, C7H10NNaP2O7, |
| | C5H10N2PO7, or C5H12N2P2O8 |
| Al_{76.41}Zn_{17.83}Mg_{4.72}, Ca_{0.94})HPO₄—(OP)₂— | Al_{76.09}Zn_{14.56}Mg_{6.07}, Ca_{3.2})HPO₄—(OP)₂— |
| (OH)₄—COH—(CH₂)₃NH₂). | (OH)₄—COH—(CH₂)₃NH₂). |
| Al_{89.35}Zn_{0.36}Mg_{8.46}, Ca_{1.58})HPO₄—(OP)₂— | (Al_{83.9}Zn_{4.89}Mg_{7.59}Ca_{3.42})HPO₄—(OP)₂—(OH)₄— |
| (OH)₄—COH—(CH₂)₃NH₂). | COH—(CH₂)₃NH₂). |
| (Al_{72.48}Zn_{22.21}Mg_{1.1}Ca_{4.11})HPO₄—(OP)₂— | Al_{67.89}Zn_{21.07}Mg_{9.73}Ca_{1.15})HPO₄—(OP)₂— |
| (OH)₄—COH—(CH₂)₃NH₂). | (OH)₄—COH—(CH₂)₃NH₂ |
| Se_{40.37}Cu_{18.43}Si_{8.77}Mn_{5.54}Mo_{2.85} | Se_{40.37}Cu_{18.43}Si_{8.77}Mo_{2.85} |
| Fe_{8.18}Co_{10.73})HPO₄—(OP)₂—(OH)₄—COH— | Mn_{10.68}Fe_{8.18}Co_{10.73})HPO₄—(OP)₂—(OH)₄— |
| (CH₂)₃NH₂). | COH—(CH₂)₃NH₂). |
| Se_{63.52}Cu_{11.38}Si_{9.51}Mo2.57Mn_{5.62} | |
| Fe_{3.17}Co_{4.24})HPO₄—(OP)₂—(OH)₄—COH— | |
| (CH₂)₃NH₂) | |

In another embodiment, the present invention provides a method for preparing the non-stoichiometric nano-micro particle adjuvant using wet chemical methods.

In another embodiment, the present invention provides an amorphous composition comprising the multi-element non-stoichiometric nano-micro particle adjuvant having a concentration in the range from 0.1-500 mg/g of the total compound.

In another embodiment, the present invention provides that the non-stoichiometric nano-micro sized adjuvant compound in the form of spherical particles, needles, rods, flower like structures, petals, star-like structures, prismatic structures, porous structures, and irregular self-assembled composite or core-shell structures with other materials such as proteins, peptides, RNA, DNA, carbohydrates, polymers, lipids, liposomes, lipo-proteins, sugars etc. for the purpose of immuno-activation, vaccination, and disease therapeutics and medical imaging.

In another embodiment, the present invention provides the non-stoichiometric adjuvant wherein the size of the adjuvant compound ranges from 1 nm to 10,000 μm, specifically 1-100 nm, 100-500 nm, 500-1000 nm, 1-10 μm, 10-50 μm, 50-100 μm, 100-500 μm, 500-1000 μm, 1000-2000 μm, 2000-3000 μm, 3000-4000 μm, 4000-5000 μm, 5000-6000 μm, 6000-7000 μm, 7000-8000 μm, 8000-9000 μm, 9000-10000 μm.

In another embodiment, the present invention provides the non-stoichiometric adjuvant wherein the adjuvant is further doped with metallic and non-metallic impurities such as Fe, Mo, Cu, Na, K, Co, Si, Sn, Se, F, Cl, Br, I, Gd, Y, Tc, etc. to enhance its adjuvant activity and/or image contrast properties.

In another embodiment, the present invention provides that the non-stoichiometric adjuvant enhances the efficacy of therapeutic and prophylactic vaccines, enhancing the immunogenicity of antigens such as peptides, RNA, mRNA, DNA, whole virus, bacteria, fungi, and better production of antibodies, cytokines and chemokines and to the methods and compositions for preparing and using them for medicinal applications.

In another embodiment, the present invention provides the non-stoichiometric adjuvant wherein the adjuvant is an injectable or implantable micro- or nanostructures for vaccination and immunotherapy against cancer and infectious diseases.

In another embodiment, the present invention provides a composition comprising the nano- or micro-structures of the non-stoichiometric adjuvant as claimed in claim 1, and the pharmaceutically acceptable excipients and the composition optionally comprises one or more active components are selected from:
a. immunogenic antigen;
b. antibiotics; or
c. immune-stimulating materials.
wherein the composition is a vaccine composition or an immunomodulator composition.

In another embodiment, the present invention provides that the immunogenic antigen is selected from proteins, peptides, carbohydrates, nucleic acids such as RNA, mRNA, dsRNA, miRNA, DNA, oligonucleotides, RNA/DNA-mimics, lipids, lipo-proteins, inactivated whole cancer cells, virus, bacteria, fungi, related to any type of cancer, infectious agents such as bacteria, virus, fungi or parasites.

In another embodiment, the present invention provides that antibiotics are selected from neomycin, polymyxin, Gentamicin, and/or formaldehyde.

In another embodiment, the present invention provides that the immune-stimulating materials is selected from amorphous aluminum hydroxyphosphate sulfate (AAHS), aluminum hydroxide, aluminum phosphate, potassium aluminum sulfate (Alum), Monophosphoryl lipid A (MPL) and QS-21, a natural compound extracted from the Chilean soapbark tree, combined in a liposomal formulation, Cytosine phosphoguanine (CpG), a synthetic form of DNA that mimics bacterial and viral genetic material, Saponins derived from the soapbark tree (Quillaja saponaria Molina), Oil in water emulsion composed of squalene, poly-IC, PLY-IC-LC, or combinations thereof.

In another embodiment, the present invention provides that the excipients are selected from surfactants, stabilizers, or polymers.

In another embodiment, the present invention provides that the stabilizers are selected from Monosodium glutamate (MSG) and 2-phenoxyethanol, lactose, sucrose, glycine, serum albumin, Gelatin, L-histidine, polysorbate 80, sodium borate, yeast protein, sodium chloride, monobasic sodium phosphate, dibasic sodium phosphate, monobasic potassium phosphate, potassium chloride, calcium chloride, sodium taurodeoxycholate, ovalbumin, sucrose, neomycin sulphate, polymyxin B, betapropiolactone, hydrocortisone, thimerosal, octoxynol-10 (TRITON X-100), α-tocopheryl hydrogen succinate, ovalbumin, sodium deoxycholate, sodium phosphate-buffered isotonic sodium chloride, potassium dihydrogen phosphate, cholesterol, sodium dihydrogen phosphate dihydrate, disodium phosphate anhydrous, dipotassium phosphate, and/or phenoxyethanol.

In another embodiment, the present invention provides that the immunogenic antigens is selected from other pathogens such as bacteria and fungi causing diseases selected from the group consisting of Tuberculosis, Anthrax, Tetanus, Leptospirosis, Pneumonia, Cholera, Botulism Pseudomonas Infection, MRSA Infection, E. Coli Infection, Meningitis, Gonorrhea, Bubonic Plague, Syphilis.

In another embodiment, the present invention provides that composition further comprises other adjuvants selected from Granulocyte-monocyte colony stimulating factor (GM-CSF), granulocyte-colony stimulating factor (G-CSF), monocyte-colony stimulating factor (m-CSF), saponins, imiquimod, Resiquimod (R-848), interleukins IL-1 to IL30; more specifically IL2, IL4, IL-5, IL12, IL13, IL20, IL15, IL21, IL1beta, interferon-alpha, -beta, -gamma, phosphonates, bisphosphonates, polyinosinic:polycytidylic acid, cell growth factors, transcription factors, chemokines, polysaccharides, hormones, immunogenic lipids/polymers/proteins, peptides, nucleic acids, Freund's Complete or incomplete adjuvant, lipo-polysacharide, lecithin, Lyso-lecithin, lipids, cholesterol, surfactants, tween20, tween 80, tween 100, span, Aluminum-based mineral salts (Alum), Calcium phosphate, AS04—contains 3-O-desacyl-4′-monophosphoryl lipid A (MPL), emulsions (e.g., MF59, Montanides), AS01B, CpG motifs (for eg; CpG 1018), Poly IC, AS03, ISCOMS, cholera toxin (CT), heat labile toxin (LT) from E. coli, virus-like particles, polymer adjuvant particles (e.g., PLG), virosomes, liposomal adjuvants, solid lipid adjuvants, carbohydrate adjuvants, synthetic DNA, viral like particles, RNA mimics, water in oil and oil in water containing olive, palm, coconut, peanut oil, squalene, mineral oils, aluminum monostearate; mineral gels such as aluminum hydroxide, alum, amorphous aluminium phosphate sulfate, aluminium adjuvants, calcium phosphate, aluminum phosphate hexadecylamine, octadecylamine, dimethyldioctadecyl-ammonium bromide, N,N-dioctadecyl-N′,N′-bis(2-hydroxymethyl) propanediamine, methoxyhexadecylglycerol and pluronic polyols; polyanions such as pyran, protamine sulfate, polyethyleneimne, dextran sulfate, polyacrylic acid and carbopol or combinations thereof.

In another embodiment, the present invention provides use of the non-stoichiometric adjuvant in combination with other therapy of cancer and infectious or autoimmune diseases such as chemotherapeutics, small molecule inhibitors, immunotherapy, antibody therapy, cytokine therapy, radiation therapy, nuclear medicine, photodynamic therapy, tumour killing field therapy, surgery, anti-viral therapy, ayurvedic, homeopathic and/or traditional medicine therapies.

In another embodiment, the present invention provides that use of the composition comprising the non-stoichiometric adjuvant compound for the treatment of cancer in a subject or treatment of infectious disease or treatment of immune disorders including autoimmunity

In another embodiment, the present invention provides that use of the non-stoichiometric adjuvant compound alone or in combination with one or more cell therapies or antibody therapies wherein the cell therapy is selected from CART-T cell, DC cell vaccine, NK cell therapy, adoptive T cell therapies, and/or stem-cell therapy and wherein the antibody therapy is selected from immune-checkpoint blockade antibodies against PD1, PDL1, CTLA4, Lag3, IDO, TIM3, growth factor binding antibodies against EGFR, PDGFR, VEGF, VEGFR, Her2/nue, IL6, TNF alpha inhibitor antibody.

In another embodiment, the present invention provides a method of treating a subject by generating an immune response in subject injected with the composition, wherein the immune response is generated by way of activating innate and adaptive immune cells such as DC, macrophages, B cells and T cells and producing specific and non-specific antibodies and T, NK cell, Dendritic cell, macrophage, neutrophil response against the disease condition or antigen used in the vaccine formulation.

In another embodiment, the present invention provides that a method of treating a subject by generating anti-tumor immune response against cancer, wherein the method comprises administering the composition, comprising the said adjuvant as defined in claim 1 conjugated with tumor antigens, tumor associated antigens, or neo-antigens in the form of sub-unit peptides, mRNA, RNA, DNA, polypeptide, full length proteins, carbohydrates, glycoproteins, membrane proteins, inactivated whole tumor cells, whole tumor lipid lysate, whole tumor protein lysate, whole tumor lysate, other clinically relevant adjuvants or combinations thereof.

In another embodiment, the present invention provides that the tumor specific antigens is selected from Tyrosinase-related protein (TRP)-2, Melanoma-associated Antigen-1 (MAGE-1), interleukin-13 (IL-13) receptor a2, Glycoprotein 100 (gp10O), Antigen isolated from Immunoselected Melanoma-2 (AIM-2), Chondroitin sulfate proteoglycan 4 (CSPG4), High-molecular-weight melanoma-associated antigen (HMW-MAA), Melanoma proteoglycan (MPG), Melanoma antigen recognized by T-Cell-1 (MART-1), B melanoma antigen 1 (BAGE), Integrin α3β1, Transmembrane protein-tyrosine-phosphatases (PTP-LAR), CUB domain-containing protein 1 (CDCP1), The transferrin receptor 1 (TfR1), Prostatic acid phosphatase (PAP), Alpha-fetoprotein (AFP), Carcinoembryonic antigen (CEA), Mucin-1 (MUC-1), Epithelial tumor antigen (ETA), Melanoma associated antigen. (MAGE, Tumor protein p53, Annexin A2, Tropomodulin 3 (TMOD3), disintegrin and a metalloprotease 9 (ADAM9), Glypican-3 (GPC3), Hepatocellular carcinoma-associated antigen-587 (HCA587), Cancer/testis antigen 1B (CTAG1B), Human epidermal growth factor receptor 2 (HER-2/neu), Oncoprotein E6 and E7 of human papillomavirus 16 (HPV-16), Cellular retinoic acid-binding protein 1 (CRABP1), Folate receptor alpha (FOLR1), Kallikrein Related Peptidase (KLK1O), Kallikrein-related peptidase 2 (KLK2/hK2), Human telomerase reverse transcriptase (hTERT), Cytotoxic T lymphocyte antigen 4 (CTLA-4), Prostate specific antigen (PSA), Prostate-Specific Membrane Antigen (PSMA), Hepatitis B surface antigen, Telomerase reverse transcriptase (TERT), Survivin, Lectin, galactoside binding Galectin-3 (LGALS3)Lectin, galactoside binding, soluble, Galectin-8 (LGALS8), v-Ki-ras2 Kirsten rat sarcoma virus oncogene homolog (KRAS), Embryonic stem cell expressed Ras (ERAS), Calcium voltage-gated channel auxiliary subunit gamma 1 (CACNG1), Alanyl aminopeptidase (ANPEP), F-Box Protein 6 (FBXO6), Endothelin Converting Enzyme 1 (ECE1), Tetraspanin-13 (TSPAN13), Neuron Cell Adhesion Molecule (N-CAM), Carcinoembryonic antigen (CEA), Epithelial cell adhesion molecule (Ep-CAM), Sialyl Tn sugar chains, Lewis antigens (Lewis-x, Lewis-b, Lewis-y structure), Globo H sugar chains, gangliosides such as GD2/GD3/GM2, Carbohydrate antigen 125, Carbohydrate antigen 199 Carbohydrate antigen 15-3, Tumor-associated glycoprotein 72 (TAG-72), Valosin-containing protein (p97), CD20, CD21, Mucin-16 (MUC-16), Epidermal growth factor receptor (EGFR), C-Met, receptor tyrosine kinase belonging to MET (MNNG HOS transforming gene) family, Tumor necrosis factor-related apoptosis-inducing ligand (TRAIL-R1), Insulin-Like Growth Factor 1 Receptor (IGF-1R), Vascular endothelial growth factor receptor (VEGF-R2/R1), Prostate stem cell antigen (PSCA), Growth/differentiation factor 8 (GDF8), Teratocarcinoma-derived growth factor 1 (TDGF1), Mucin-5AC gel-forming glycoprotein (MUCSAC), Carcinoembryonic antigen related cell adhesion molecules (CEACAM), Choline transporter-like protein 4 (SLC44A4), Neuropilin1, Glypican-3 (GPC3), EPH receptor A2 (EphA2), C-lectin, Pec60, Carbonic anhydrase IX (CAIX, MN, G250), Fibroblast activation protein α (FAPα, seprase), Matrix metalloproteinases (MMPs), Tumor endothelial markers (TEMs), among them TEM1 and TEM8, and/or TGF BETA receptor (CD105).

In another embodiment, the present invention provides that the cancer is lung, kidney, breast, pancreas, brain, colon, intestine, skin, liver, neuroendocrine organs, oesophagus, stomach, blood cells, muscle cells, bone, head & neck, adenocarcinoma, squamous cell carcinoma, stromal cell carcinoma, soft-tissue carcinoma, sarcomas, lymphoma, myeloma, by way of nanoparticle alone by activating immune cells, or in combination with other immunotherapeutics such as anti-MDSC therapeutics, 5fluorourasil, capecitabin, Gemcitabine, or combinations thereof.

In another embodiment, the present invention provides a vaccine composition wherein the nano- or micro-structures of the non-stoichiometric adjuvant are loaded/complexed/conjugated with an immunogenic antigen.

In another embodiment, the present invention provides an immunogenic pharmaceutical vaccine composition wherein the nano- or micro-structures of the non-stoichiometric adjuvant are mixed with single or combination of other adjuvants, and immune-stimulating materials selected from amorphous aluminum hydroxyphosphate sulfate (AAHS), aluminum hydroxide, aluminum phosphate, potassium aluminum sulfate (Alum), Monophosphoryl lipid A (MPL) and QS-21, a natural compound extracted from the Chilean soapbark tree, combined in a liposomal formulation, Cytosine phosphoguanine (CpG), a synthetic form of DNA that mimics bacterial and viral genetic material, Saponins derived from the soapbark tree (Quillaja saponaria Molina), Oil in water emulsion composed of squalene, poly-IC, PLY-IC-LC, or combinations thereof.

In another embodiment, the present invention provides the composition comprising the non-stoichiometric adjuvant particle as and when used to make a vaccine against viruses such as adeno-associated virus, Aichi virus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immune deficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O'nyong-nyong virus, Orf virus, Oropouche virus, Pichinde virus, poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus and Zika virus.

In another embodiment, the present invention provides the composition comprising the non-stoichiometric adjuvant in combination with one or more immunogenic antigen wherein the immunogenic antigens are associated with specific virus including SARS-COV, SARS-COV2, MERS and MERS-COV.

In another embodiment, the present invention provides the composition comprising the non-stoichiometric adjuvant in combination with one or more immunogenic antigen wherein the immunogenic antigen has been obtained from SARS-COV2 with sequence associated with Spike protein (S).

In another embodiment, the present invention provides the composition comprising the non-stoichiometric adjuvant in combination with one or more immunogenic antigen wherein the immunogenic antigen is that of SARS-COV2 with sequence associated with receptor binding domain (RBD) from the WUHAN variant (Arg319-Phe541).

In another embodiment, the present invention provides the composition comprising the non-stoichiometric adjuvant in combination with one or more immunogenic antigen wherein the immunogenic antigens from other pathogens such as bacteria and fungi causing diseases selected from the group consisting of Tuberculosis, Anthrax, Tetanus, Leptospirosis, Pneumonia, Cholera, Botulism Pseudomonas Infection, MRSA Infection, E. Coli Infection, Meningitis, Gonorrhea, Bubonic Plague, Syphilis.

In another embodiment, the present invention provides the use of the non-stoichiometric adjuvant compounds in immune activating therapeutics wherein the therapies can be against cancer affecting the organs such as brain, liver, kidney, pancreas, prostate, breast, skin, muscle, eyes, thyroid glands, lymphoma, sarcoma, myeloid organs, blood cells, immune cells, bone-marrow, gastro-organs, colon, lungs, bladder, bile duct, adrenal gland, prostate, testis, bone, breast, cervix, oesophagus, head and Neck, lymph nodes, skin, nervous system, ovary, pancreas, soft tissue, stomach, thyroid, thymus, and/or uterus.

In another embodiment, the present invention provides the use of the non-stoichiometric adjuvant compound in combination with other therapy of cancer and infectious or autoimmune diseases such as chemotherapeutics, small molecule inhibitors, immunotherapy, antibody therapy, cytokine therapy, radiation therapy, nuclear medicine, photodynamic therapy, tumour killing field therapy, surgery, anti-viral therapy, ayurvedic, homeopathic and/or traditional medicine therapies.

In another embodiment, the present invention provides the use of the non-stoichiometric adjuvant is targeted to disease-organ, immune organs such as lymph nodes, spleen, payers patch, bone marrow, cancer cells, infectious agents such as virus, bacteria, fungi, using targeting moieties such as antibodies, peptides, small molecules, proteins, aptamers, and/or scFvs.

In another embodiment, the present invention provides the use of the non-stoichiometric adjuvant for sustained release of other therapeutic compounds and enhance the therapeutic effect of radiation therapy, nuclear medicine, immunotherapy.

In another embodiment, the present invention provides the use of the non-stoichiometric adjuvant compound in combination with other cell therapies such as CART-T cell, DC cell vaccine, NK cell therapy, adoptive T cell therapies, stem-cell therapy.

In another embodiment, the present invention provides the use of the non-stoichiometric adjuvant compound in combination with antibody therapies such as immune-checkpoint blockade antibodies against PD1, PDL1, CTLA4, Lag3, IDO, TIM3, growth factor binding antibodies against EGFR, PDGFR, VEGF, VEGFR, Her2/nue, IL6, TNF alpha inhibitor antibody.

In another embodiment, the present invention provides the use of the non-stoichiometric adjuvant compound in combination with therapeutic protein therapy such as for example, GM-CSF, G-CSF, MCSF, FLT3, IL6, type-1 and type-2 IFN, interleukins, chemokines, growth factors.

In another embodiment, the present invention provides the use of the non-stoichiometric adjuvant compound in combination with immune-cell suppressor therapies such as MDSC inhibitors (chemo drugs such as 5FU, gemcitabine, paclitaxel, cell-cycle related kinase inhibitor, CDK inhibitor, Sema4D inhibitor, cisplatin, all-trans retinoic acid (ATRA) arginaze inhibitor, IDO inhibitors, CXCR2 inhibitor, CXCL12/CXCR4 inhibitor, doxycycline, capecitabine, doxorubicine), T-reg inhibitors (cyclophosphamide, steroids), macrophage inhibitors (bisphosphonates, clodronate), antibodies against CD3, CD4, CD11b, and/or CD11C.

In another embodiment, the present invention provides the use of the non-stoichiometric adjuvant compound for mixing with antigens vaccinating veterinary animals and pets.

In another embodiment, the present invention provides the non-stoichiometric micro- and nanostructures as immune-modulatory agents wherein the non-stoichiometric adjuvant can be in the form of large porous microbeads, injectable and/or implantable gels, hydrogels, oral formulations, three-dimensional structures for sustained release of the adjuvant itself and/or antigens, other vaccine components for prolonged periods.

In another embodiment, the present invention provides the non-stoichiometric adjuvant compound having the formula M(n)ₓA(m)_{y} wherein the adjuvant forms an injectable depot at the site of injection.

In another embodiment, the present invention provides a method for the preparation of the non-stoichiometric nano-microparticle adjuvant and associated vaccine composition.

In another embodiment, the present invention provides a method for preparing the immunogenic pharmaceutical composition containing said non-stoichiometric adjuvant compound, antigens, and other immunogenic agents, growth factors, interleukins, chemokines, surfactants, detergents, polymers, stabilizing agents, lipids, carbohydrates, cyto-protectants and storage stabilizers.

In another embodiment, the present invention provides the composition containing the non-stoichiometric adjuvant compound can be administered injected or implanted subcutaneously, intradermal, transdermal, intramuscular, intravenous, intracranial, intrathecal, nasal, and/or oral route.

In another embodiment, the present invention provides a method of generating an immune response in subject injected with the composition by way of activating innate and adaptive immune cells such as DC, macrophages, B cells and T cells and producing specific and non-specific antibodies and T, NK cell, Dendritic cell, macrophage, neutrophil response against the disease condition or antigen used in the vaccine formulation.

In another embodiment, the present invention provides a method of generating an enhanced B cell activation and antibody response in subject injected with the said composition and associated vaccines.

In another embodiment, the present invention provides a method of generating an enhanced T cell response in subject injected with the said composition and associated vaccines.

In another embodiment, the present invention provides a method of generating an enhanced NK cell response in subject injected with the said composition and associated vaccines.

In a preferred embodiment, the present invention provides that the adjuvant is used in combination with an anti-MDSC depletion therapy molecule 5FU or its prodrug; capecitabine for repolarizing M2 macrophages to M1, enhancing cytotoxic T cell infiltration into the tumor and development of memory T cells into lung tumor.

In another embodiment, the present invention provides a method of using the said the non-stoichiometric adjuvant in combination with other vaccines, chemotherapy, immunotherapy, radiation, surgery, and cell therapy, antibody therapy, peptide therapy, hormonal therapy.

In another embodiment, the present invention provides use of the immunogenic pharmaceutical vaccine composition containing non-stoichiometric adjuvant compound for the treatment of cancer in a subject.

In another embodiment, the present invention provides use of the immunogenic pharmaceutical composition containing the non-stoichiometric adjuvant compound for the treatment of infectious disease.

In another embodiment, the present invention provides the use of the immunogenic pharmaceutical composition for the treatment of immune disorders including autoimmunity.

In another embodiment, the present invention provides a kit comprising the immunogenic pharmaceutical vaccine composition along with the instruction manual.

Without limiting the scope of the present invention as described above in any way, the present invention has been further explained through the examples provided below.

### EXAMPLES

The inventive adjuvant nanoparticle or microparticles are synthesized by aqueous-phase reaction between a precursor solution-A containing desired mix of multiple cations at appropriate concentrations and another precursor solution-B containing dissolved salts of anions such as hydroxides, phosphates, sulphates or phosphonates, bisphosphonates, reacted in the presence of surface capping agents and pH modifiers under stirring under normal ambient conditions.

### Example 1: Preparation of M(5)A(1)-1 Adjuvant Nanoparticles: (Ca_{23.39}Mn_{9.15}Zn_{2.38}Al_{1.8}Mg_{0.54})HP_{19.62}O_{43.24}

A calcium-rich, five cations, single anion composition, the cation precursor was formed by mixing aqueous solutions of ~200 mL of 0.05M calcium chloride with 30 mL of 0.1M manganese nitrate, 5 mL of 0.1M zinc chloride, 2.5 mL of 0.25M aluminium chloride, 1 mL of 0.01M magnesium nitrate. To this, 5 mL of 0.05 wt % polysorbate 80 was added and mixed well for 10 minutes. Anion solution-B was prepared by mixing 0.05M di-ammonium hydrogen phosphate blended with 1N ammonia at 3:2 ratio. The precipitation reaction was started by drop-wise (10-20 uL/sec) addition of solution-B to solution-A under magnetic stirring.

After the complete addition of solution-B, the pH was adjusted to 7-8 by adding the additional volume of solution-B to solution-A. A white precipitate was formed when pH was near 5 which thickened towards pH 7-8 and the same was stirred for 02 hrs and allowed to age for 12-16 Hrs before washing. The precipitate was washed by centrifugation at 5000 rpm for 10 minutes, repeated 5-8 times with MilliQ water and the pellet was re-dispersed into endotoxin-free water or lyophilized for vaccine applications.

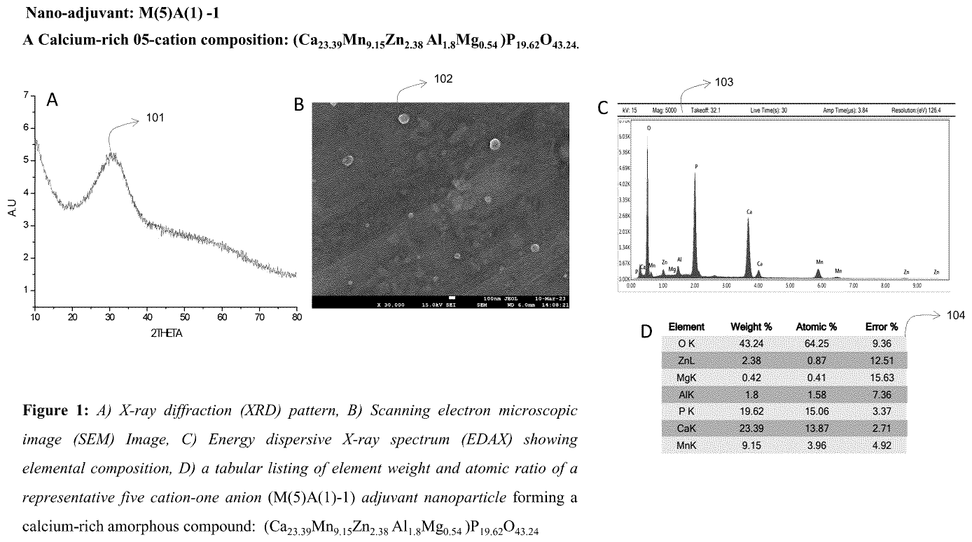

The XRD pattern, 101 SEM image, 102, and elemental composition quantified using EDAX analysis, 103,104 are shown in FIG. 1, which confirm the formation of (Ca_{23.39}Mn_{9.15}Zn_{2.38}Al_{1.8}Mg_{0.54}) P_{19.62}O_{43.24} with subscript showing the weight percentage of each of the elements. Presence and concentration of hydrogen was not detectable by EDAX.

### Example 2: Preparation of M(5)A(1)-2: Zinc-Rich 05 Cations, Single Anion Adjuvant Nanoparticle (Zn_{9.72}Al_{4.7}Ca_{4.08}Mn_{3.97}Mg_{0.16}) HP_{10.85}O_{53.79}

Another specific zinc-rich, five cations, single-anion composition was prepared. The cation precursor solution was formed by mixing aqueous solutions of 100 ml, 0.05M calcium chloride with 16.5 mL of 0.1M manganese nitrate, 15.7 mL of 0.1M zinc chloride, 2.5 mL of 0.25M aluminium chloride, and 0.5 mL of 0.01M magnesium nitrate. To this 5 mL of 0.05 wt % polysorbate 80 was added and mixed well. The mixture is allowed to stir for 10 minutes.

Anion solution-B was prepared by mixing 25 mL of di-ammonium hydrogen phosphate blended with 1N ammonium hydroxide at 3:2 ratio, pH 12-13. The precipitation reaction was started by dropwise (10-20 uL/sec) addition of solution-B to solution-A under magnetic stirring. The reaction continued till the pH of the reaction medium turned 7-8 with a white precipitate started forming by pH 5.

After complete addition, the final pH was adjusted to 7-8 by adding the required volume of solution-B. A white colloidal precipitate formed was stirred for 02 hrs and allowed to age for 12-16 Hrs. Then the precipitate was washed by centrifugation at 5000 rpm for 10 minutes, repeated 5-8 times with MilliQ water and the pellet is re-dispersed into endotoxin-free water or lyophilized for applications.

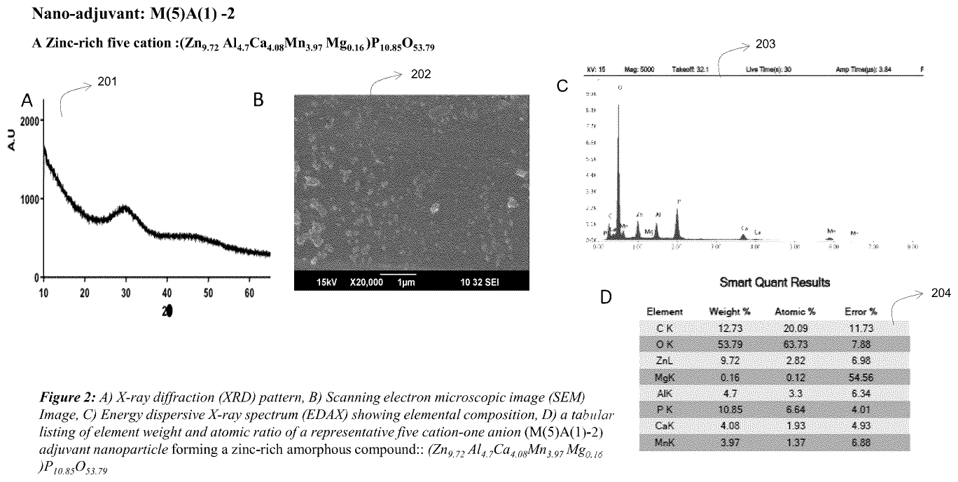

The XRD pattern, 201, SEM image, 202, and elemental composition quantified using EDAX analysis, 203, 204 shown in FIG. 2 confirm the formation of (Zn_{9.72}Al_{4.7}Ca_{4.08}Mn_{3.97}Mg_{0.16}) HP_{10.85}O_{53.79}. The concentration of hydrogen was not measurable by EDAX.

### Example 3: Preparation of M(4)A(1)-1: Manganese-Rich Four Cation Adjuvant Nanoparticles: (Mn_{27.63}Zn_{5.9}Al_{3.71}Mg_{0.54})HP_{15.93}O_{43.3}

A specific manganese-rich, four-cation hydrogen phosphate composition was prepared. The cation precursor solution was formed by mixing aqueous solutions of 100 mL, 0.1M manganese nitrate with 10 mL of 0.1M zinc chloride, 2.5 mL of 0.25M aluminum chloride, and 1 mL of 0.01M magnesium nitrate. To this, 5 mL of 0.05 wt % polysorbate 80 was added and mixed well. The mixture was allowed to stir for 10 minutes.

Anion solution-B was prepared by mixing 25-mL of di-ammonium hydrogen phosphate blended with 1N ammonia at 3:2 ratio, pH 12-13. The precipitation reaction was started by dropwise (10-20 uL/sec) addition of solution-B to solution-A under magnetic stirring at ~23 degree Celsius.

After complete addition, the final pH was adjusted to 7-8 by adding required volume of solution-B. A white colloidal precipitate was formed from pH 5-6 which was thickened over pH 7-8 and continued to stir for 02 hrs and aged for 12-16 Hrs. The precipitate was washed by centrifugation at 5000 rpm for 10 minutes, repeated 5-8 times with MilliQ water and the pellet is re-dispersed into endotoxin-free water or lyophilized for applications.

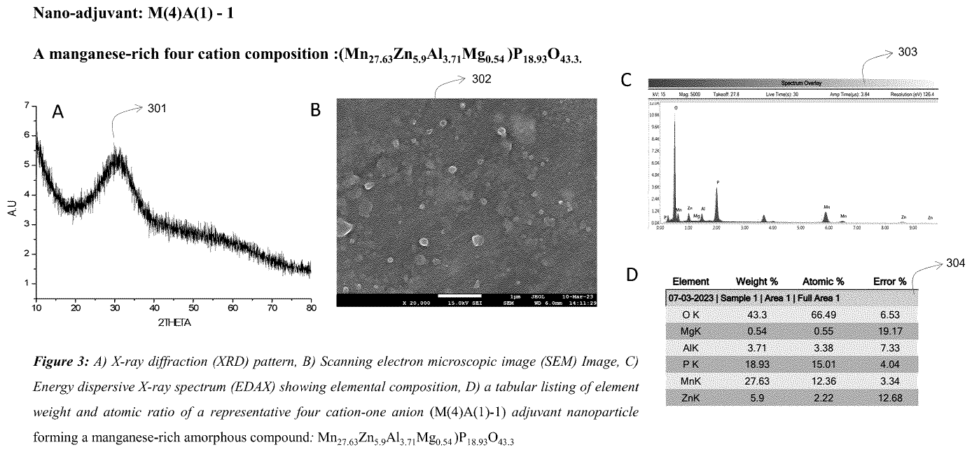

The XRD pattern, 301, SEM image, 302, and elemental analysis using EDAX, 303 and 304 are shown in FIG. 3 which confirms the formation of (Mn_{27.63}Zn_{5.9}Al_{3.71}Mg_{0.54})P_{18.93}O_{43.3}

### Example 4: Preparation of M(4)A(1)-2: Zinc-Rich, Four Cation, Two Anion Adjuvant Nanoparticles: (Zn_{11.94}Mn_{6.61}Al_{6.39}Ca_{6.39})HP_{18.93}O_{43.3}

A zinc-rich, four cation hydroxy-hydrogen phosphate composition was prepared. The cation precursor solution was formed by mixing aqueous solutions of 100 mL, 0.1M manganese nitrate with 10 mL of 0.1M zinc chloride, 2.5 mL of 0.25M aluminum chloride, and 1 mL of 0.01M magnesium nitrate. To this 5 mL of 0.05 wt % polysorbate 80 was added and mixed well. The mixture was allowed to stir for 10 minutes.

Anion solution-B was prepared by mixing 25-mL of di-ammonium hydrogen phosphate blended with 1N ammonia and 0.5 N sodium hydroxide at 3:2:1 ratio. The precipitation reaction was started by dropwise (10-20 uL/sec) addition of solution-B to solution-A under magnetic stirring, till the pH of the final reaction medium reaches 9-10. A white colloidal gel like precipitate was formed from pH 6-7 which was thickened over pH 7-8 and continued to stir for 02 hrs and aged for 12-16 Hrs. The precipitate was washed by centrifugation at 5000 rpm for 10 minutes, repeated 5-8 times with MilliQ water and the pellet is re-dispersed into endotoxin free water or lyophilized for applications.

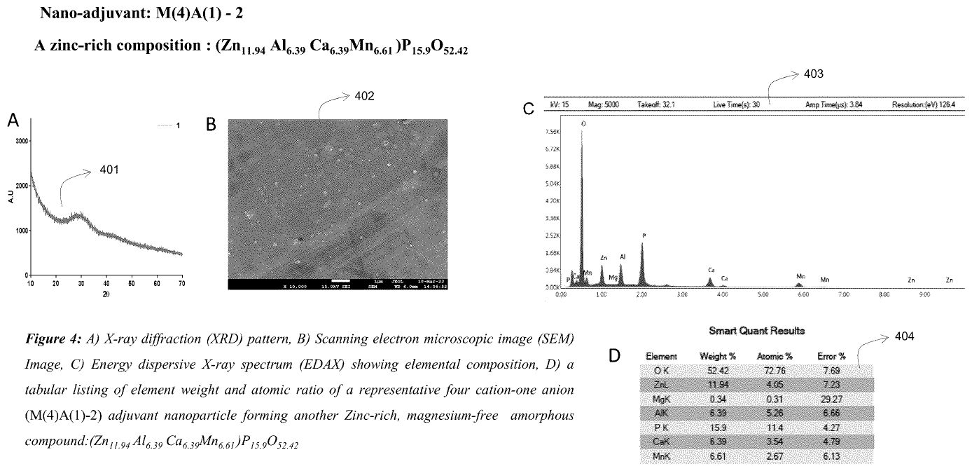

The XRD pattern, 401, SEM images, 402, and elemental analysis using EDAX, 403 and tabular column indicating quantitative estimate of each elements, 404 are shown in FIG. 4 which confirms the formation of (Zn_{11.94}Mn_{6.61}Al_{6.39}Ca_{6.39})(OH)HP_{15.93}O_{43.3}

### Example 5: Preparation of M(4)A(1)-3: Manganese-Rich, Four Cation, Two Anion Adjuvant Nanoparticles: (Mn_{13.94}Zn_{0.86}Al_{5.98}Mg_{0.52})HP_{19.77}O_{58.94}

A manganese-rich, four cation, two anion nanoparticle composition was prepared. The cation precursor solution was formed by mixing aqueous solutions of 150 mL, 0.1M manganese nitrate with 1 mL of 0.1M zinc chloride, 2.5 mL of 0.25M aluminum chloride, and 1 mL of 0.1 magnesium nitrate. To this 5 mL of 0.05 wt % polysorbate 80 was added and mixed well. The mixture was allowed to stir for 10 minutes.

Anion solution-B was prepared by mixing 25-mL of di-ammonium hydrogen phosphate blended with 1N ammonia and 0.5 N sodium hydroxide at 3:2:1 ratio. The precipitation reaction was started by dropwise (10-20 uL/sec) addition of solution-B to solution-A under magnetic stirring, till the pH of the final reaction medium reaches 9-10. A white colloidal gel like precipitate was formed from pH 6-7 which was thickened over pH 7-8 and continued to stir for 02 hrs and aged for 12-16 Hrs. The precipitate was washed by centrifugation at 5000 rpm for 10 minutes, repeated 5-8 times with MilliQ water and the pellet is re-dispersed into endotoxin free water or lyophilized for applications.

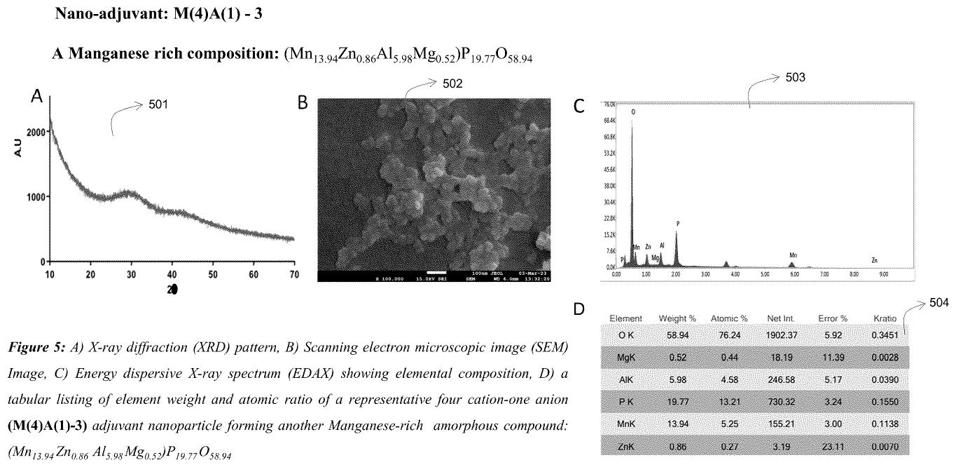

The XRD pattern 501, SEM images, 502, and elemental analysis using EDAX, 503 and tabular column on elements detected by EDAX, 504, are as shown in FIG. 5, which confirms the formation of (Mn_{13.94}Zn_{0.86}Al_{5.98}Mg_{0.52})(OH)HP_{19.77}O_{58.94}

### Example 6: Preparation of M(4)A(1)-4: Manganese-Rich, Four Cation, Two Anion Adjuvant Microparticles: (Mn_{22.19}Zn_{9.03}Al_{4.99}Mg_{0.19})HP_{16.65}O_{37.06}

A manganese-rich, four cation, two anion microparticle composition was prepared. The cation precursor solution was formed by mixing aqueous solutions of 200 mL, 0.1M manganese nitrate with 20 mL of 0.1M zinc chloride, 2.5 mL of 0.25M aluminum chloride, and 0.5 mL of 0.1 magnesium nitrate. Here, no surfactant was added to control the particle size. The cation mixture was allowed to stir for 10 minutes.

Anion solution-B was prepared by mixing 25-mL of di-ammonium hydrogen phosphate blended with 1N ammonia and 0.5 N sodium hydroxide at 3:2:1 ratio. The precipitation reaction was started by dropwise (10-20 uL/sec) addition of solution-B to solution-A under magnetic stirring, till the pH of the final reaction medium reaches 9-10. A white colloidal gel like precipitate was formed from pH 6-7 which was thickened over pH 7-8 and continued to stir for 02 hrs and aged for 24 Hrs. The precipitate was washed by centrifugation at 5000 rpm for 10 minutes, repeated 5-8 times with MilliQ water and the pellet is re-dispersed into endotoxin free water or lyophilized for applications.

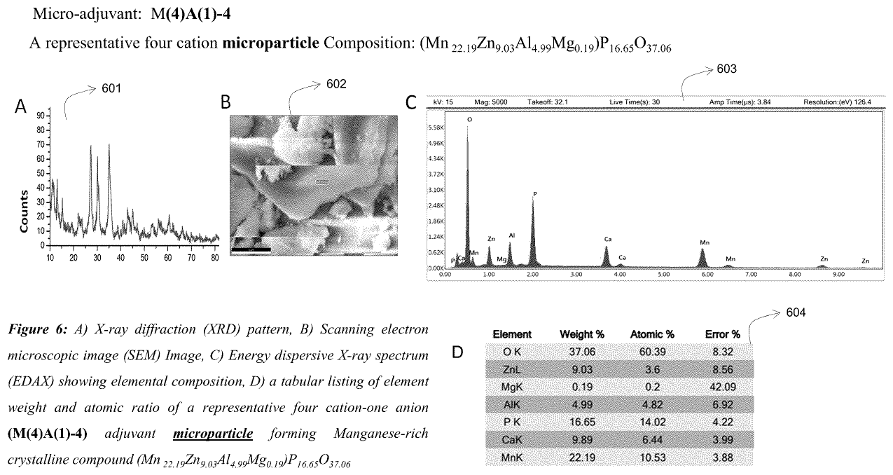

The XRD pattern, 601, indicating polycrystalline bulk material, SEM images, 602, showing platelike structures and elemental analysis using EDAX, 603, as shown in FIG. 6 confirms the formation of micron sized adjuvant composition (Mn_{22.19}Zn_{9.03}Al_{4.99}Mg_{0.19}) HP_{16.65}O_{37.06.}

### Example 7: Preparation of M(5)A(3)-1: Manganese-Calcium-Zinc-Aluminum-Magnesium-Di-Hydrogen Phosphate-Alendronate:(Mn_{33.9}Ca_{3.7}Zn_{5.5}Al_{4.14}Mg_{0.12})-HPO4-(OP)₂—(OH)₄—COH—(CH₂)₃NH₂)

A manganese-rich, five-cations and three anions (dihydrogen-phosphate-hydroxy-alendronate) composition was prepared. The cation precursor solution-A was formed by mixing aqueous solutions of 50 mL 0.05M calcium chloride with 100 mL of 0.1M manganese nitrate, 5 mL of 0.1M zinc chloride, 5 mL of 0.25M aluminium chloride, 0.5 mL magnesium nitrate (0.01M). To this, 6 mL aqueous solution of 25 mg/mL Alendronate ((OP)₂—(OH)₄—COH—(CH₂)₃NH₂)) was added followed by 5 mL of 0.05 wt % polysorbate-80 and mixed well for 10 minutes. This resulted into the formation of a multi-cation-alendronate intermediate.

Further, other anions in solution-B containing 0.05M di-ammonium hydrogen phosphate blended with 1N ammonium hydroxide at 3:2 ratio (25-30 mL) was added dropwise (10-20 uL/sec) to solution-A under magnetic stirring. After complete addition of solution-B, the pH was adjusted to 9-10 by adding additional volume of solution-B to solution-A. A white gel-like precipitate formed was stirred for 02 hrs and the precipitate was washed by centrifugation at 5000 rpm for 10 minutes, repeated 5-8 times with MilliQ water and re-dispersed into endotoxin-free water. This nanoparticle dispersion was again treated with a surface layer of alendronate (OP)₂—(OH)₄—COH—(CH₂)₃NH₂) for a better coordination of bisphosphonate ions with metal cations on the surface of nanoparticles. Unreacted bisphosphonate was removed by centrifugation and washed or lyophilized for vaccine applications.

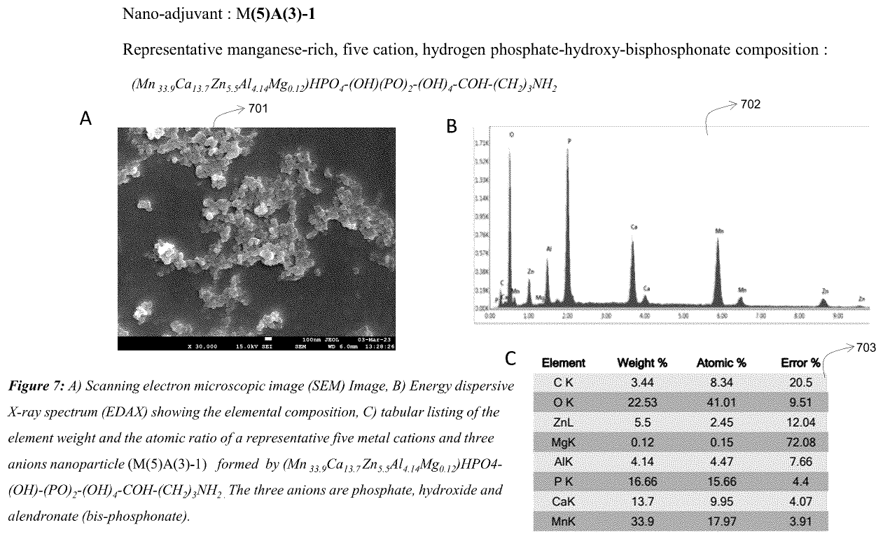

The SEM image, 701, and elemental composition quantified using EDAX analysis, 702, and tabular column of elements as detected by EDS, 703 as shown in FIG. 7 confirms the formation of (Mn_{33.9}Ca_{13.7}Zn_{5.5}Al_{4.14}Mg_{0.2})-HPO4-(OP)₂—(OH)₄—COH—(CH₂)₃NH₂).

### Example 8. Preparation of M(5)A(3)-2: Ca—Mn rich (Mn_{6.61}Ca_{6.39} Zn_{11.94}Al_{6.36}Mg_{0.34}) HPO4-(OH)—(PO)₂—(OH)₄—COH—(CH₂)₃NH₂ Nanoparticles

A calcium-manganese rich (~6 wt % each), five cation and three anion (dihydrogen-phosphate, hydroxyl, bis-phosphonate) nanoparticle composition was prepared. The cation-bisphosphonate precursor solution-A was formed by mixing aqueous solutions of ~100 mL 0.05M calcium chloride mixed with 100 mL of 0.0.05M manganese nitrate, 5 mL of 0.1M zinc chloride, 2.5 mL of 0.25M aluminum chloride, 0.5 mL magnesium nitrate (0.1M). To this, 6 mL aqueous solution of 25 mg/mL Alendronate ((OP)₂—(OH)₄—COH—(CH₂)₃NH₂)) was added to form a (Ca—Mn—Zn—Al—Mg)—(OP)₂—(OH)₄—COH—(CH₂)₃NH₂) intermediate by way of coordination of phosphate group with metal ions, but with no visible precipitate. In the next step, 5 mL of 0.05 wt % polysorbate-80 was added and mixed well for 10 minutes.

Further, other anion solution-B containing 0.05M di-ammonium hydrogen phosphate blended with 1N ammonium hydroxide at 3:2 ratio (25-30 mL) was added dropwise (10-20 uL/sec) to cation-alendronate intermediate under magnetic stirring to precipitate the multi-cation-dihydrogen-phosphate-hydroxyl-alendronate as a white precipitate. After complete addition of anion solution-B, the pH was adjusted to 7-8 by adding additional volume of solution-B. The white precipitate formed was stirred for 02 hrs and allowed to age for 12-16 Hrs. Then the precipitate was washed by centrifugation at 5000 rpm for 10 minutes, repeated 5-8 times with MilliQ water and the pellet was re-dispersed into endotoxin-free water. This nanoparticle dispersion was again treated with an additional surface layer of alendronate; (OP)₂—(OH)₄—COH—(CH₂)₃NH₂) for a secondary bisphosphonate coordination with metal cations on the surface of nanoparticles. Unreacted bisphosphonate was removed by centrifugation and washing.

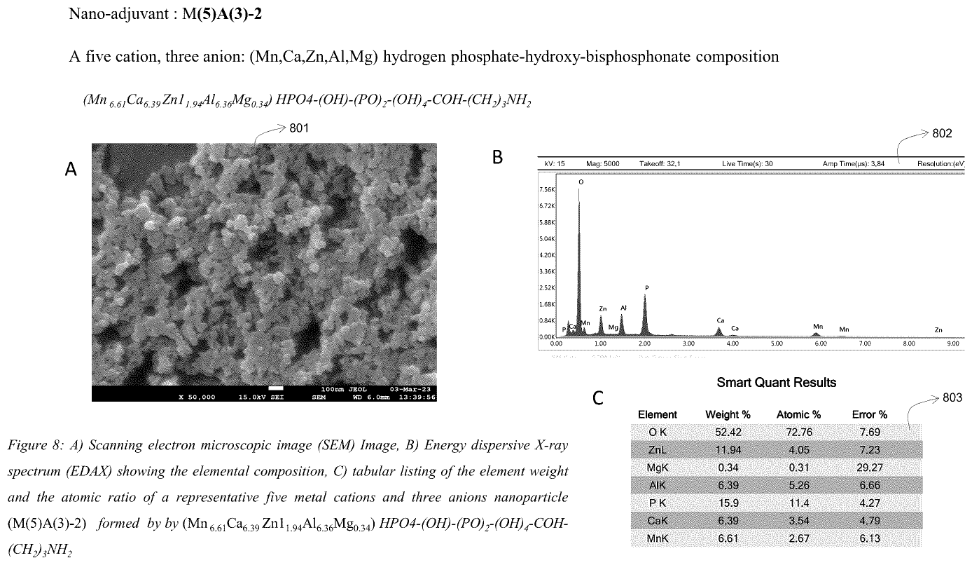

The SEM image, 801, elemental analysis using EDAX pattern, 802, and tabular column of elements present in sample, 803, as shown in FIG. 8 confirms the formation of Mn 6.61Ca_{6.39} Zn_{11.94}Al_{6.36}Mg_{0.34}.

### Example 9. Preparation of M(5)A(3)-3: Calcium-Rich (Ca,Mn,Zn,Al,Mg)-Dihydrogen Phosphate-Hydroxyl-Alendronate Nanoparticles: (Ca_{22.21}Mn_{5.7}Zn_{2.24}Al_{1.69}Mg_{0.39})HPO4-(OH)—(OP)₂—(OH)₄—COH—(CH₂)₃NH₂)

A calcium-rich (22.21 wt %) five cation and three anion (dihydrogen-phosphate, hydroxyl, bis-phosphonate groups) composition was prepared. The cation-bisphosphonate precursor solution-A was formed by mixing aqueous solutions of ~200 mL 0.05M calcium chloride mixed with 10 mL of 0.1M manganese nitrate, 5 mL of 0.1M zinc chloride, 2.5 mL of 0.25M aluminum chloride, 0.5 mL magnesium nitrate (0.1M). To this, 6 mL aqueous solution of 25 mg/mL Alendronate ((OP)₂—(OH)₄—COH—(CH₂)₃NH₂)) was added to form a (Ca—Mn—Zn—Al—Mg)—(OP)₂—(OH)₄—COH—(CH₂)₃NH₂) intermediate by way of coordination of phosphate group with metal ions, but with no visible precipitate. In the next step, 5 mL of 0.05 wt % polysorbate-80 was added and mixed well for 10 minutes.

Further, other anion solution-B containing 0.05M di-ammonium hydrogen phosphate blended with 1N ammonium hydroxide at 3:2 ratio (25-30 mL) was added dropwise (10-20 uL/sec) to cation-alendronate intermediate under magnetic stirring to precipitate the multi-cation-dihydrogen-phosphate-hydroxyl-alendronate as a white precipitate.

After complete addition of anion solution-B, the pH was adjusted to 7-8 by adding additional volume of solution-B. The white precipitate formed was stirred for 02 hrs and allowed to age for 12-16 Hrs. Then the precipitate was washed by centrifugation at 5000 rpm for 10 minutes, repeated 5-8 times with MilliQ water and the pellet was re-dispersed into endotoxin-free water. This nanoparticle dispersion may be again treated with an additional surface layer of alendronate; (OP)₂—(OH)₄—COH—(CH₂)₃NH₂) for a secondary bisphosphonate coordination with metal cations on the surface of nanoparticles. Unreacted bisphosphonate is removed by centrifugation and washing.

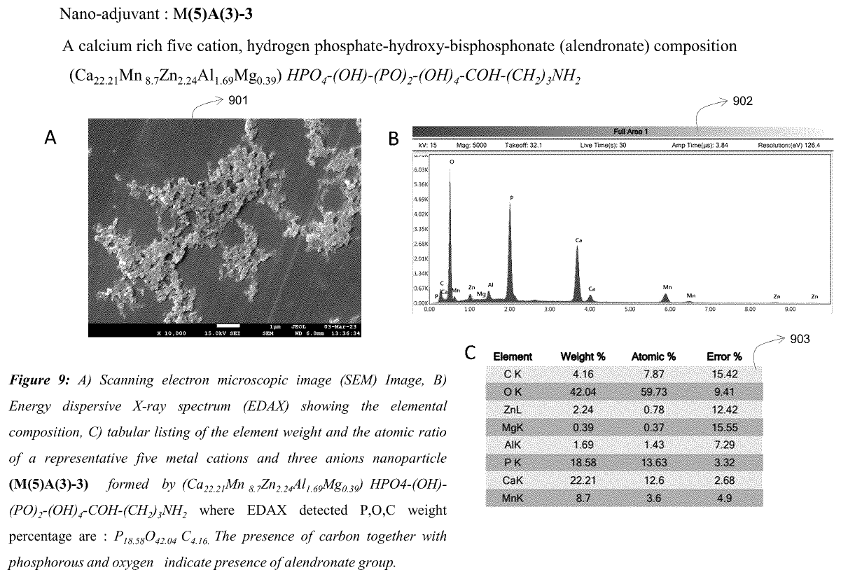

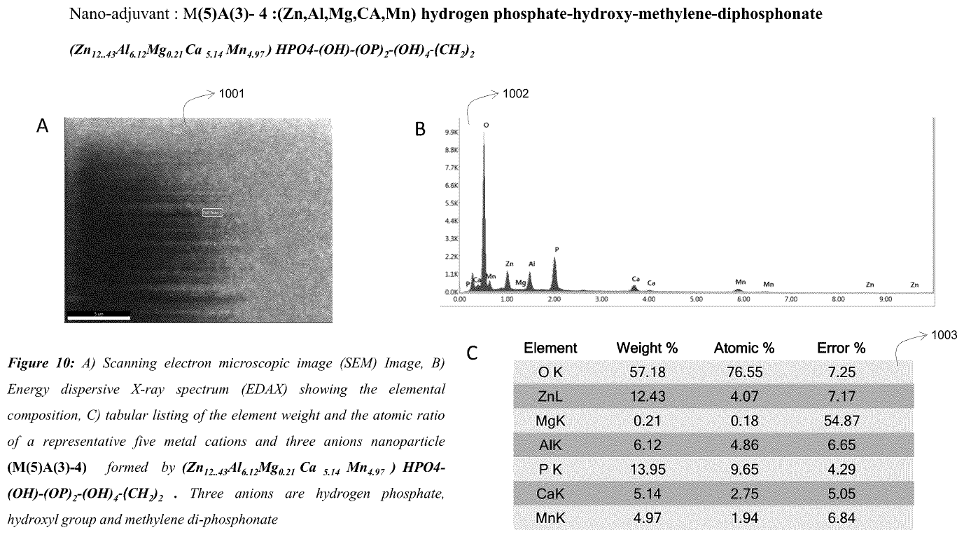

The SEM image, 901, elemental analysis using EDAX, 902, and tabular column of elements, 903, in FIG. 9 confirms formation of (Ca_{22.21}Mn_{8.7}Zn_{2.24}Al_{1.69}Mg_{0.39})

### Example 10. Preparation of M(5)A(3)-4: (Mn,Zn,Al,Mg)-Hydrogen Phosphate-Hydroxyl-Methylene Diphosphonate Nanoparticles: (Zn_{12.43}Al_{6.12}Mg_{0.21} Ca_{5.14} Mn_{4.97}) HPO4-(OH)—(OP)₂—(OH)₄—(CH₂)₂

A Manganese rich (22.19 wt %), four cation and three anion (dihydrogen-phosphate, hydroxyl, methylene di-phosphonate (MDP)) composition was prepared. The cation-bisphosphonate precursor solution-A was formed by mixing aqueous solutions of ~100 mL of 0.1M manganese nitrate, 5 mL of 0.1M zinc chloride, 2.5 mL of 0.25M aluminium chloride, 0.5 mL magnesium nitrate (0.01M) and 6 mL aqueous solution of 25 mg/mL methylene diphosphonate ((OP)₂—(OH)₄—(CH2)2) to form a (Ca—Mn—Zn—Al—Mg)—((OP)₂—(OH)₄—(CH₂)₂) intermediate by way of coordination of diphosphate group with metal ions (Mn—Zn—Al—Mg), but with no visible precipitate. In the next step, 5 mL of 0.05 wt % polysorbate-80 was added and mixed well for 10 minutes.

Further, other anion solution-B containing 0.05M di-ammonium hydrogen phosphate blended with 1N ammonium hydroxide and 0.5N NaOH at 3:2:1 ratio (15-20 mL), added dropwise (10-20 uL/sec) to cation-alendronate intermediate under magnetic stirring to precipitate the multi-cation-dihydrogen-phosphate-hydroxyl-mythylene-di-phosphonate as a white precipitate.

After complete addition of anion solution-B, the pH was adjusted to 7-8 by adding additional volume of solution-B. The white precipitate formed was stirred for 02 hrs and allowed to age for 12-16 Hrs. Then the precipitate was washed by centrifugation at 5000 rpm for 10 minutes, repeated 5-8 times with MilliQ water and the pellet was re-dispersed into endotoxin-free water. This nanoparticle dispersion will be again treated with an additional surface layer of MDP; for a secondary bisphosphonate coordination with metal cations on the surface of nanoparticles, leading to pH 5-6. Unreacted bisphosphonate is removed by centrifugation and washing.

SEM image, 1001, and EDAX pattern, 1002, and tabular column of elements, 1003 in FIG. 9 confirms the formation of (Zn_{12.43}Al_{6.12}Mg_{0.21} Ca_{5.14} Mn_{4.97}) HPO4-(OH)—(OP)₂—(OH)₄—(CH₂)₂

### Example 11: Preparation of Aluminium-Rich M(4)A(2)-1: Aluminium-Magnesium-Manganese-Zinc-Hydroxy-Hydrogen Phosphate (Al_{10.41}Zn_{2.28} Mn_{0.85} Mg_{0.59})(OH)HP_{44.26}O_{41.93}. Nanoparticles

An Aluminium-magnesium-dihydrogen-phosphate-hydroxide composition was prepared. The four-cation precursor was formed by mixing aqueous solutions of 200 mL 0.1M aluminium chloride, 0.01M, 25 mL magnesium nitrate, 50 mL 0.1M zinc chloride, 10 mL 0.01M magnesium sulphate. To this 5 mL of 0.05 wt % polysorbate 80 was added and mixed well for 10 minutes.

Anion solution-B was prepared by mixing 0.05M di-ammonium hydrogen phosphate blended with 1N ammonium hydroxide at 3:2 ratio. The precipitation reaction was started by dropwise (10-20 uL/sec) addition of solution-B to solution-A under magnetic stirring until the pH of the reaction medium reaches to 7-8. A white precipitate formed was stirred for 02 hrs. The precipitate was washed by centrifugation at 5000 rpm for 10 minutes, repeated 5-8 times with MilliQ water and the pellet was re-dispersed into endotoxin-free water or lyophilized for vaccine applications.

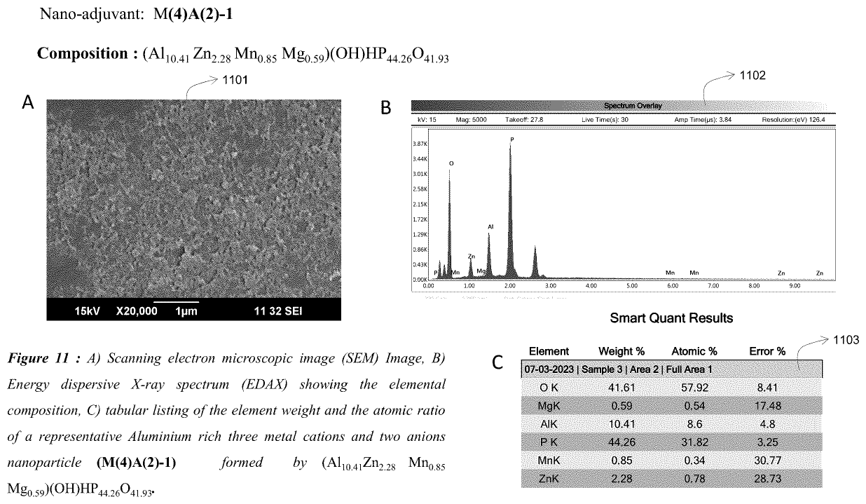

FIG. 11 depicts the SEM image, 1011, and EDAX pattern, 1102, 1103, of the sample (Al_{10.41}Zn_{2.28} Mn_{0.85} Mg_{0.59})(OH)HP_{44.26}O_{41.93},

### Example 12: Preparation of Aluminium-Rich M(2)A(3)-1: Aluminum-Manganese Hydroxy-Hydrogen Phosphate-Alendronate; Al_{27.07}Mn_{11.5}(OH)HP_{14.98}O_{46.03}—(OP)₂—(OH)₄—COH—(CH₂)₃NH₂)

An Aluminium-manganese-hydroxy-hydrogen-phosphate-alendronate composition was prepared. The two-cation precursor-alendronate mixture (Solution-A) was formed by mixing aqueous solutions of 250 mL 0.1M aluminum chloride, and 50 mL of 0.1M Manganese chloride. To this, 10 mL, 25 mg/mL alendronate was added to form a soluble intermediate of (Al,Mn)—(OP)₂—(OH)₄—(CH₂)₂. Further, 5 mL of 0.05 wt % polysorbate 80 was added to this intermediate and mixed well for 10 minutes.

Anion solution-B was prepared by mixing 0.05M di-ammonium hydrogen phosphate blended with 1N ammonium hydroxide at 3:2 ratio. The precipitation reaction was started by dropwise (10-20 uL/sec) addition of solution-B to solution-A under magnetic stirring till the reaction pH reached 7-8. A white precipitate formed was stirred for 02 hrs. The precipitate was washed by centrifugation at 5000 rpm for 10 minutes, repeated 5-8 times with MilliQ water and the pellet was re-dispersed into endotoxin-free water or lyophilized for vaccine applications.

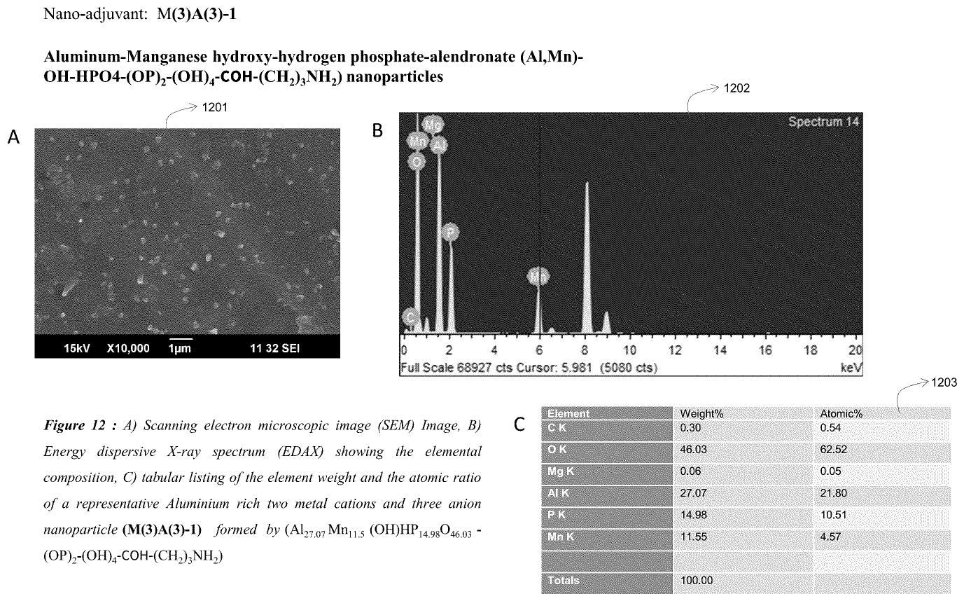

FIG. 12 depicts the SEM image, 1201, and EDAX pattern, 1202 of nanoparticles formed.

### Example 13: Preparation of M(4)A(2)-1: (Ca,Mg,Mn,Zn) Sulphate-Methylene-Diphosphonate Microcrystal Adjuvant

A 4-cation sulphate-methylene-diphosphonate microcrystal adjuvant composition was prepared. The four-cation precursor containing Ca, Mg, Mn, Zn and methylene diphophonic acid (MDP) was formed by mixing aqueous solutions of 250 mL 0.1M Calcium sulphate, and 250 mL of 0.1M Magnesium chloride, 50 mL Manganese chloride 0.1M, 50 mL Zinc chloride 50 mL were mixed. To this, 20 mL, 25 mg/mL MDP was added to form a soluble intermediate of (CA,Mg,Mn,Zn)—(OP)₂—(OH)₄—(CH₂)₂. Further, 5 mL of 0.05 wt % polysorbate 80 was added to this intermediate and mixed well for 10 minutes. The precipitation reaction was started within 05 minutes of mixing calcium sulphate with magnesium chloride in presence of Zn and Mn and MDP A white needular precipitate formed was stirred for 02 hrs and washed by centrifugation at 5000 rpm for 10 minutes, repeated 5-8 times with MilliQ water and the pellet was re-dispersed into endotoxin-free water or lyophilized for vaccine applications.

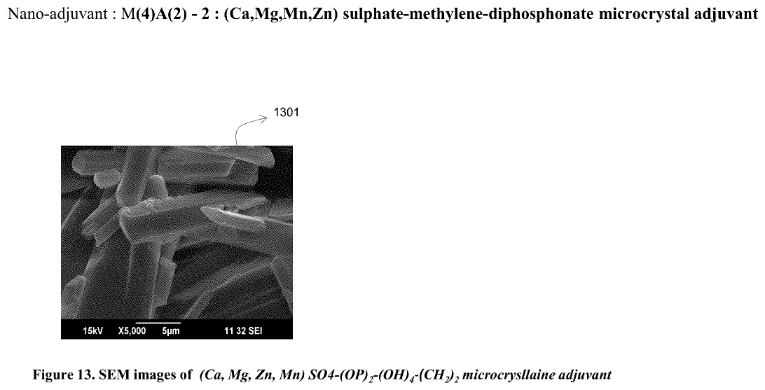

FIG. 13 depicts the SEM, 1301, image of micro needular adjuvant particles formed particles formed

### Example 14: Preparation of M(4)A(2): Aluminium Rich, Four Cation, Two Anion Adjuvant Nanoparticles: Al_{83.9} Zn_{4.89} Mg_{7.59}Ca_{3.42})HPO₄—(OP)₂—(OH)₄—COH—(CH₂)₃NH₂

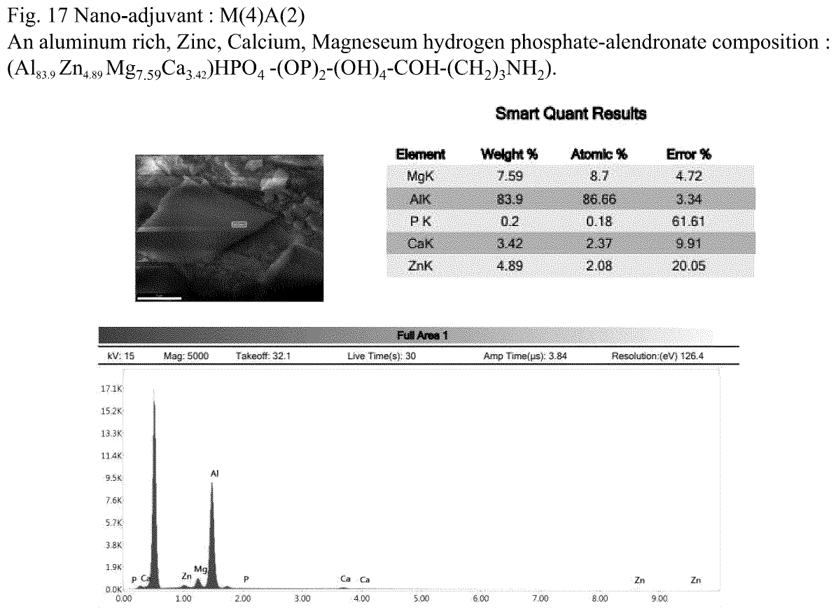

An Al rich, four cation hydrogen phosphate-alendronate composition was prepared. by reacting aqueous solutions of 200 mL, 0.1 Aluminium chloride with 0.1M zinc chloride, 2.5 mL and 1 mL of 0.01M magnesium nitrate, 1 mL of 0.01M calcium chloride. The mixture was allowed to stir for 10 minutes. To this 1 ml of 25 mg/mL alendronate solution was added to form a metal ions alendronate complex Anion solution-B was prepared by mixing 25-mL of di-ammonium hydrogen phosphate blended with 1N ammonia and 0.5 N sodium hydroxide at 3:2:1 ratio. The precipitation reaction was started by dropwise (10-20 uL/sec) addition of solution-B to solution-A under magnetic stirring, till the pH of the final reaction medium reaches 9-10. A white colloidal gel like precipitate was formed from pH 6-7 which was thickened over pH 7-8 and continued to stir for 02 hrs and aged for 12-16 Hrs. The precipitate was washed by centrifugation at 5000 rpm for 10 minutes, repeated 5-8 times with MilliQ water and the pellet is re-dispersed into endotoxin free water or lyophilized for applications. EDAX pattern and SEM image of the thus formed adjuvant particle is shown in FIG. 17.

### Example 14: Dynamic Light Scattering (DLS) Analysis

Dynamic light scattering (DLS) analysis was done to examine the particle size range of various particles formed. The size distribution of the M(n)ₓA(m)_{y} was also determined by Dynamic Light Scattering using ZETASIZER Nanoseries. The sample was diluted to 1:1000 and 1 ml of this homogenised sample was transferred into the cuvette and placed for reading.

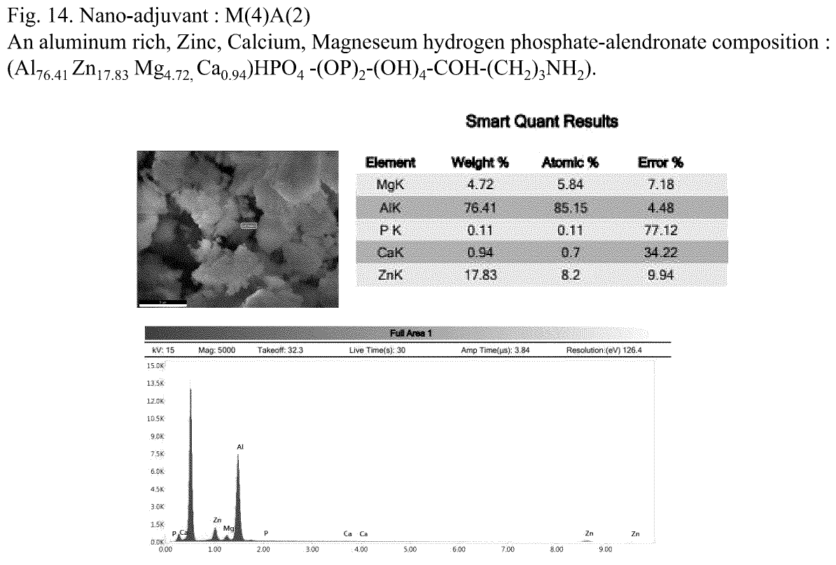

FIG. 14 depicts two size of Mₓ(n)A_{y}(m) nanoparticle adjuvants A) 100-200 nm range (1401) and B) 800-1600 nm range (1402).

### Example 15: In Vitro Biocompatibility of a Representative Adjuvant Nanoparticles M(5)A(3)-1 was Tested in Dendritic Cells

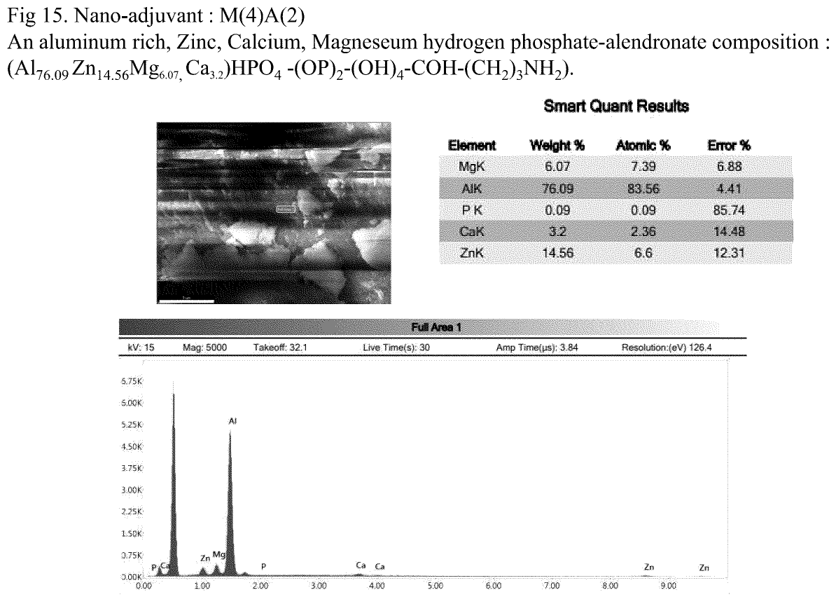

For the MTT assay, 2×10 4 Jaws II cells were seeded in a 96 well plate and incubated at 37° C. for 24 hours. After incubation, the cells were treated with nanoparticles in concentrations ranging from 10 μg/ml-200 μg/ml. After treatment, the cells were incubated at 37° C. for 24 hr. After 24 hours of treatment, MTT was added to each well to make final concentration of 10% (Perform under dark condition) and incubate the plate for 4 hours at 37° C. for the formation of formazan crystals. After incubation, solubilisation buffer was added to each well and formazan crystals were dissolved by thorough mixing using a pipette. Measured the absorbance at 570 and 660 nm using microplate reader and data is depicted in FIG. 15. The data, 1501, suggest no toxicity by the nanoparticles to immune cells represented by dendritic cell line, up to a tested high concentration of 200 ug/mL.

### Example 16: Preparation of SARS COV2 Vaccine Using M(n)A(m) Adjuvant Nanoparticle and Vaccination in Mice Models

For the preparation of vaccine with M(5)A(3)-1 adjuvant nanoparticles, RBD sequence- (Arg319-Phe541) from SARS-COV2 virus was taken. The antigen has the following sequence ID.:

RVQPTESIVRFPNITNLCPFGEVFNATRFASVYAWNRKRISNCVAD
YSVLYNSASFSTFKCYGVSPTKLNDLCFTNVYADSFVIRGDEVRQ
IAPGQTGKIADYNYKLPDDFTGCVIAWNSNNLDSKVGGNYNYLYR
LFRKSNLKPFERDISTEIYQAGSTPCNGVEGENCYFPLQSYGFQP
TNGVGYQPYRVVVLSFELLHAPATVCGPKKSTNLVKNKCVNF

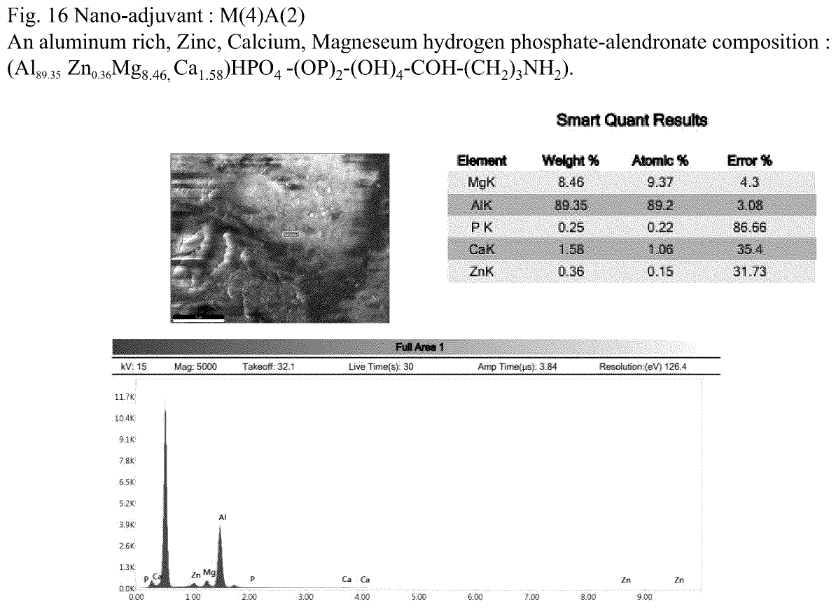

The preparation of M(5)A(3)-1 adjuvant nanoparticle-based SARS-COV2 (S-RBD peptide) vaccine, viz. M(5)A(3)-1-RBD1, negatively charges M(5)A(3)-1 nanoparticle (zeta potential: −12 to −16 mV) and positively charged peptide (S-RBD peptide, +9 to +6 mV) were reacted by first probe sonicating 1 mg of pre-formed adjuvant nanoparticle at 30% amplitude for 3 min in endotoxin-free water for uniform dispersion. The sonicated nanoparticles were then pipette mixed with 25 ug of S-RBD peptide and left overnight at 4° C. in rotating spinner at 25 RPM for surface adsorption of antigen to obtain the SARS COV2 Vaccine using adjuvant nanoparticle (FIG. 16).

For animal vaccine study a dose of 10 mg/kg nano-vaccine (1601) particles corresponding to 5 ug peptide antigen was injected intramuscular in mice models as per example 16.

### Example 17: Preparation of Cancer Vaccine Using Adjuvant Nanoparticle

A representative Cancer (melanoma) vaccine was prepared using M(4)A(2)-1: (Ca, Mg, Zn,Mn)SO4-(OP)₂—(OH)₄—(CH₂)₂ adjuvant and melanoma peptides (TRP2 & gp100) blended with TLR agonists (CpG and Poly IC), and Growth factor mGM-CSF). The nanoparticle adjuvant melanoma vaccine was prepared by surface modification of nanoparticle with a cationic carrier peptide (protamine-sulphate (PS)) and reacted with a cocktail of cancer (melanoma) peptide in the presence of a surfactant.

The peptide cocktail consists of a mixture of total tumor antigen (TTA) 500 ug and TRP2 and GP100 antigen 100 ug each (Genscript, USA) in 0.001% non-ionic surfactant (Saponin, sigma Aldrich). Briefly the vaccine formulation was prepared by probe sonicating 2 mg pre-formed nanoparticle at 30% amplitude for 3 min for uniform dispersion and then interacted with 500 ug cationic peptide (PS) by 30 s probe sonication at 20% amplitude. The surface modified nanoparticle is then interacted with the cocktail of melanoma peptides (3 mg) overnight in rotating spinner at 25 RPM for surface adsorption and interaction of peptide with surface modified nanoparticle. Additionally, in some preferred reactions Poly-I:C, IL-12, GM-CSF, IL4, IL-15 and more have been added as secondary adjuvant molecules. For animal vaccine study 3 doses of vaccine equivalent to 20 mg/kg CA3 adjuvant and 30 mg/kg of peptide were injected subcutaneously in mice models. FIG. 17 shows SEM image (1701) of cancer vaccine prepared.

### Example 18: In Vitro Activation of Dendritic Cells by the Nanoparticle Adjuvant Treated Cells

Dendritic cell activation studies showing enhanced CD86 expression, which is a bio-marker for DC activation, M(5)A(3)-1, M(5)A(3)-2, and M(5)A(3)-3 nanoparticle-adjuvant treated cells.

For activation study, 1×10 5 Jaws II cells will be seeded in a 12 well plate and incubate at 37° C. for 24 hours. After incubation, treat the cells with particles as per the concentrations. After treatment, the incubate the cells at 37° C. for 24 hr. After the completion of treatment period, the expression level of immune markers such as CD86 was analysed by FACS.

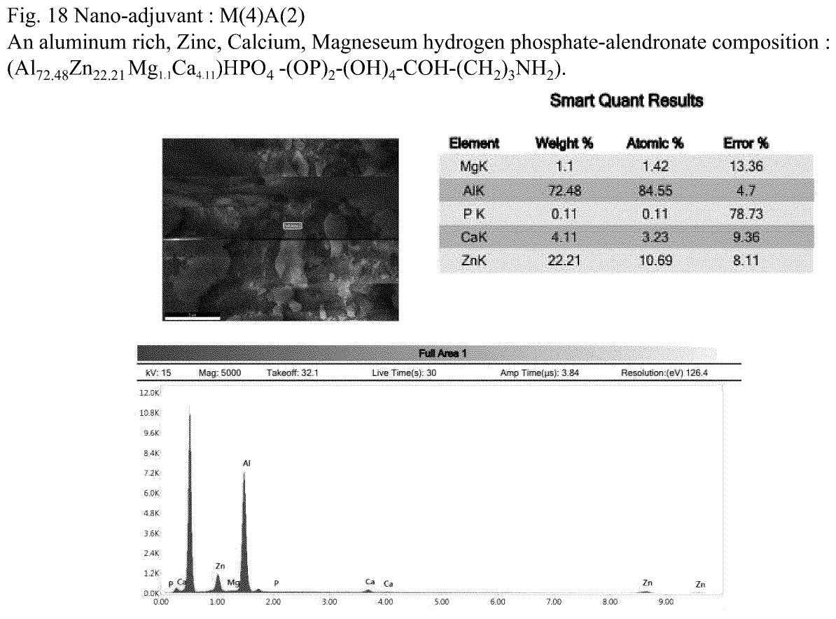

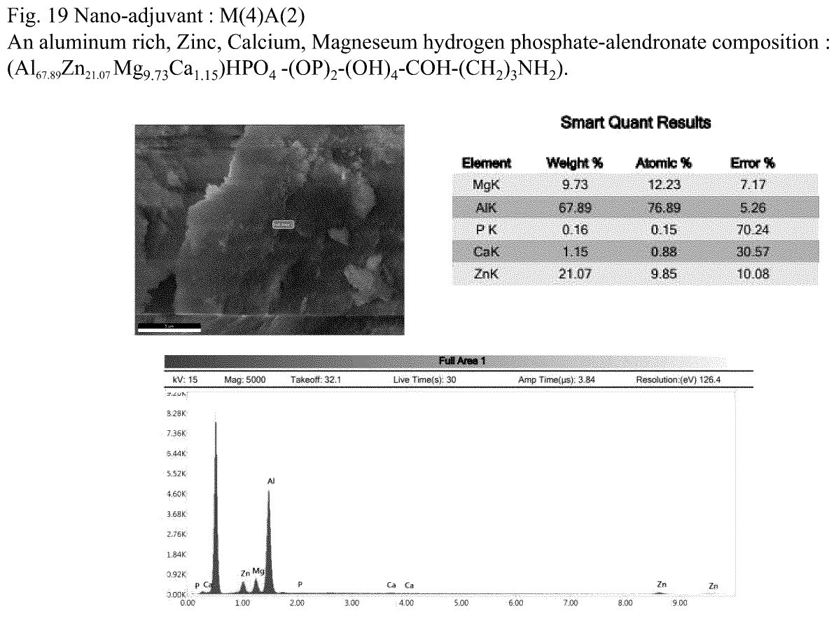

Results as depicted in FIGS. 18 & 19 showed enhanced CD86 expression by two different type of nano-adjuvant particles: M(5)A(3)1-3 (1801)& M(3)A(3)-1 (1802). Scatter plot as well as mean fluorescence intensity shows enhanced expression of CD86 co-stimulatory marker in nano-adjuvant treated cells (1803, 1804, 1805, 1901).

### Example 19: In Vitro Activation of Macrophage Toll Like Receptor by the Nanoparticle Adjuvant Treated Cells

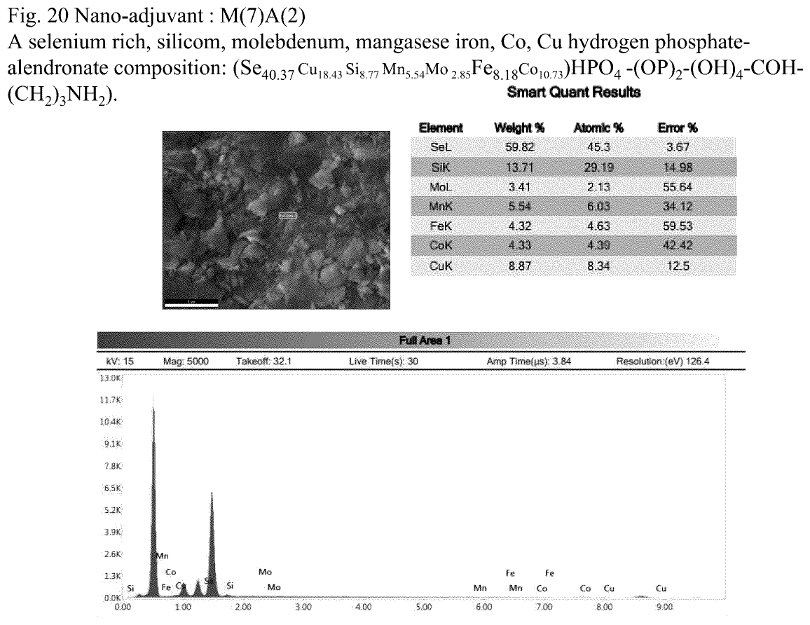

Macrophage Toll like receptor activation by representative M(5)A(3)-1 nanoparticle-adjuvant was done as depicted in FIG. 20. Foe the study, 1×10 5 RAW macrophage were seeded in a 12 well plate and incubate at 37° C. for 24 hours. After incubation, treat the cells with particles as per the concentrations. After treatment, the incubate the cells at 37° C. for 24 hr. After the completion of treatment period, the expression level of TLRs were studied by RT-PCR for fold change in gene expression. The data shows that the inventive nanoparticles, represented as M(n)A(m) sample showed combinatorial activation of TLR-3, TLR-4, TLR-6 and TLR-8 which is very unique compared to the standard adjuvants such as Alum or Poly-IC, LPS or resiquimod which activates maximum 01 or 02 TLRs together. TLR activation is a critical requirement for adjuvants for innate immune activation. FIG. 20 depicts the TLR activation study for TLR3 (2001), TLR 4 (2002), TLR6 (2003) and TLR 8 (2004)

### Example 20: In Vitro Proliferation of Human Mononuclear Cells by the Nanoparticle Adjuvant Treated Cells

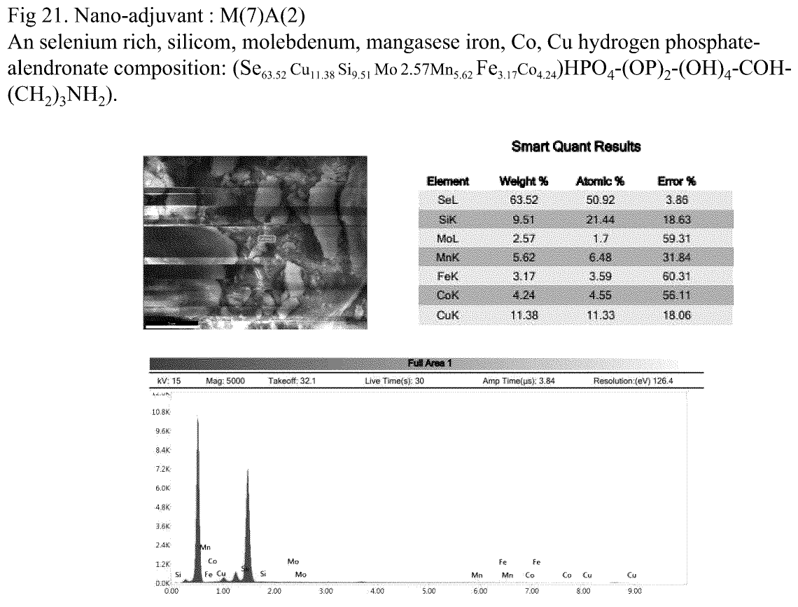

The ability of M(5)A(1)-1, M(5)A(1)-2, and M(5)A(1)-3 nanoparticle-adjuvants to enhance the proliferation of human mononuclear cells within 24-48 Hours using alamar blue assay was studied. PBMCs were isolated from human blood using Ficoll-Hypaque density gradient centrifugation method. Seeded 3×10⁴ cells in 96 well plate and incubated at 37° C. for 24 hours. After incubation, the cells were treated with different concentrations of particles and incubated for 24 hr at 37° C. After 24 hours of treatment, Alamar blue was added to each well (Performed under dark condition), and incubated the plate for overnight at 37° C. After incubation with viable cells, the alamar reagent changes colour from blue to red. The fluorescence intensity was measured at 570 and 600 nm using microplate reader. Results are depicted in FIG. 21, demonstrating that the M(5)A(1)-1 (2101), M(5)A(1)-2 (2102), and M(5)A(1)-3 (2003) nano-adjuvant particle has enormous capacity to enhance the proliferation of T cells up to 03 fold within 24-48 hrs. This is a critical data because T cell-proliferation is a critical requirement of anti-cancer immune response.

### Example 21: In Vivo Analysis of Nanoparticle Adjuvant Based Human Papilloma Virus Vaccine Enhancement of B Plasma Cells and T Follicular Cell Response in Vaccinated Mice

Flowcytometry assay showing the ability of inventive nanoparticle adjuvant based Human papilloma virus vaccine to enhance the B plasma cells and T follicular cell response in vaccinated mice (A) BM: Bone marrow, (B) LN: Lymph node). In vaccinated animals, after two doses within a gap of 14 days, animals were euthanised and organs were harvested. A single cell suspension of spleen/Lymph node/Tumour was made and the cells were pelleted down at 500×g and washed twice with cold PBS. RBC cells were lysed using RBC lysis buffer and further a PBS wash was given. 10⁶ cells are counted and re-suspended in suitable volume of staining buffer.

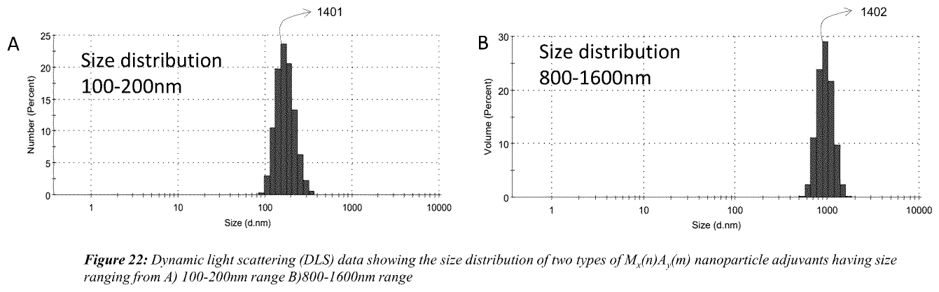

For T cell analysis, the pan immune marker CD45 was used to separate immune cells from which D3+, CD4+ and CD8+ cells were gated and activation marker CD69 was also gated. For B cell analysis, from CD45+ cells, CD19+/B220+ cells were analysed for B mature cells showing IgD-CD38+/−CD27+ expression. For Tfh cell staining, CD45+ cells were further gated for CD3+CD4+, CXCR5+ and activation markers ICOS, PD1 for flow cytometry analysis. FIG. 22 shows the enhanced plasma B cells, 2201, and THF cell response, 2202, 2203 in a representative nanoparticle M(5)A(1)-3 vaccinated animals.

### Example 22: In Vivo Antibody Generation and Enhanced T Cell Response Against SARS COV2 Virus Using M(5)A(1)-RBD1 Vaccination

Evaluation of Antibody titre in serum samples of experimental animals (C57BL/6J mice) treated with RBD sequence of SARS-COV2 S peptide-based nanoparticle adjuvant vaccine in comparison with a standard Alum vaccine was assessed.

Study Description: The inventors vaccinated the animals using the said nano-adjuvant (M(5)A(1)-1) complexed with SARS-Cov2-RBD peptide antigen sequence, prepared as per the method described in example 3 and administered in two doses on Day 0 and Day 21. A standard adjuvant, ALUM based vaccine of same RBD sequence and concentration, and untreated animal serum were taken as control. Each vaccine contains 5 ug of peptide and 10 mg/kg of nanoparticles.

Further, the serum samples were collected prior to each intramuscular injection (I.M.) of vaccine doses and final serum sample was collected on day 28. Samples were analysed for anti-RBD specific IgG antibody production at serum dilutions 1/100, 1/1000, 1/5000, 1/10000 using ELISA readout.

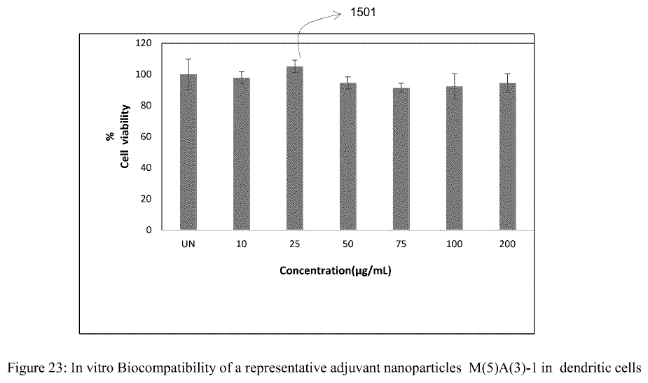

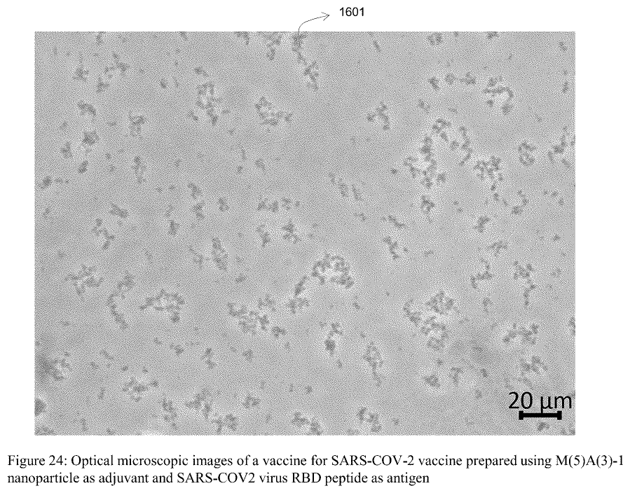

FIG. 23 shows antibody titre for unvaccinated animal serum (control), Alum based Spike-RBD peptide vaccine (S+Alum) and the inventive adjuvant nanoparticle M (5)A(1)-1 based Spike-RBD peptide vaccine (S+CA3), 2302. As shown in the FIG. 23, it was observed that the serum from animal vaccinated with M(5)A(1)-1 adjuvant nanoparticle (2301) showed highest concentration of antibody against the SARS-Cov2 RBD peptide, compared to alum-based vaccine, 2302. Further, FIG. 24 shows virus neutralization assay indicating that the inventive nanoparticle has >97% neutralization antibody, 2403, 2404, 2405, compared to 16.5% of peptide alone (2401, 2402). This clearly shows that the inventive nanoparticle has superior adjuvant properties than commercial adjuvant vaccines.

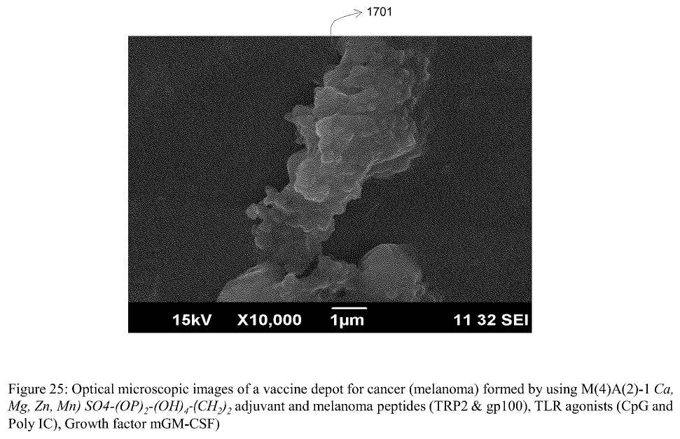

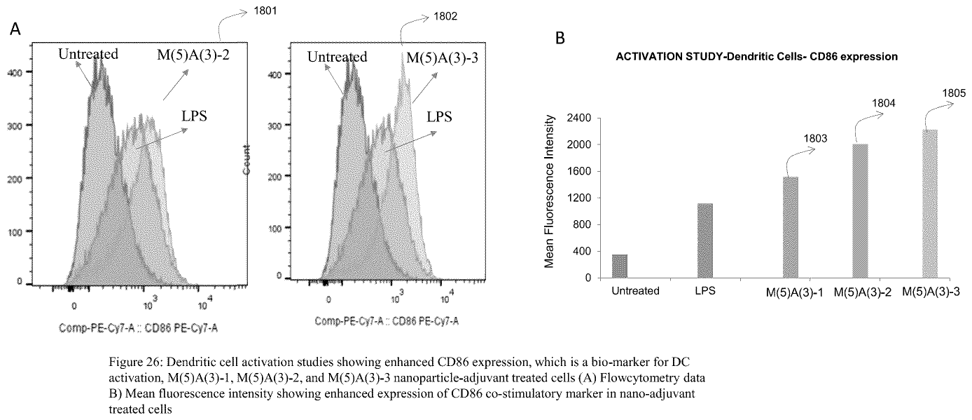

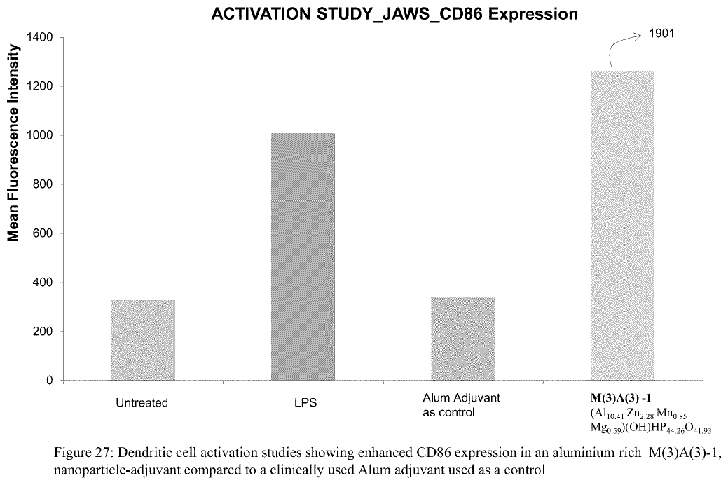

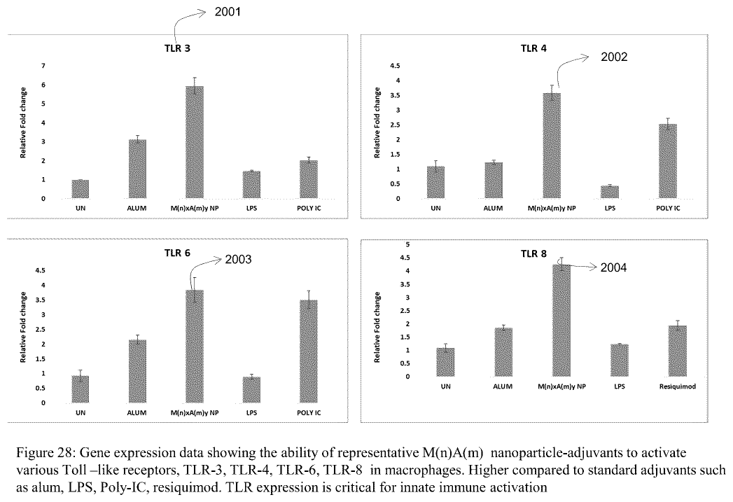

In addition to better antibody data, the ELISPOT data (FIG. 25) showed the ability of (M(n)ₓA(m)_{y}) nanoparticle-adjuvants to enhance the interferon-gamma expressing capacity of T cells in a vaccinated animal, (2502) when re-challenged with same antigen compared to a standard adjuvant alum-based vaccine 2501). Further, it was demonstrated that these nanoparticles are capable of enhancing the antibody as well as T cell response when they are mixed with other commercial vaccines such as Abbot flu vaccine and Gardasil IHPV vaccines as depicted in results shown in FIGS. 26, 27 and 28. In case of Abbot vaccines, compared to current clinically used flu vaccine of Abbot (2601, 2602), our inventive nano-adjuvant blended abbot vaccine (2603, 2604) showed higher interferon expressing immune cells. Similarly in case of Gardasil vaccine against IHPV infection, compared to Gardasil alone (2803, 2804) or Gardasil plus alum adjuvant (2802), nano-adjuvant blended Gardasil vaccine (2801, 2805) showed enhanced IFN gamma response in ELIPOT study.

### Example 23: In Vivo Anti-Tumour Immune Response by Nanoparticles-Based Cancer Vaccines

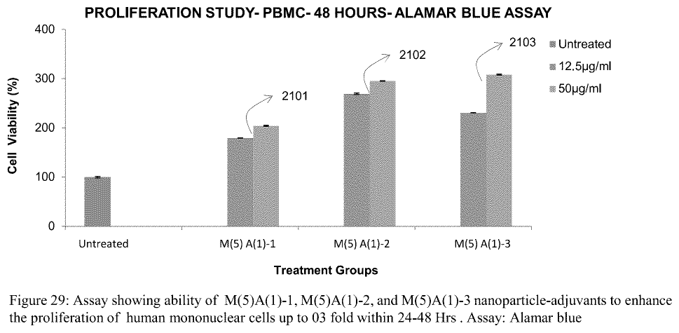

The effect of cancer-vaccination with (M(n)ₓA(m)_{y}) nanoparticle-adjuvants was studied in melanoma. FIG. 29 shows tumour volume changes with time in untreated, 2901 versus (M(n)ₓA(m)_{y}) nanoparticle, 2902, based TRP2-GP100 vaccine treated animal. Corresponding optical images of melanoma tumour in treated and untreated animal showed significant tumour reduction in vaccinated group.

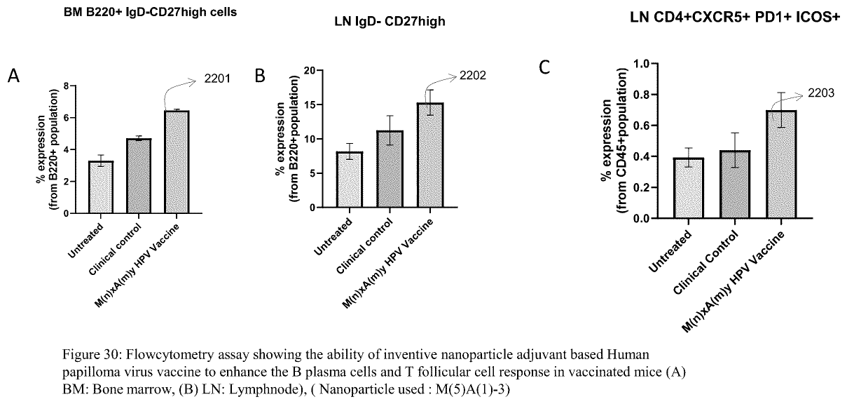

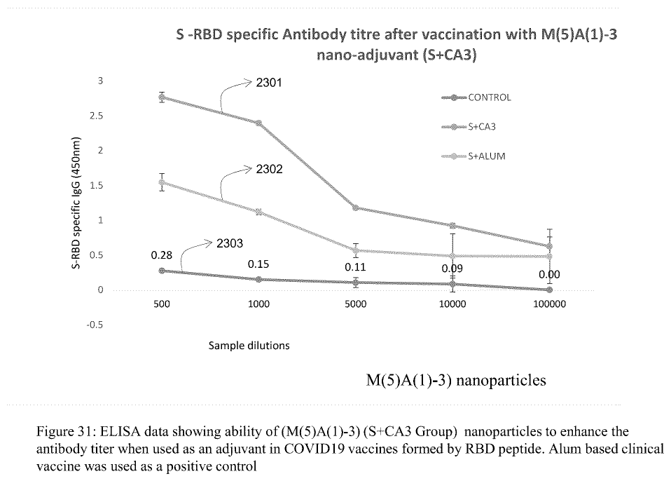

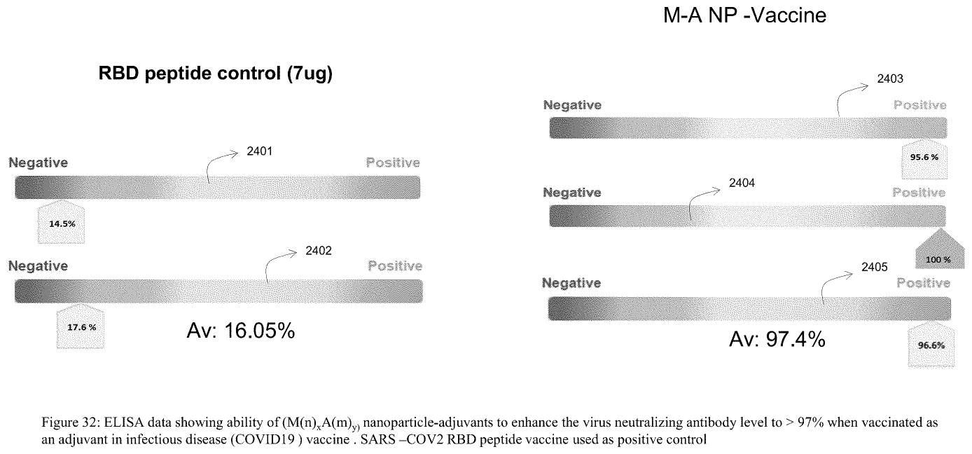

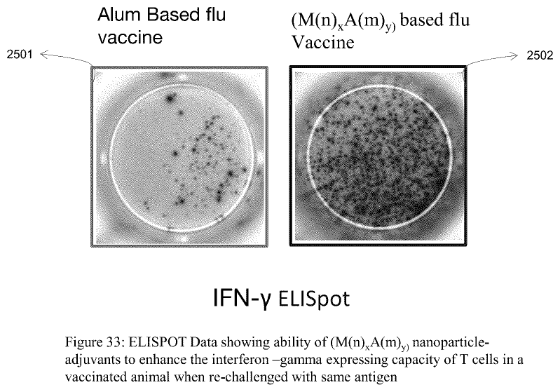

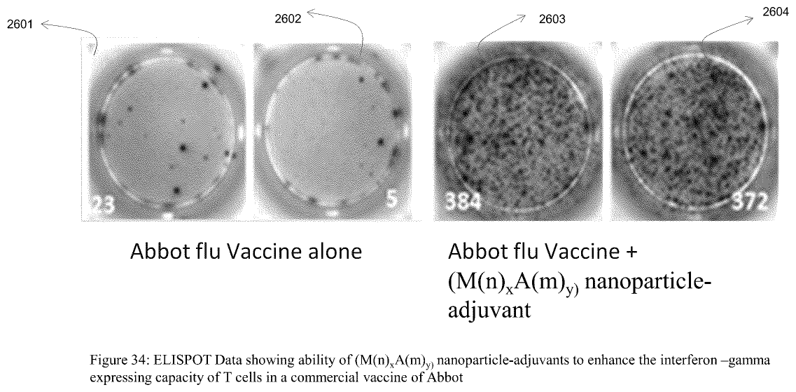

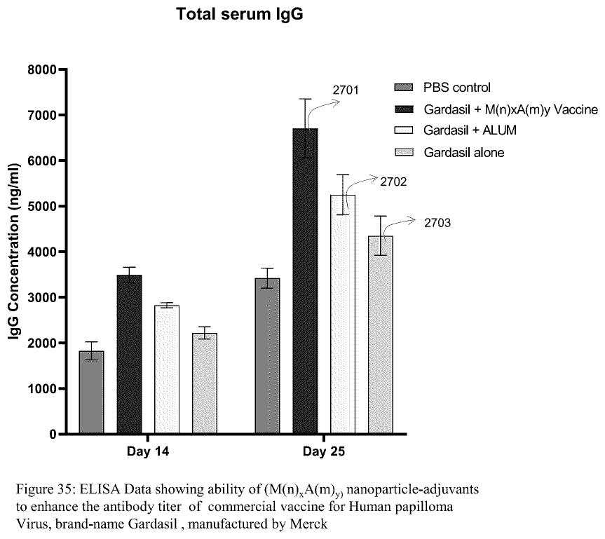

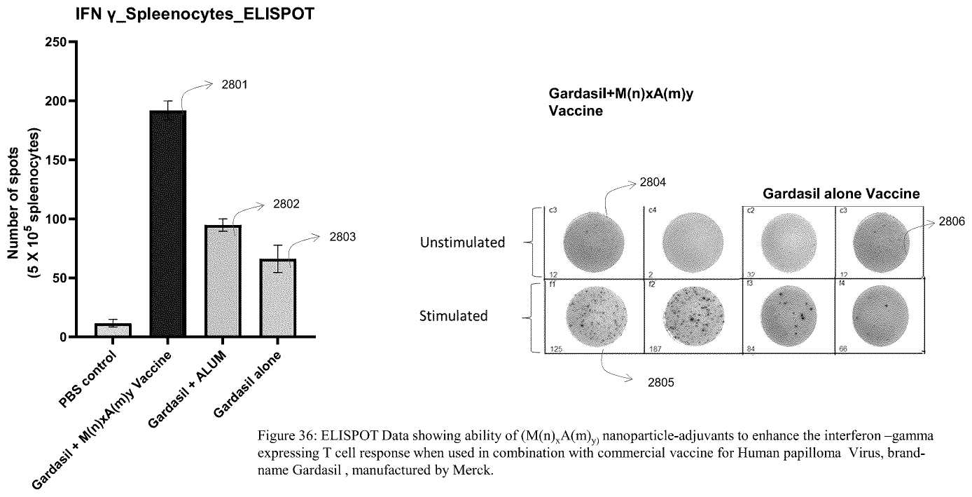

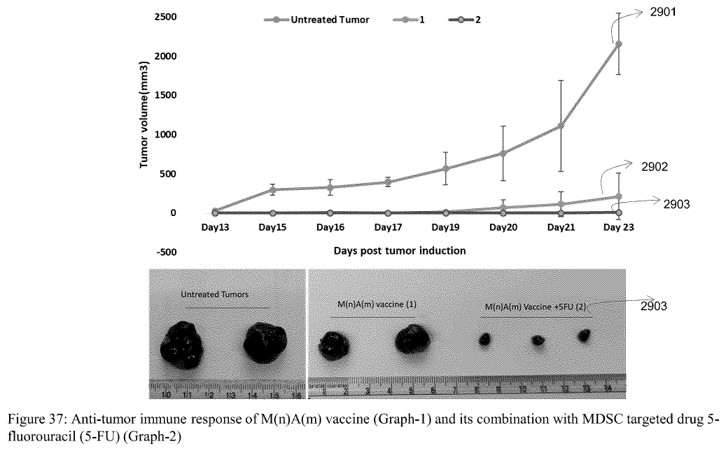

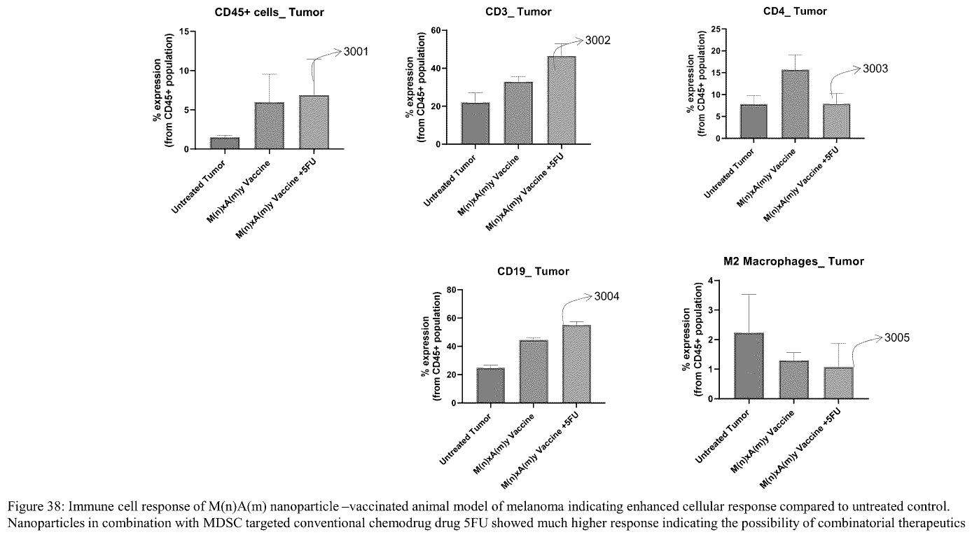

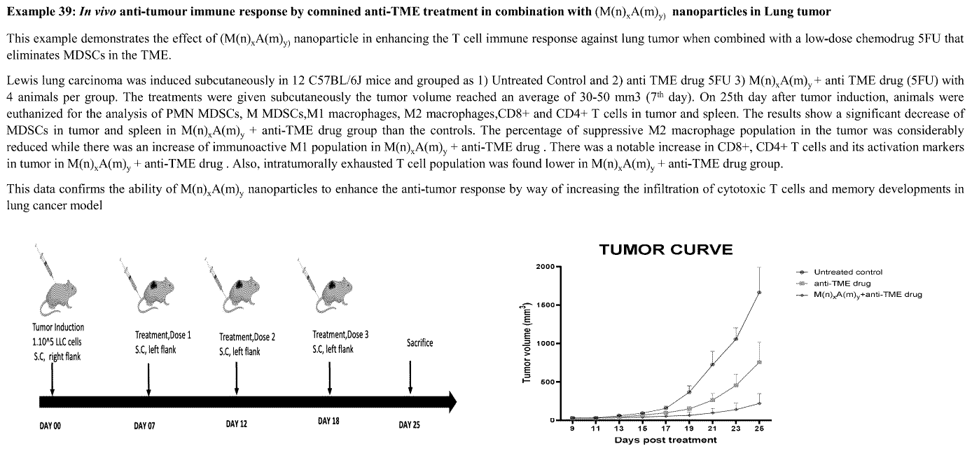

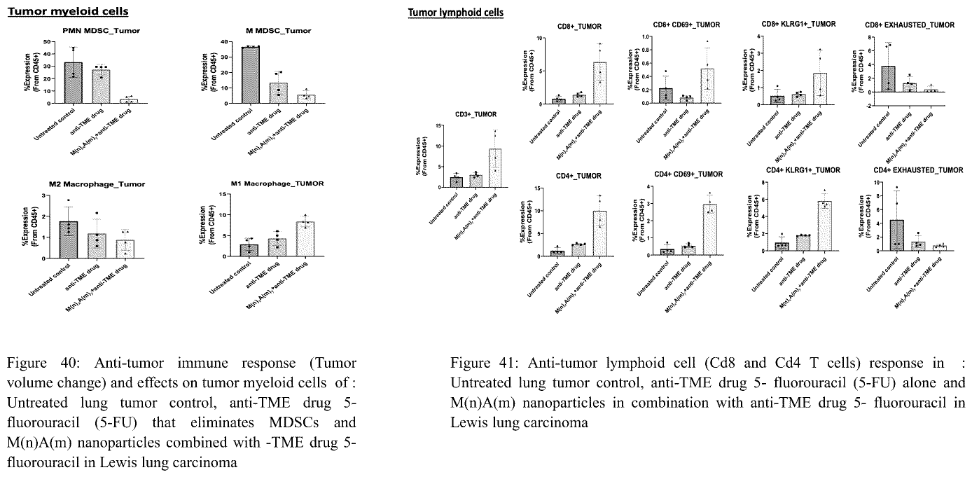

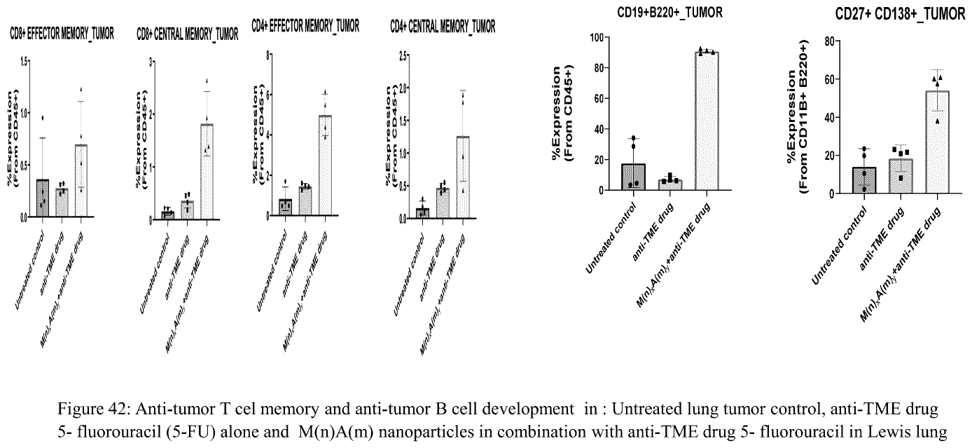

Nanoparticle plus MDSC targeted drug 5FU showed much better control on tumour growth. FIG. 30 showed immune cell response of M(n)A(m) nanoparticle-vaccinated animal model of melanoma indicating enhanced cellular response for CD45, 3001, CD3, 3002, CD4, 3003, CD19, 3004, M2 macrophages, 3005 compared to untreated control. Nanoparticles in combination with MDSC targeted conventional chemo drug 5FU showed much higher response indicating the possibility of combinatorial therapeutics.

### Example 24: In Vivo Anti-Tumour Immune Response by Combined Anti-TME Treatment in Combination with (M(n)ₓA(m)_{y}) Nanoparticles in Lung Tumor

This example demonstrates the effect of (M(n)ₓA(m)_{y}) nanoparticle in enhancing the T cell immune response against lung tumor when combined with a low-dose chemodrug 5FU that eliminates MDSCs in the TME.

Lewis lung carcinoma was induced subcutaneously in 12 C57BL/6J mice and grouped as 1) Untreated Control and 2) anti TME drug 5FU 3) M(n)ₓA(m)_{y}+anti TME drug (5FU) with 4 animals per group. The treatments were given subcutaneously the tumor volume reached an average of 30-50 mm3 (7^{th} day). On 25th day after tumor induction, animals were euthanized for the analysis of PMN MDSCs, M MDSCs, M1 macrophages, M2 macrophages, CD8+ and CD4+ T cells in tumor and spleen. The results show a significant decrease of MDSCs in tumor and spleen in M(n)ₓA(m)_{y}+anti-TME drug group than the controls. The percentage of suppressive M2 macrophage population in the tumor was considerably reduced while there was an increase of immunoactive M1 population in M(n)ₓA(m)_{y}+anti-TME drug. There was a notable increase in CD8+, CD4+ T cells and its activation markers in tumor in M(n)ₓA(m)_{y}+anti-TME drug. Also, intratumorally exhausted T cell population was found lower in M(n)ₓA(m)_{y}+anti-TME drug group.

This data confirms the ability of M(n)ₓA(m)_{y} nanoparticles to enhance the anti-tumor response by way of increasing the infiltration of cytotoxic T cells and memory developments in lung cancer model.

The foregoing description of the specific embodiments fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Throughout this specification, the word “comprises”, or variations such as “comprises” or “comprising” wherever used, will be understood to imply the inclusion of a stated element, integer or step, or group of elements, integers or steps, but not the exclusion of any other element, integer or step, or group of elements, integers or steps. Similarly, terms such as “include” or “have” or “contain” and all their variations are inclusive and will be understood to imply the inclusion of a stated element, integer or step, or group of elements, integers or steps, but not the exclusion of any other element, integer or step, or group of elements, integers or steps.

As used herein, the term ‘comprising’ when placed before the recitation of steps in a method means that the method encompasses one or more steps that are additional to those expressly recited, and that the additional one or more steps may be performed before, between, and/or after the recited steps. For example, a method comprising steps a, b, and c encompasses a method of steps a, b, x, and c, a method of steps a, b, c, and x, as well as a method of steps x, a, b, and c. Furthermore, the term “comprising” when placed before the recitation of steps in a method does not (although it may) require sequential performance of the listed steps, unless the content clearly dictates otherwise. For example, a method comprising steps a, b, and c encompasses, for example, a method of performing steps in the order of steps a, c, and b, the order of steps c, b, and a, and the order of steps c, a, and b, etc. The terms “about” or “approximately” are used herein to mean approximately, in the region of, roughly, or around. When the term “about” is used in conjunction with a numerical value/range, it modifies that value/range by extending the boundaries above and below the numerical value(s) set forth. In general, the term “about” is used herein to modify a numerical value(s) or a measurable value(s) such as a parameter, an amount, a temporal duration, and the like, above and below the stated value(s) by a variance of +/−20% or less, +/−10% or less, +/−5% or less, +/−1% or less, and +/−0.1% or less of and from the specified value, insofar such variations are appropriate to perform in the disclosed invention, and achieves the desired results and/or advantages as disclosed in the present disclosure. It is to be understood that the value to which the modifier “about” or “approximately” refers is itself also specifically, and preferably, disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The suffix ‘(s)’ at the end of any term in the present disclosure envisages in scope both the singular and plural forms of said term.

Numerical ranges stated in the form ‘from x to y’ include the values mentioned and those values that lie within the range of the respective measurement accuracy as known to the skilled person. If several preferred numerical ranges are stated in this form, of course, all the ranges formed by a combination of the different end points are also included.

Throughout this specification, the term ‘a combination thereof’, ‘combinations thereof’ or ‘any combination thereof’ or ‘any combinations thereof’ are used interchangeably and are intended to have the same meaning, as regularly known in the field of patent disclosures.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

All references, articles, publications, general disclosures etc. cited herein are incorporated by reference in their entireties for all purposes. However, mention of any reference, article, publication etc. cited herein is not, and should not be taken as, an acknowledgment or any form of suggestion that they constitute valid prior art or form part of the common general knowledge in any country in the world.

What is claimed is:

1. A non-stoichiometric multi-element adjuvant (NSMA) comprising a combination of cations and anions as represented by a general formula: 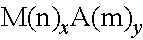
where ‘M’ represents cation elements,
‘n’ represents the number of cations used in each compound which is at least 2,
‘A’ represents the type of anion groups and ‘m’ represents the numbers of anion groups which is at least 1,
‘x’ and ‘y’ represents weight percentage of each cation or anion, respectively, varying from 0.01 to 99%.

2. The adjuvant as claimed in claim 1, wherein the non-stoichiometric combination of at least two cations (M), is selected from the group consisting of Zn, Al, Ca, Sr, Ba, Mn, Mo, Si, Cu, Ni, Sn, Co, Fe, Cr, Se, Na, K, and/or Mg.

3. The adjuvant as claimed in claim 1, wherein the non-stoichiometric combination of at least one anion (A), is selected from the group consisting of PO₄, OH, HPO₄, H₂PO₄, OHPO₄, (OH)H—PO₄, (OH)H₂PO₄, [SiO₂₊ₙ]2n-, SO₄, phosphonates, bisphosphonate (PO(OH)₂)₂-R1-R2, (SO₄), (O—OH), (SO₄)(PO₄), (SO₄)(OH), (SO4)(OH)(PO₄), (PO₄)(OH), (SiO4)(OH), (OH)(PO₄)(SiO₄), PO₃—R, (PO₃—R), (PO4)(PO₃—R), (PO₂—R)(OH), (OH)₂PO)₂—R, or combination thereof,
wherein R can be selected from ═CH₃, C₂H₃, C₆H₅, (CH2)₂COOH;
R1=halogen, H or OH, and
R2=CH₃, halogen, (CH₂)₂NH₂, (CH₂)₅NH₂, (CH₂)₂N(CH₃)₂, (CH₂)₃NH₂, (CH₂)₂N(CH₃)(CH₂)₄(CH₃), CH₂)N(CH₂)NCH, (CH₂)N(C(CH)₄, (CH)S(C₆H₄)Cl.

4. The adjuvant as claimed in claim 1, wherein one of the anion A is selected from O₂P₂—(OH)₄—C—Cl2, O₂P₂—(OH)₄—COH—CH3, O₂—P₂—(OH)₄—COH—(CH₂)₂NH₂, O₂P₂—(OH)₄—COH—(CH₂)₂N(CH₃)₂, O₂—P₂—(OH)₄—COH—(CH₂)₃NH₂, O₂P₂—(OH)₄—COH—(CH₂)₂N(CH₃)(CH₂)₄(CH₃), O₂P₂—(OH)₄—COH—(CH₂)N(CH₂)NCH, O₂P₂—(OH)₄—COH—(CH₂)N(C(CH)₄, and/or O₂P₂—(OH)₄—COH—(CH)S(C₆H₄)Cl

5. The adjuvant as claimed in claim 1, wherein the compound MA is formed by a combination of cations; M and anions; A and selected from any of the following compounds: (M)(PO₄), (M)(SO₄), M(O—OH), (M)(SO₄)(PO4), (M)(SO₄)(OH), (M)(SO4)(OH)(PO₄), (M)(PO₄)(OH), (M)(HPO₄)(OH), (M)(H₂PO₄)(OH), (M)(SiO4)(OH), (M)(OH)(PO₄)(SiO₄), (M)PO₃—R, (M)(PO₃—R), (M)(PO4)(PO₃—R), (M)(PO₂—R)(OH), (M)((OH)₂PO)₂—R, with R═CH₃, C₂H₃, C₆H₅, (CH₂)₂COOH, (M)-O₂P₂—(OH)₄—C—Cl2, (M)-O₂P₂—(OH)₄—COH—CH3, (M)-O₂—P₂—(OH)₄—COH—(CH₂)₂NH₂, (M)-O₂P₂—(OH)₄—COH—(CH₂)₂N(CH₃)₂, (M)-O₂—P₂—(OH)₄—COH—(CH₂)₃NH₂, (M)-O₂P₂—(OH)₄—COH—(CH₂)₂N(CH₃)(CH₂)₄(CH₃), (M)-O₂P₂—(OH)₄—COH—(CH₂)N(CH₂)NCH, (M)-O₂P₂—(OH)₄—COH—(CH₂)N(C(CH)₄, and/or (M)-O₂P₂—(OH)₄—COH—(CH)S(C₆H₄)Cl, where M is selected from at least two cations from the group of: Zn, Al, Ca, Sr, Ba, Mn, Mo, Si, Cu, Ni, Sn, Co, Fe, Cr, Se, Na, K, and/or Mg.

6. The adjuvant as claimed in claim 1, wherein the adjuvant is a multi-cation adjuvant selected from
a. bi-cation adjuvant of formula: M(2)A(m),
b. tri-cation adjuvant of formula: M(3)A(m),
c. tetra-cation adjuvant of formula: M(4)A(m),
d. penta-cation adjuvant of formula: M(5)A(m),
e. hexa-cation adjuvant of formula: M(6)A(m),
f. hepta-cation adjuvant of formula: M(7)A(m),
g. octa-cation adjuvant of formula: M(8)A(m),
h. nona-cation adjuvant of formula: M(9)A(m),
i. deca-cation adjuvant of formula: M(10)A(m), and
j. undeca-cation adjuvant of formula: M(11)A(m).

7. The adjuvant as claimed in claim 6, wherein the non-stochiometric adjuvants adjuvant compounds comprising the molecular formula represented by:

| | |
|---|---|
| (Zn, Al, Ca, Mn, Mg)(PO4)₂ | (Zn, Al, Ca, Mn, Mg)₂(PO₄)₃(OH)₃ |
| (Zn, Al, Ca, Mn, Mg)(HPO4)₂ | (Zn, Al, Ca, Mn, Mg)₂(HPO₄)₃(OH)₃ |
| (Zn, Al, Ca, Mn, Mg)(H₂PO4)₂ | (Zn, Al, Ca, Mn, Mg)₂(H₂PO₄)₃(OH)₃ |
| (Zn, Al, Ca, Mn, Mg)(HPO4) | (Zn, Al, Ca, Mn, Mg)₃(PO4)₂ |
| (Zn, Al, Ca, Mn, Mg)((HPO₄)(H₂O)₂ | (Zn, Al, Ca, Mn, Mg)₃(HPO4)₂ |
| (Zn, Al, Ca, Mn, Mg)(PO₄)₂(OH)₃ | (Zn, Al, Ca, Mn, Mg)₃(H₂PO4)₂ |
| (Zn, Al, Ca, Mg)(HPO₄)₂(OH)₃ | (Zn, Al, Ca, Mn, Mg)₃(PO4)₂(OH)₃ |
| (Zn, Al, Ca, Mn, Mg)(H₂PO₄)₂(OH)₃ | (Zn, Al, Ca, Mn, Mg)₃(HPO4)₂(OH)₃ |
| (Zn, Al, Ca, Mg)₂(PO4)₃ | (Zn, Al, Ca, Mn, Mg)₃(H₂PO4)₂(OH)₃ |
| (Zn, Al, Ca, Mn, Mg)₂(HPO4)₃ | (Zn, Al, Ca, Mn, Mg)₃(PO₄)(OH)₂ |
| (Zn, Al, Ca, Mn, Mg)₂(H₂PO4)₃ | (Zn, Al, Ca, Mn, Mg)₃(HPO₄)(OH)₂ |
| (Zn, Al, Ca, Mn, Mg)₃(H₂PO₄)(OH)₂ | (Zn, Al, Ca, Mn, Mg)₄(PO₄)₃ |
| (Zn, Al, Ca, Mn, Mg)₄(HPO₄)₃ | (Zn, Al, Ca, Mn, Mg)₄(H₂PO₄)₃ |
| (Zn, Al, Ca, Mn, Mg)₄(PO₄)₃(OH)₄ | (Zn, Al, Ca, Mn, Mg)₄(HPO₄)₃(OH)₄ |
| (Zn, Al, Ca, Mn, Mg)₄(H₂PO₄)₃(OH)₄ | (Zn, Al, Ca, Mn, Mg)₆(PO₄)₄(OH)₈ |
| (Zn, Al, Ca, Mn, Mg)₆(HPO₄)₄(OH)₈ | (Zn, Al, Ca, Mn, Mg)₆(H₂PO₄)₄(OH)₈ |
| (Zn, Al, Ca, Mg)₈H₂(PO₄)₆ | (Zn, Al, Ca, Mn, Mg)(PO₄)₆PO₃OH |
| (Zn, Al, Ca, Mn, Mg)₂P₂O₇ | (Zn, Al, Ca, Mn, Mg)₅(P₃O₁₀)₂ |
| (Zn, Al, Ca, Mn, Mg)₅(PO₄)₃(OH) | (Zn, Al, Ca, Mg)₁₀(PO₄)₆(OH, F, Cl, Br, I)₂ |
| (Zn, Al, Ca, Mn, Mg)₄(PO₄)₂O | (Zn, Al, Ca, Mg)₃(H₂PO₄)(OH)₂—C—R1—R2 |
| | where R1 = H, OH, Cl and R2 = CH₃, Cl, |
| | (CH2)₂NH2, (CH₂)₅NH₂, (CH₂)₂N(CH₃)₂, |
| | (CH₂)₃NH₂, (CH₂)₂N(CH₃)(CH₂)4CH₃, |
| | C₇H₁₀NNaP2O₇, C₅H₁₀N₂PO₇, or C₅H₁₂N₂P2O₈ |
| (Mn, Al, Ca, Zn, Mg)((OH)₂PO)₂—C—H—R2 | (Al, Ca, Zn, Mg)((OH)₂PO)₂—C—OH—R2 |
| R2 = CH3, Cl, (CH2)₂NH2, (CH₂)₅NH₂, | R2 = CH₃, Cl, (CH2)₂NH2, (CH₂)₅NH₂, |
| (CH₂)₂N(CH₃)₂, (CH₂)₃NH₂, | (CH₂)₂N(CH₃)₂, (CH₂)₃NH₂, |
| (CH₂)₂N(CH₃)(CH₂)4CH₃, | (CH₂)₂N(CH₃)(CH₂)4CH₃, |
| C7H10NNaP2O7, C5H10N2PO7, or | C7H10NNaP2O7, C5H10N2PO7, or |
| C5H12N2P2O8 | C5H12N2P2O8 |
| (Al, Ca, Zn, Mg)((OH)₂PO)₂—C—Cl2—R2 | (Al, Ca, Zn, Mg)H(PO)₄—C—H—R2 |
| where | R2 = CH3, Cl, (CH2)₂NH2, (CH₂)₅NH₂, |
| R2 = CH3, Cl, (CH2)₂NH2, (CH₂)₅NH₂, | (CH₂)₂N(CH₃)₂, (CH₂)₃NH₂, |
| (CH₂)₂N(CH₃)₂, (CH₂)₃NH₂, | (CH₂)₂N(CH₃)(CH₂)4CH₃, |
| (CH₂)₂N(CH₃)(CH₂)4CH₃, | C7H10NNaP2O7, C5H10N2PO7, or |
| C7H10NNaP2O7, C5H10N2PO7, or | C5H12N2P2O8 |
| C5H12N2P2O8 | |
| (Al, Ca, Zn, Mg)(H)₂PO)₄ | (Al, Ca, Zn, Mg)(H)₂PO)₄—C—H—R2 |
| | R2 = CH3, Cl, (CH2)₂NH2, (CH₂)₅NH₂, |
| | (CH₂)₂N(CH₃)₂, (CH₂)₃NH₂, |
| | (CH₂)₂N(CH₃)(CH₂)4CH₃, |
| | C7H10NNaP2O7, C5H10N2PO7, or |
| | C5H12N2P2O8 |
| Al_{76.41}Zn_{17.83}Mg_{4.72}, Ca_{0.94})HPO₄—(OP)₂— | Al_{76.09}Zn_{14.56}Mg_{6.07}, Ca_{3.2})HPO₄—(OP)₂— |
| (OH)₄—COH—(CH₂)₃NH₂). | (OH)₄—COH—(CH₂)₃NH₂). |
| Al_{89.35}Zn_{0.36}Mg_{8.46}, Ca_{1.58})HPO₄—(OP)₂— | (Al_{83.9}Zn_{4.89}Mg_{7.59}Ca_{3.42})HPO₄—(OP)₂— |
| (OH)₄—COH—(CH₂)₃NH₂). | (OH)₄—COH—(CH₂)₃NH₂). |
| (Al_{72.48}Zn_{22.21}Mg_{1.1}Ca_{4.11})HPO₄—(OP)₂— | Al_{67.89}Zn_{21.07}Mg_{9.73}Ca_{1.15})HPO₄—(OP)₂— |
| (OH)₄—COH—(CH₂)₃NH₂). | (OH)₄—COH—(CH₂)₃NH₂ |
| Se_{40.37}Cu_{18.43}Si_{8.77}Mn_{5.54}Mo_{2.85} | Se_{40.37}Cu_{18.43}Si_{8.77}Mo_{2.85} |
| Fe_{8.18}Co_{10.73})HPO₄—(OP)₂—(OH)₄—COH— | Mn_{10.68}Fe_{8.18}Co_{10.73})HPO₄—(OP)₂—(OH)₄— |
| (CH₂)₃NH₂). | COH—(CH₂)₃NH₂). |
| Se_{63.52}Cu_{11.38}Si_{9.51}Mo2.57Mn_{5.62} | |
| Fe_{3.17}Co_{4.24})HPO₄—(OP)₂—(OH)₄—COH— | |
| (CH₂)₃NH₂) | |

8. The adjuvant as claimed in claim 1, wherein the non-stoichiometric adjuvant is a multi-cation-multi anion adjuvant selected from
a. an adjuvant comprising at least 5 cations linked to at least one anion and is represented by the general formula: M(5)A(1), or
b. an adjuvant comprising at least 5 cations linked to at least two anions and is represented by the general formula: M(5)A(2), or
c. an adjuvant comprising at least 5 cations linked to at least three and is represented by the general formula: M(5)A(3), or
d. an adjuvant comprising at least 4 cations linked to at least 2 anions and is represented by general formula: M(4)A(2), or
e. an adjuvant comprising at least 3 cations linked to at least two anions and is represented by the molecular formula: M(3)A(2), or
f. an adjuvant comprising at least 2 cations linked with two anions and is represented by the general formula: M(2)A(2), or
g. an adjuvant comprising at least 2 cations linked with three anions and is represented by the general formula: M(2)A(3).

9. The adjuvant as claimed in claim 8, wherein the non-stochiometric multi-cation and multi anion adjuvant compounds have the molecular formula represented by:

| | |
|---|---|
| (Ca, Mn, Zn, Al, Mg)ₓ(HPO₄)_{y}, | (Al, Zn, Ca, Mg)ₓ(HPO₄)_{y}, |
| where weight percentage (x) of Zn can vary | where weight percentage (x) of Al |
| from 0.01-99 w %, | concentration range from 60 to 95 wt % |
| Al concentration can vary from 0.01-99 w %, | Zn concentration range from 0.1 to |
| Ca concentration can vary from | 25 wt % |
| 0.01-99 w %, | Ca concentration range from 0.1 to |
| Mn concentration can vary from 0.01 to 99 | 25 wt % |
| w %, | Mg concentration range from 0.1 to |
| Mg concentration can vary from | 25 wt % |
| 0.01-99 w %. | |
| (Zn, Al, Ca, Mn, Mg)3(OH)(HPO4) | (Ca, Zn, Al, Mg)₃(OH)₂(HPO₄)—(OH)₂PO)₂—R1—R2 |
| where Zn varies from 0.01-99 w %, | Where R1 = H, OH, or Cl, |
| Al concentration varies from 0.01-99 w %, | R2 = CH3, Cl, (CH2)₂NH2, (CH₂)₅NH₂, |
| Ca concentration varies from 0.01-99 w %, | (CH₂)₂N(CH₃)₂, (CH₂)₃NH₂, |
| Mn concentration vary from 0.01 to 99 w %, | (CH₂)₂N(CH₃)(CH₂)4CH₃, |
| where Zn concentration varies from 0.01-99 w %, | C7H10NNaP2O7, C5H10N2PO7, |
| Al concentration varies from 0.01-99 w %, | C5H12N2P2O8 and |
| Ca concentration varies from 0.01-99 w %, | where Zn concentration varies from |
| Mg concentration varies from 0.01-99 w % | 0.01-99 w %, |
| | Al concentration varies from 0.01-99 w %, |
| | Ca concentration varies from 0.01-99 w %, |
| | Mn concentration vary from 0.01 to 99w %, |
| | Mg concentration varies from 0.01-99 w % |
| (Al—Mn—Mg)₃(OH)₂(H₂PO₄) | (Al, Zn)₃(OH)₂(HPO₄) |
| where Al concentration varies from | where Al concentration varies from |
| 0.01-99 w %, | 0.01-99 w %, |
| Mn concentration vary from 0.01 to 99 w %, | Zn concentration vary from 0.01 to 99 w %. |
| Mg concentration varies from 0.01-99 w % , | |
| (Al, ZnMg)₃(OH)₂(H₂PO₄)—(OH)₂(PO)₂—R1—R2 | |
| where R1 = H, OH, or Cl and | |
| R2 = CH3, Cl, (CH2)₂NH2, (CH₂)₅NH₂, | |
| (CH₂)₂N(CH₃)₂, (CH₂)₃NH₂, | |
| (CH₂)₂N(CH₃)(CH₂)4CH₃, C₇H10NNaP2O7, | |
| C5H10N2PO7, C5H12N2P2O8 | |
| where Al concentration varies from | |
| 0.01-99 w %, | |
| Zn concentration varies from 0.01 to 99 w %, | |
| Mg concentration varies from 0.01-99 w %. | |

10. The adjuvant as claimed in claim 1, wherein the non-stoichiometric multi-element adjuvant (NSMA) as shown in Formula I: 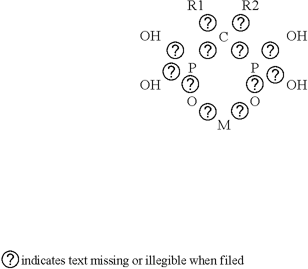
wherein (M) is at least two or more metal ions linked with bisphosphonates having the formula: M-(PO(OH)₂)₂-R1-R2,
R1=H, OH or CH₃,
R2=CH₃, Cl, (CH₂)₂NH₂, (CH₂)₅NH₂, (CH₂)₂N(CH₃)₂, (CH₂)₃NH₂, (CH₂)₂N(CH₃)(CH₂)₄(CH₃), CH₂)N(CH₂)NCH, (CH₂)N(C(CH)₄, (CH)S(C₆H₄)Cl, and
wherein M is a combination of at least two or more cations selected from the group consisting of Mn, Zn, Ca, Ba, Sr, Al, Mg, Se, Cu, Fe, Mo, Cr, Si, Ba, Sr, Sn, K, Na or combinations thereof.

11. The adjuvant as claimed in claim 1, wherein the non-stoichiometric nano-micro sized adjuvant is in the form of spherical particles, needles, rods, flower like structures, petals, star-like structures, prismatic structures, porous structures, and irregular self-assembled composite or core-shell structures with other materials such as proteins, peptides, RNA, DNA, carbohydrates, polymers, lipids, liposomes, lipo-proteins, sugars etc. for the purpose of immuno-activation, vaccination, and stand-alone therapeutics.

12. The adjuvant as claimed in claim 1, wherein the non-stoichiometric adjuvant wherein the size of the adjuvant compound ranges from 1 nm to 10,000 μm, specifically 1-100 nm, 100-500 nm, 500-1000 nm, 1-10 μm, 10-50 μm, 50-100 μm, 100-500 μm, 500-1000 μm, 1000-2000 μm, 2000-3000 μm, 3000-4000 μm, 4000-5000 μm, 5000-6000 μm, 6000-7000 μm, 7000-8000 μm, 8000-9000 μm, 9000-10000 μm.

13. The adjuvant as claimed in claim 1, wherein the non-stoichiometric adjuvant is optionally doped with other elements for creating specific properties such as florescence, magnetism, thermal response and/or zeta potential modulation.

14. The adjuvant as claimed in claim 1, wherein the non-stoichiometric adjuvant wherein the adjuvant is further doped with metallic and non-metallic impurities such as Fe, Mo, Cu, Na, K, Co, Si, Sn, Se, F, Cl, Br, I, Gd, Y, Tc, etc. to enhance its adjuvant activity and/or image contrast properties.

15. A composition comprising the nano- or micro-structures of the non-stoichiometric adjuvant as claimed in claim 1, and the pharmaceutically acceptable excipients and optionally comprises one or more active components are selected from:
a. immunogenic antigen;
b. antibodies; or its fragments; or
c. other immune-stimulating materials;
wherein the composition is a vaccine or a stand-alone immunomodulator.

16. The composition as claimed in claim 15, wherein the immunogenic antigen is selected from proteins, peptides, carbohydrates, nucleic acids such as RNA, mRNA, dsRNA, miRNA, DNA, oligonucleotides, RNA/DNA-mimics, lipids, lipo-proteins derived from virus, bacteria, fungi, selected from Tuberculosis, Anthrax, Tetanus, Leptospirosis, Pneumonia, Cholera, Botulism Pseudomonas Infection, MRSA Infection, E. Coli Infection, Meningitis, Gonorrhea, Bubonic Plague, Syphilis, Dengue, Zika, SARS-, MARS or tumor related antigens such as Tyrosinase-related protein (TRP)-2, Melanoma-associated Antigen-1 (MAGE-1), interleukin-13 (IL-13) receptor a2, Glycoprotein 100 (gp100), Antigen isolated from Immunoselected Melanoma-2 (AIM-2), Chondroitin sulfate proteoglycan 4 (CSPG4), High-molecular-weight melanoma-associated antigen (HMW-MAA), Melanoma proteoglycan (MPG), Melanoma antigen recognized by T-Cell-1 (MART-1), B melanoma antigen 1 (BAGE), Integrin α3β1, Transmembrane protein-tyrosine-phosphatases (PTP-LAR), CUB domain-containing protein 1 (CDCP1), The transferrin receptor 1 (TfR1), Prostatic acid phosphatase (PAP), Alpha-fetoprotein (AFP), Carcinoembryonic antigen (CEA), Mucin-1 (MUC-1), Epithelial tumor antigen (ETA), Melanoma associated antigen. (MAGE, Tumor protein p53, Annexin A2, Tropomodulin 3 (TMOD3), disintegrin and a metalloprotease 9 (ADAM9), Glypican-3 (GPC3), Hepatocellular carcinoma-associated antigen-587 (HCA587), Cancer/testis antigen 1B (CTAG1B), Human epidermal growth factor receptor 2 (HER-2/neu), Oncoprotein E6 and E7 of human papillomavirus 16 (HPV-16), Cellular retinoic acid-binding protein 1 (CRABP1), Folate receptor alpha (FOLR1), Kallikrein Related Peptidase (KLK10), Kallikrein-related peptidase 2 (KLK2/hK2), Human telomerase reverse transcriptase (hTERT), Cytotoxic T lymphocyte antigen 4 (CTLA-4), Prostate specific antigen (PSA), Prostate-Specific Membrane Antigen (PSMA), Hepatitis B surface antigen, Telomerase reverse transcriptase (TERT), Survivin, Lectin, galactoside binding Galectin-3 (LGALS3)Lectin, galactoside binding, soluble, Galectin-8 (LGALS8), v-Ki-ras2 Kirsten rat sarcoma virus oncogene homolog (KRAS), Embryonic stem cell expressed Ras (ERAS), Calcium voltage-gated channel auxiliary subunit gamma 1 (CACNG1), Alanyl aminopeptidase (ANPEP), F-Box Protein 6 (FBXO6), Endothelin Converting Enzyme 1 (ECE1), Tetraspanin-13 (TSPAN13), Neuron Cell Adhesion Molecule (N-CAM), Carcinoembryonic antigen (CEA), Epithelial cell adhesion molecule (Ep-CAM), Sialyl Tn sugar chains, Lewis antigens (Lewis-x, Lewis-b, Lewis-y structure), Globo H sugar chains, gangliosides such as GD2/GD3/GM2, Carbohydrate antigen 125, Carbohydrate antigen 199 Carbohydrate antigen 15-3, Tumor-associated glycoprotein 72 (TAG-72), Valosin-containing protein (p97), CD20, CD21, Mucin-16 (MUC-16), Epidermal growth factor receptor (EGFR), C-Met, receptor tyrosine kinase belonging to MET (MNNG HOS transforming gene) family, Tumor necrosis factor-related apoptosis-inducing ligand (TRAIL-R1), Insulin-Like Growth Factor 1 Receptor (IGF-1R), Vascular endothelial growth factor receptor (VEGF-R2/R1), Prostate stem cell antigen (PSCA), Growth/differentiation factor 8 (GDF8), Teratocarcinoma-derived growth factor 1 (TDGF1), Mucin-5AC gel-forming glycoprotein (MUC5AC), Carcinoembryonic antigen related cell adhesion molecules (CEACAM), Choline transporter-like protein 4 (SLC44A4), Neuropilin1, Glypican-3 (GPC3), EPH receptor A2 (EphA2), C-lectin, Pec60, Carbonic anhydrase IX (CAIX, MN, G250), Fibroblast activation protein α (FAPα), Matrix metalloproteinases (MMPs), Tumor endothelial markers (TEMs), among them TEM1 and TEM8, and/or TGF BETA receptor (CD105), dengue virus antigen, or zikka virus antigen

17. The composition as claimed in claim 15, wherein the other immune-stimulating materials mis selected from amorphous aluminum hydroxyphosphate sulfate (AAHS), Granulocyte-monocyte colony stimulating factor (GM-CSF), granulocyte-colony stimulating factor (G-CSF), monocyte-colony stimulating factor (m-CSF), saponins, imiquimod, Resiquimod (R-848), interleukins IL-1 to IL30; more specifically IL2, IL4, IL-5, IL12, IL13, IL20, IL15, IL21, IL1beta, interferon-alpha, -beta, -gamma, phosphonates, bisphosphonates, polyinosinic:polycytidylic acid, cell growth factors, immunogenic lipids/polymers/proteins, peptides, nucleic acids, Freund's Complete or incomplete adjuvant, lipo-polysaccharide, lecithin, Lyso-lecithin, lipids, cholesterol, Aluminum-based mineral salts (Alum), Calcium phosphate, AS04—contains 3-O-desacyl-4′-monophosphoryl lipid A (MPL), emulsions (e.g., MF59, Montanides), AS01B, CpG motifs (for eg; CpG 1018), Poly IC, AS03, ISCOMS, cholera toxin (CT), heat labile toxin (LT) from E. coli, virus-like particles, polymer adjuvant particles (e.g., PLG), virosomes, liposomal adjuvants, solid lipid adjuvants, carbohydrate adjuvants, synthetic DNA, viral like particles, RNA mimics, water in oil and oil in water containing olive, palm, coconut, peanut oil, squalene, mineral oils, aluminum monostearate; mineral gels such as aluminum hydroxide, alum, amorphous aluminium phosphate sulfate, aluminium adjuvants, calcium phosphate, aluminum phosphate hexadecylamine, octadecylamine, dimethyldioctadecyl-ammonium bromide, N,N-dioctadecyl-N′,N′-bis(2-hydroxymethyl) propanediamine, methoxyhexadecylglycerol and pluronic polyols; polyanions such as pyran, protamine sulfate, polyethyleneimne, dextran sulfate, polyacrylic acid and carbopol or combinations thereof.

18. Use of the non-stoichiometric adjuvant as claimed in claim 1, wherein the adjuvant compound in combination with other therapy of cancer or infectious or autoimmune diseases such as chemotherapeutics, small molecule inhibitors, tumor micro-environment targeted therapies, anti-macrophage therapy, anti-myeloid derived immunosuppressor cell therapy, anti-T reg therapy, immune check point blockade therapy, immunotherapy, antibody therapy, cytokine therapy, radiation therapy, nuclear medicine, photodynamic therapy, tumour killing field therapy, surgery, anti-viral therapy, ayurvedic, homeopathic and/or traditional medicine therapies.

19. Use of the non-stoichiometric adjuvant compound as claimed in claim 1 alone or in combination with one or more cell therapies or antibody therapies wherein the cell therapy is selected from CART-T cell, CAR-Macrophage, DC cell vaccine, NK cell therapy, adoptive T cell therapies, and/or stem-cell therapy and wherein the antibody therapy is selected from immune-checkpoint blockade antibodies against PD1, PDL1, CTLA4, Lag3, IDO, TIM3, growth factor binding antibodies against EGFR, PDGFR, VEGF, VEGFR, Her2/nue, IL6, TNF alpha inhibitor antibody.

20. A method of treating a subject by generating an immune response in subject injected with the composition as claimed in claim 15, wherein the immune response is generated by way of activating innate and adaptive immune cells such as DC, macrophages, B cells and T cells and producing specific and non-specific antibodies and T, NK cell, Dendritic cell, macrophage, neutrophil response against the disease condition or antigen used in the vaccine formulation.

21. The method as claimed in claim 20, wherein the cancer is lung, kidney, breast, pancreas, brain, colon, intestine, skin, liver, neuroendocrine organs, oesophagus, stomach, blood cells, muscle cells, bone, head & neck, adenocarcinoma, squamous cell carcinoma, stromal cell carcinoma, soft-tissue carcinoma, sarcomas, lymphoma, myeloma, by way of nanoparticle alone by activating immune cells, or in combination with other immunotherapeutics such as anti-MDSC therapeutics, 5fluorourasil, capecitabin, Gemcitabine, or combinations thereof.

22. The method as claimed in claim 20, wherein the adjuvant is used in combination with an anti-MDSC depletion therapy molecule 5FU or its prodrug; capecitabine for repolarizing M2 macrophages to M1, enhancing cytotoxic T cell infiltration into the tumor and development of memory T cells into lung tumor

23. The method as claimed in claim 20, wherein the adjuvant is used as vaccine composition by combining it with mRNA or peptide antigens of Dengue virus or zikka virus.
